(19) United States
(12) Patent Application Publication
(10) Pub. No.: US 2025/0127719 A1
(43) Pub. Date: Apr. 24, 2025

(54) ANISAMIDE-CONTAINING LIPIDS AND COMPOSITIONS AND METHODS OF USE THEREOF

(71) Applicant: The Trustees Of The University Of Pennsylvania, Philadelphia, PA (US)

(72) Inventors: Michael J. Mitchell, Philadelphia, PA (US); Xuexiang Han, Philadelphia, PA (US)

(73) Assignee: The Trustees Of The University Of Pennsylvania, Philadelphia, PA (US)

(21) Appl. No.: 18/716,088

(22) PCT Filed: Dec. 6, 2022
(86) PCT No.: PCT/US22/80983

§ 371 Date: Jun. 3, 2024

Related U.S. Application Data
(60) Provisional application No. 63/286,760, filed on Dec. 7, 2021.

(51) Int. Cl. A61K 9/127; A61K 48/00; C07C 235/50; C07D 295/13; C12N 15/113

(52) U.S. Cl. CPC A61K 9/127; A61K 48/0033; C07C 235/50; C07D 295/13; C12N 15/113; C12N 2310/11; C12N 2310/113; C12N 2310/14; C12N 2310/141

(57) ABSTRACT

The present disclosure relates to ionizable lipidoid compounds comprising an anisamide moiety, and lipid nanoparticles (LNPs) comprising the same. In certain embodiments, the LNP selectively binds to at least one sigma receptor. In certain embodiments, the LNP specifically targets a cell of interest (e.g., a cell expressing a sigma receptor, fibroblast, cancer cell, stromal cell, and epithelial cell, inter alia). In another aspect, the present disclosure provides methods for in vivo delivery of therapeutic agents to treat, prevent, and/or ameliorate diseases and/or disorders, including but not limited to fibrosis and cancer.

### CROSS-REFERENCE TO RELATED APPLICATIONS

This application claims priority under 35 U.S.C. § 119(e) to U.S. Provisional Patent Application No. 63/286,760, filed Dec. 7, 2021, which application is incorporated herein by reference in its entirety.

### STATEMENT REGARDING FEDERALLY SPONSORED RESEARCH OR DEVELOPMENT

This invention was made with government support under TR002776 awarded by the National Institutes of Health. The government has certain rights in the invention.

### BACKGROUND

With the development of RNA therapeutics, gene therapy, and gene editing technologies, inter alia, it is necessary to address the challenge of RNA delivery to cells in a precise and efficient way. Currently, there are three FDA approved/EUA products that utilize lipid nanoparticles (LNPs), including Onpattro (siRNA) and the Pfizer and Moderna mRNA COVID-19 vaccines. For example, ND-L02-s0201/BMS-986263 is a LNP drug product containing a heat shock protein 47 (HSP47)-specific siRNA and is being developed for the treatment of liver and idiopathic pulmonary fibrosis. However, the lipid use in this LNP is a cationic lipid, which are more toxic than the ionizable lipids.

Furthermore, another challenge of LNP development is to achieve targeted delivery to a range of cells in the body.

Thus, there is a need in the art for a platform for the delivery of RNA therapeutics, gene therapies, and gene editing technologies, inter alia, in a precise and efficient way to a cell of interest. The present disclosure addresses this unmet need.

### BRIEF SUMMARY OF THE INVENTION

In one aspect, the present disclosure provides a compound of Formula (I), or a salt, solvate, stereoisomer, tautomer, or isotopologue thereof:

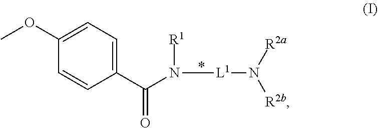

wherein:
L¹ is selected from the group consisting of -(optionally substituted C₂-C₁₂ heteroalkylenyl)- and -(optionally substituted C₂-C₁₂ heteroalkylenyl)-(optionally substituted C₂-C₁₂ heterocycloalkylenyl)-,
   wherein each occurrence of C₂-C₁₂ heteroalkylenyl and C₂-C₁₂ heterocycloalkylenyl is optionally substituted with at least one substituent selected from the group consisting of optionally substituted C₁-C₂₄ alkyl, optionally substituted C₁-C₂₄ heteroalkyl optionally substituted C₃-C₈ cycloalkyl, optionally substituted C₆-C₁₀ aryl, optionally substituted C₂-C₁₀ heteroaryl, R^{2c}, and

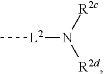

or two geminal substituents may combine to form ═O;
each occurrence of L² is independently selected from the group consisting of -(optionally substituted C₁-C₆ alkylenyl)-, -(optionally substituted C₂-C₆ heteroalkylenyl)-, -(optionally substituted C₁-C₆ alkylenyl)-C(═O)X-(optionally substituted C₁-C₆ alkylenyl)-, and -(optionally substituted C₁-C₆ alkylenyl)-XC(═O)-(optionally substituted C₁-C₆ alkylenyl)-;
R¹ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{2a}, R^{2b}, R^{2c}, and R^{2d}, if present, is independently selected from the group consisting of H, -(optionally substituted C₁-C₆ alkylenyl)-C(═O)OR³, -(optionally substituted C₁-C₆ alkylenyl)-C(═O)N(R³)(R⁴), -(optionally substituted C₁-C₆ alkylenyl)-C(═O)R³, -(optionally substituted C₁-C₆ alkylenyl)-(R³), —C(═O)OR³, —C(═O)N(R³)(R⁴), —C(═O)R³, and R³,
   wherein no more than one of each occurrence of R^{2a}, R^{2b}, R^{2c}, and R^{2d} is H;
R³ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁴ is selected from the group consisting of H and optionally substituted C₁-C₆ alkyl; each occurrence of X is independently selected from the group consisting of a bond, O, and NR⁵; and
each occurrence of R⁵ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

In certain embodiments, the compound of Formula (I) is selected from the group consisting of:

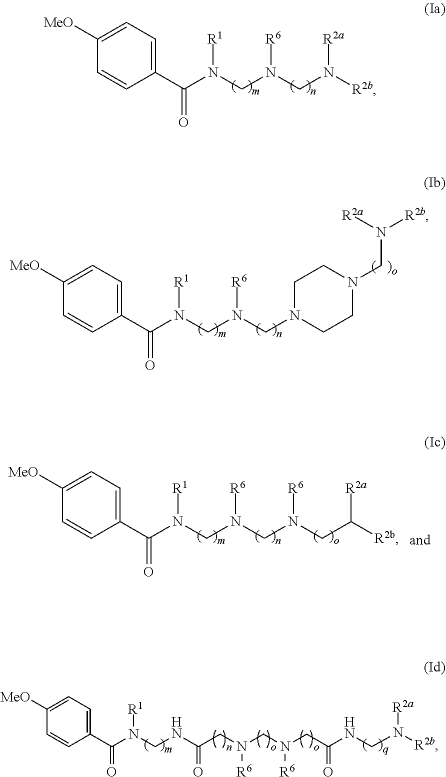

wherein:
each occurrence of R⁶ is independently selected from the group consisting of H, optionally substituted C₁-C₂₄ alkyl, and

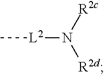

m, n, o, p, and q are each independently an integer selected from the group consisting of 1, 2, 3, and 4.

In certain embodiments, the compound of Formula (I) is selected from the group consisting of:

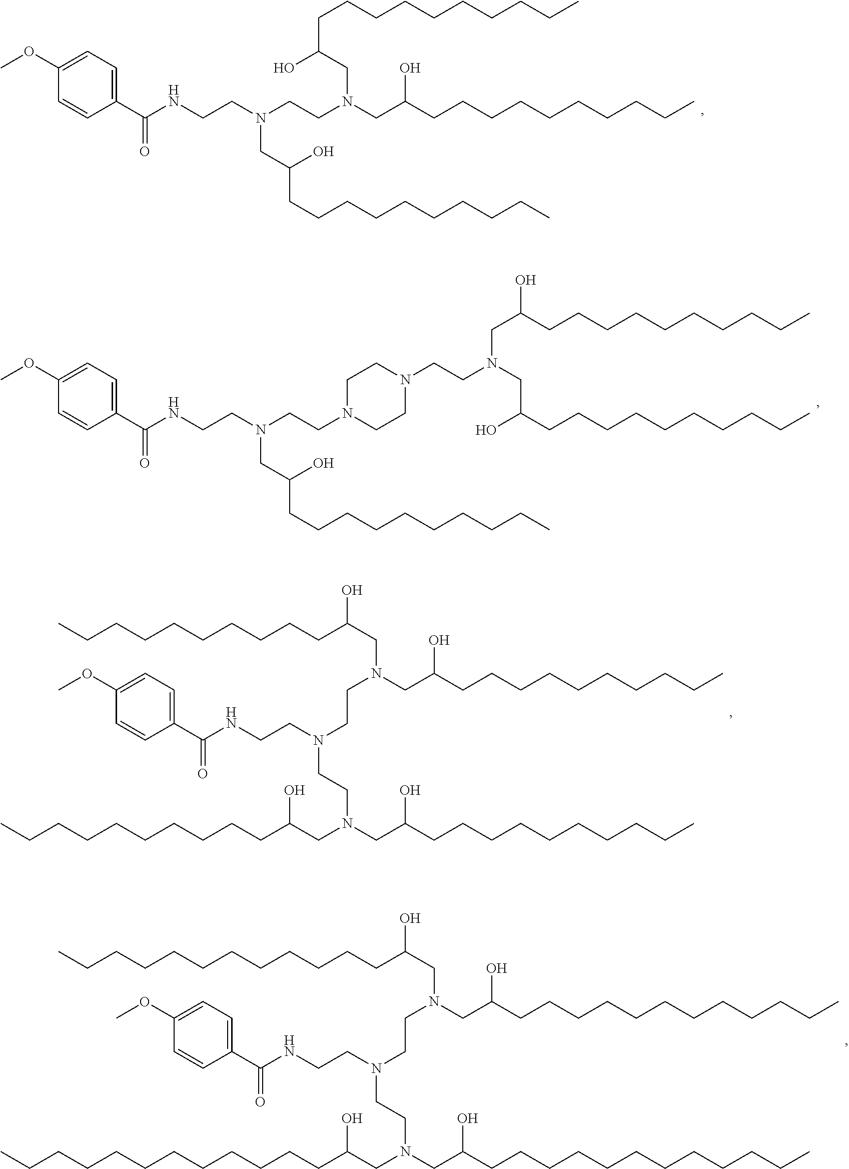

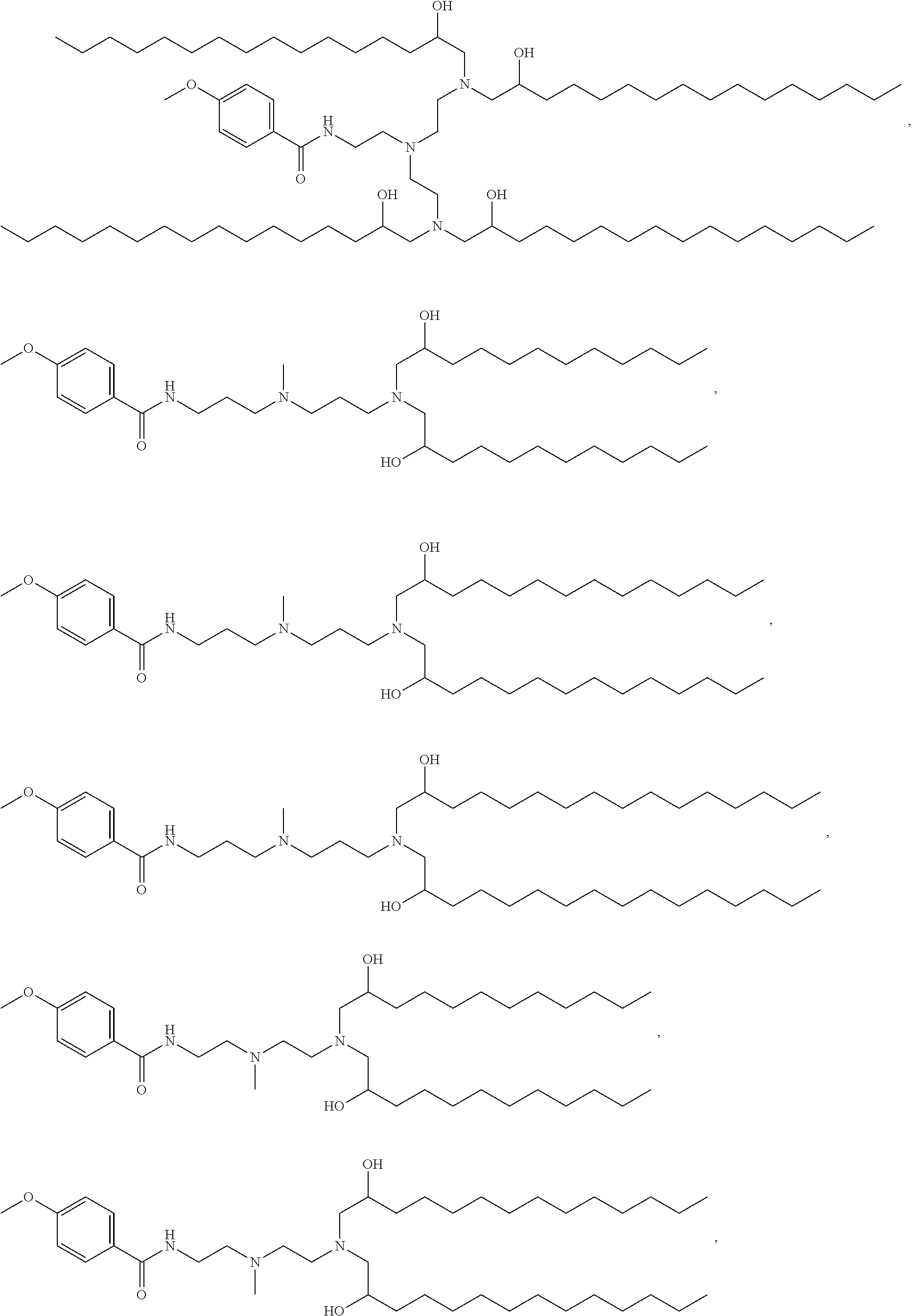

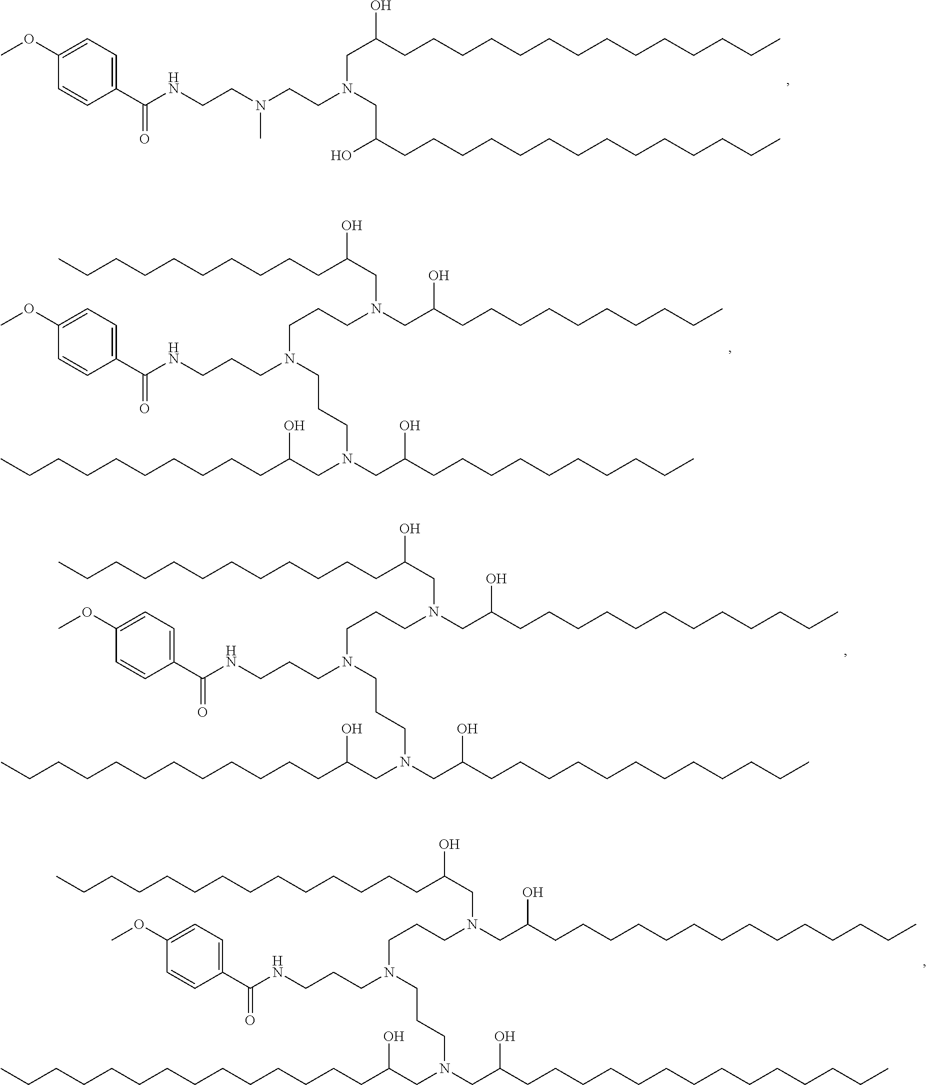

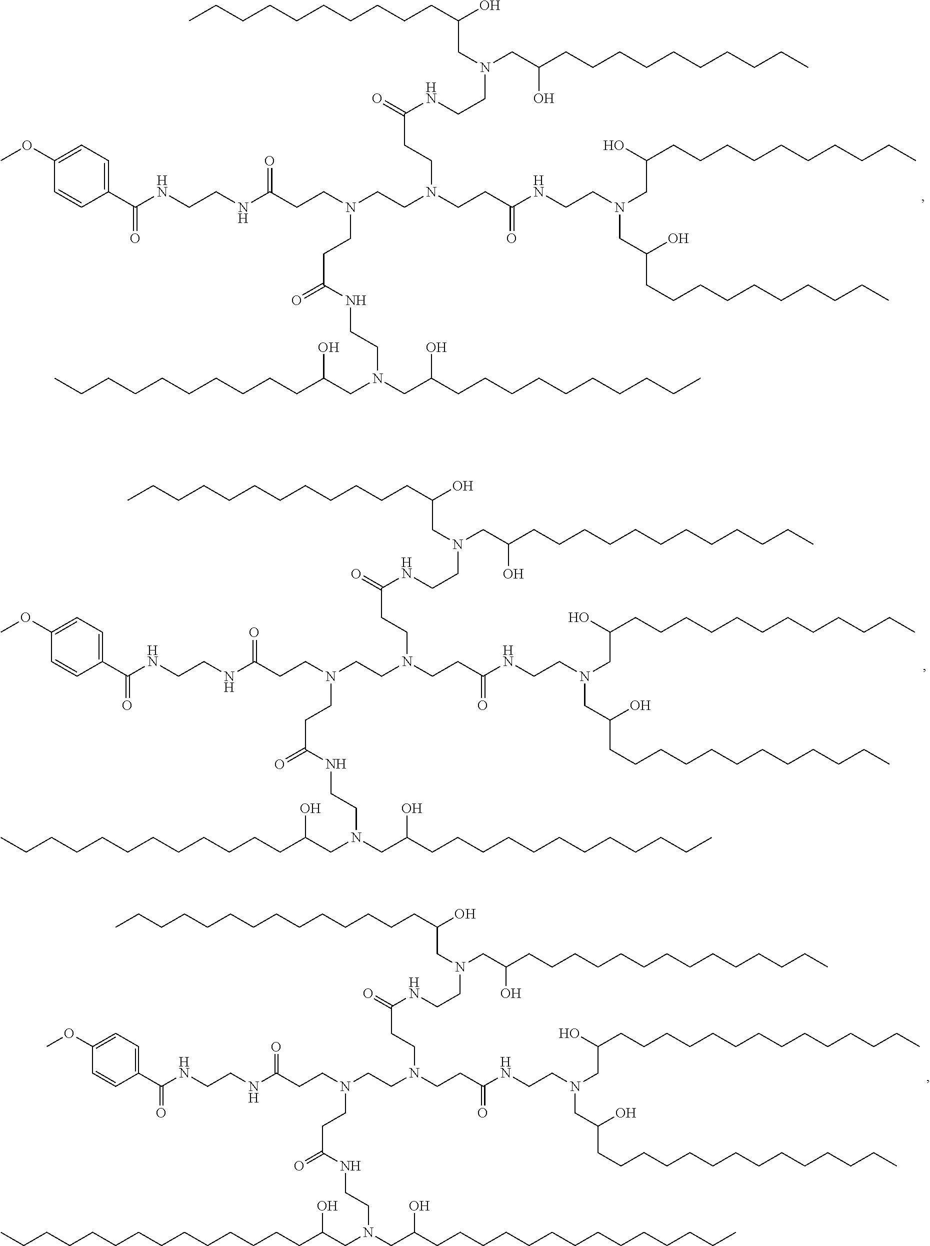

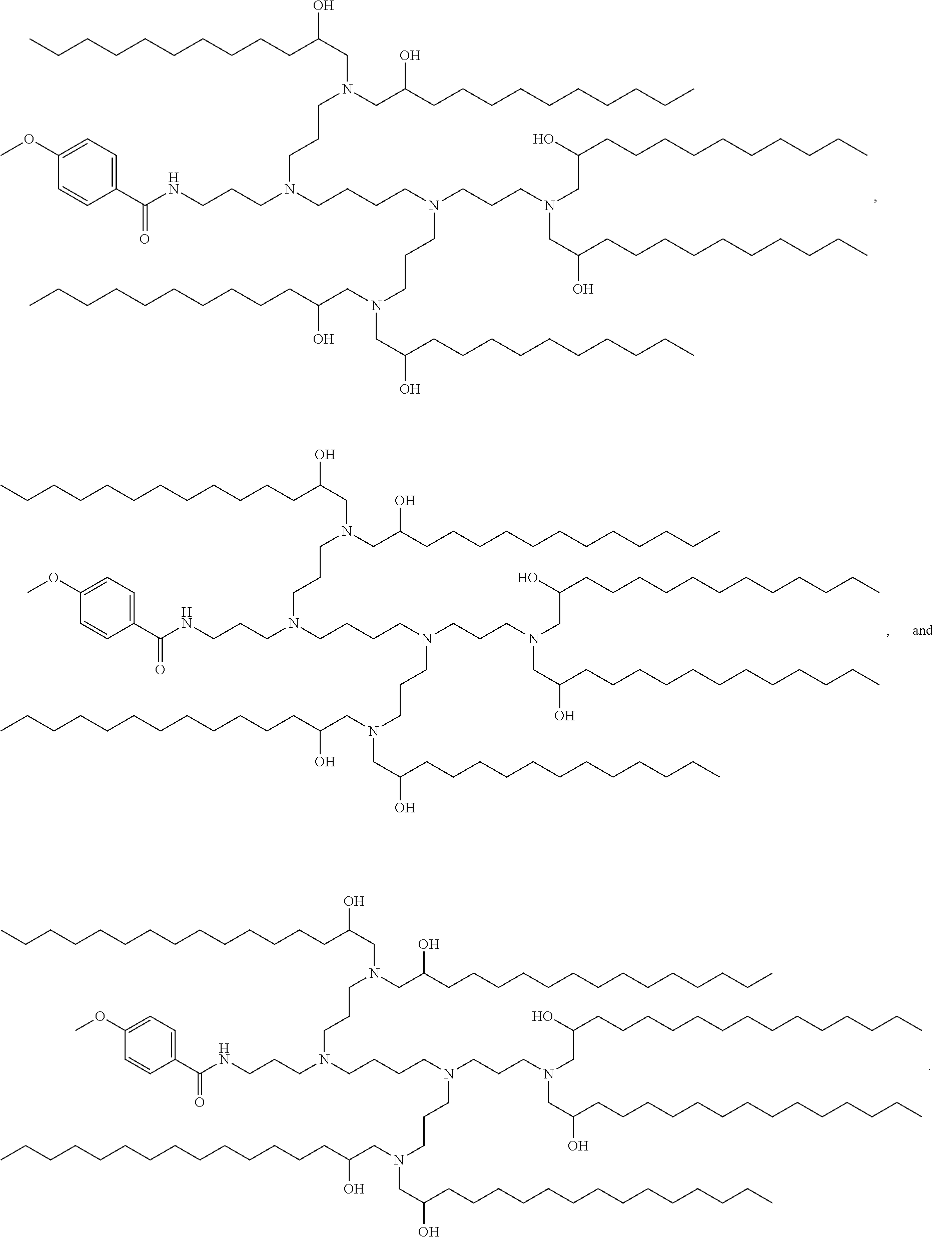

In another aspect, the present disclosure provides a lipid nanoparticle (LNP). In certain embodiments, the LNP comprises at least one compound of the present disclosure (i.e., compound of Formula (I)). In certain embodiments, the LNP comprises at least one helper lipid. In certain embodiments, the LNP comprises at least one cholesterol lipid. In certain embodiments, the LNP comprises at least one conjugated lipid.

In certain embodiments, the compound is

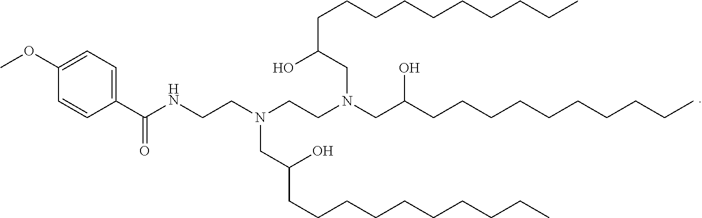 [SMILES: CCCCCCCCCCC(O)CN(CCNC(=O)c1ccc(OC)cc1)CCN(CC(O)CCCCCCCCCC)CC(O)CCCCCCCCCC]

In certain embodiments, the compound is

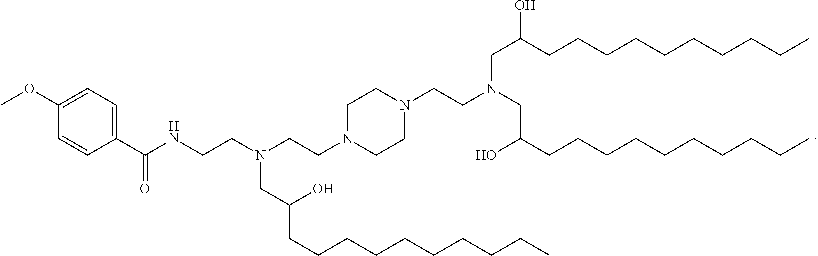 [SMILES: CCCCCCCCCCC(O)CN(CCNC(=O)c1ccc(OC)cc1)CCN1CCN(CCN(CC(O)CCCCCCCCCC)CC(O)CCCCCCCCCC)CC1]

In certain embodiments, the compound is

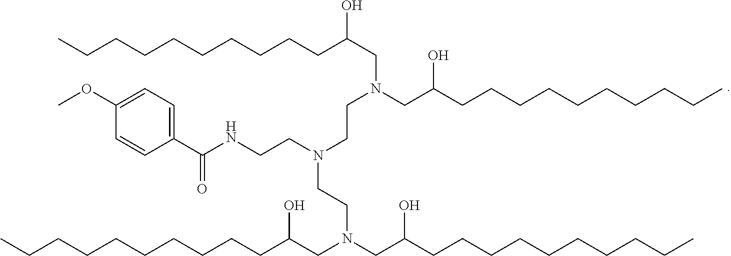 [SMILES: CCCCCCCCCCC(O)CN(CCN(CCNC(=O)c1ccc(OC)cc1)CCN(CC(O)CCCCCCCCCC)CC(O)CCCCCCCCCC)CC(O)CCCCCCCCCC]

In certain embodiments, the compound is

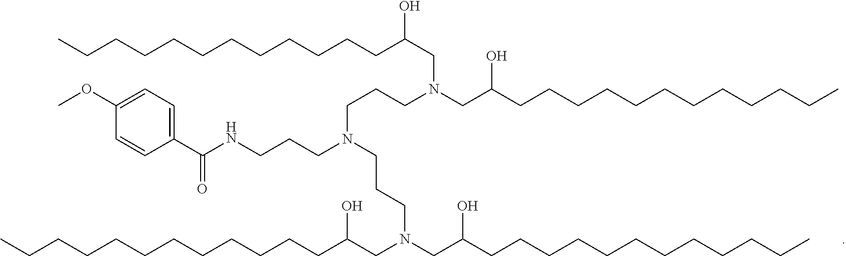 [SMILES: CCCCCCCCCCCCC(O)CN(CCCN(CCCNC(=O)c1ccc(OC)cc1)CCCN(CC(O)CCCCCCCCCCCC)CC(O)CCCCCCCCCCCC)CC(O)CCCCCCCCCCCC]

In certain embodiments, the compound is

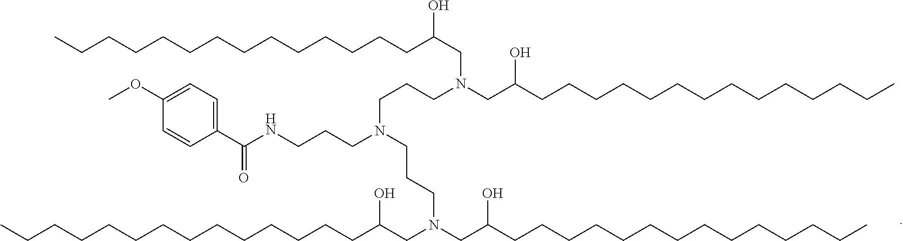 [SMILES: CCCCCCCCCCCCCCC(O)CN(CCCN(CCCNC(=O)c1ccc(OC)cc1)CCCN(CC(O)CCCCCCCCCCCCCC)CC(O)CCCCCCCCCCCCCC)CC(O)CCCCCCCCCCCCCC]

In certain embodiments the compound is

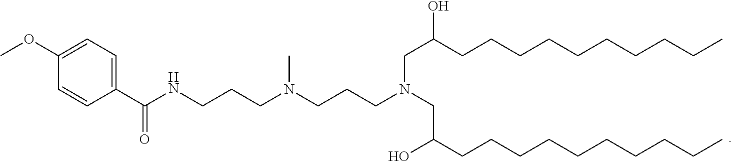 [SMILES: CCCCCCCCCCC(O)CN(CCCN(C)CCCNC(=O)c1ccc(OC)cc1)CC(O)CCCCCCCCCC]

In certain embodiments, the compound is

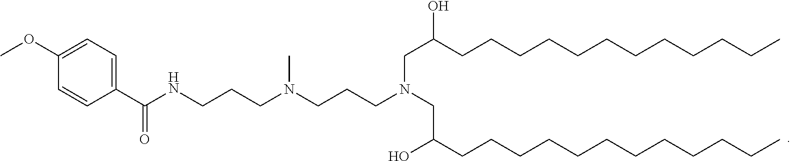 [SMILES: CCCCCCCCCCCCC(O)CN(CCCN(C)CCCNC(=O)c1ccc(OC)cc1)CC(O)CCCCCCCCCCCC]

In certain embodiments the compound is

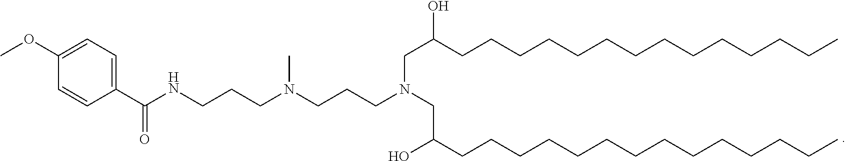 [SMILES: CCCCCCCCCCCCCCC(O)CN(CCCN(C)CCCNC(=O)c1ccc(OC)cc1)CC(O)CCCCCCCCCCCCCC]

In certain embodiments, the compound is

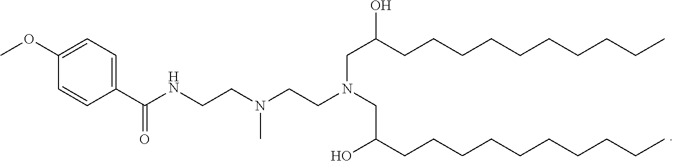 [SMILES: CCCCCCCCCCC(O)CN(CCN(C)CCNC(=O)c1ccc(OC)cc1)CC(O)CCCCCCCCCC]

In certain embodiments the compound is

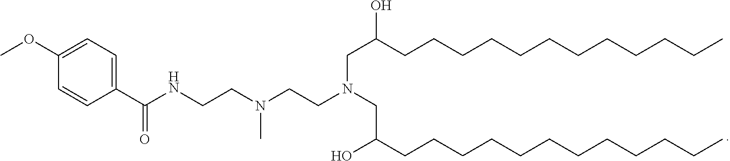 [SMILES: CCCCCCCCCCCCC(O)CN(CCN(C)CCNC(=O)c1ccc(OC)cc1)CC(O)CCCCCCCCCCCC]

In certain embodiments, the compound is

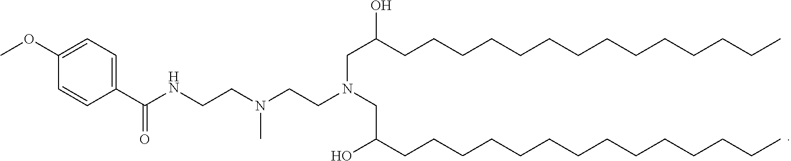 [SMILES: CCCCCCCCCCCCCCC(O)CN(CCN(C)CCNC(=O)c1ccc(OC)cc1)CC(O)CCCCCCCCCCCCCC]

In certain embodiments, the compound is

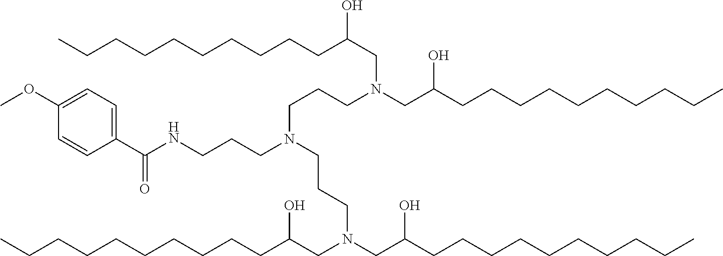 [SMILES: CCCCCCCCCCC(O)CN(CCCN(CCCNC(=O)c1ccc(OC)cc1)CCCN(CC(O)CCCCCCCCCC)CC(O)CCCCCCCCCC)CC(O)CCCCCCCCCC]

In certain embodiments the compound is

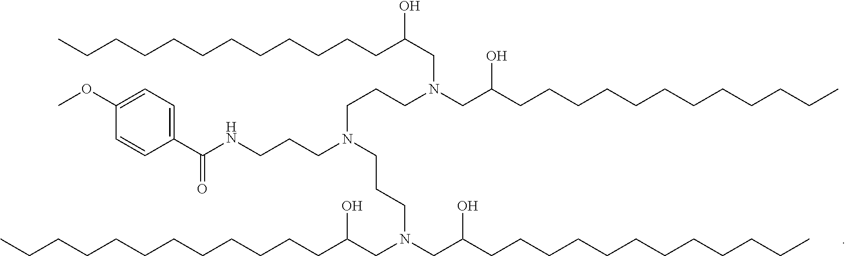 [SMILES: CCCCCCCCCCCCC(O)CN(CCCN(CCCNC(=O)c1ccc(OC)cc1)CCCN(CC(O)CCCCCCCCCCCC)CC(O)CCCCCCCCCCCC)CC(O)CCCCCCCCCCCC]

In certain embodiments, the compound is

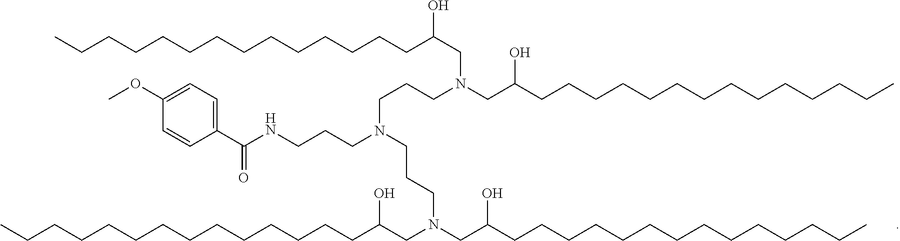 [SMILES: CCCCCCCCCCCCCCC(O)CN(CCCN(CCCNC(=O)c1ccc(OC)cc1)CCCN(CC(O)CCCCCCCCCCCCCC)CC(O)CCCCCCCCCCCCCC)CC(O)CCCCCCCCCCCCCC]

In certain embodiments, the compound is

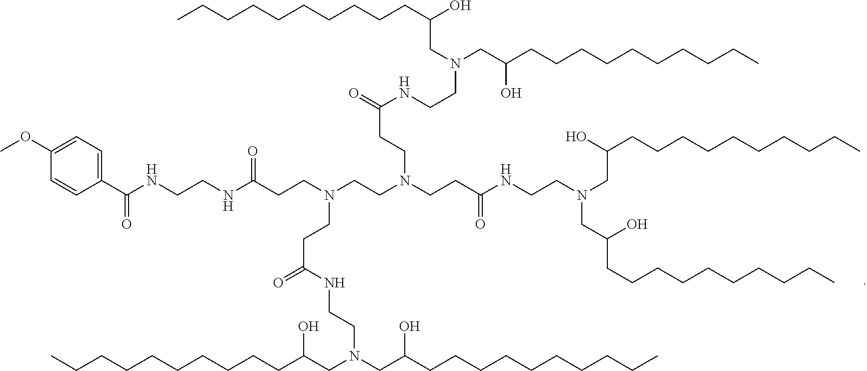 [SMILES: CCCCCCCCCCC(O)CN(CCNC(=O)CCN(CCC(=O)NCCNC(=O)c1ccc(OC)cc1)CCN(CCC(=O)NCCN(CC(O)CCCCCCCCCC)CC(O)CCCCCCCCCC)CCC(=O)NCCN(CC(O)CCCCCCCCCC)CC(O)CCCCCCCCCC)CC(O)CCCCCCCCCC]

In certain embodiments, the compound is

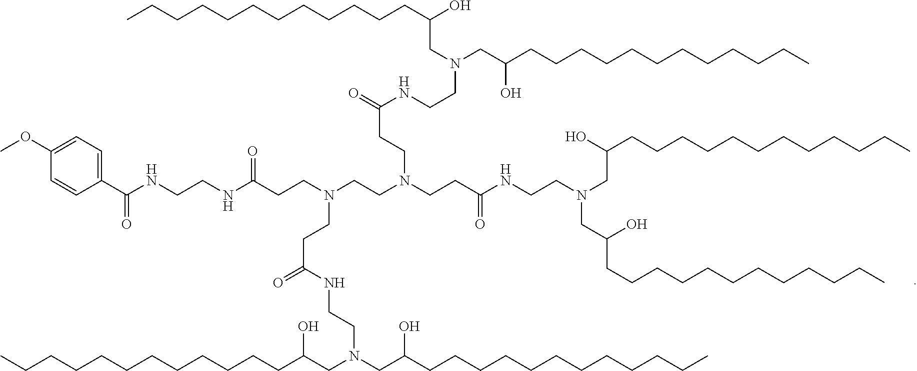 [SMILES: CCCCCCCCCCCCC(O)CN(CCNC(=O)CCN(CCC(=O)NCCNC(=O)c1ccc(OC)cc1)CCN(CCC(=O)NCCN(CC(O)CCCCCCCCCCCC)CC(O)CCCCCCCCCCCC)CCC(=O)NCCN(CC(O)CCCCCCCCCCCC)CC(O)CCCCCCCCCCCC)CC(O)CCCCCCCCCCCC]

In certain embodiments, the compound is

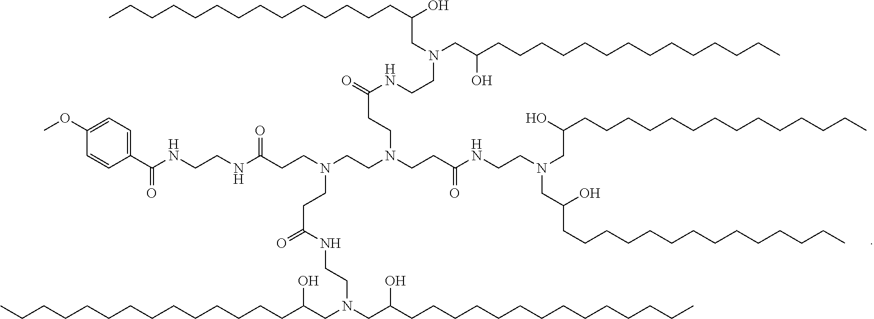 [SMILES: CCCCCCCCCCCCCCC(O)CN(CCNC(=O)CCN(CCC(=O)NCCNC(=O)c1ccc(OC)cc1)CCN(CCC(=O)NCCN(CC(O)CCCCCCCCCCCCCC)CC(O)CCCCCCCCCCCCCC)CCC(=O)NCCN(CC(O)CCCCCCCCCCCCCC)CC(O)CCCCCCCCCCCCCC)CC(O)CCCCCCCCCCCCCC]

In certain embodiments, the compound is

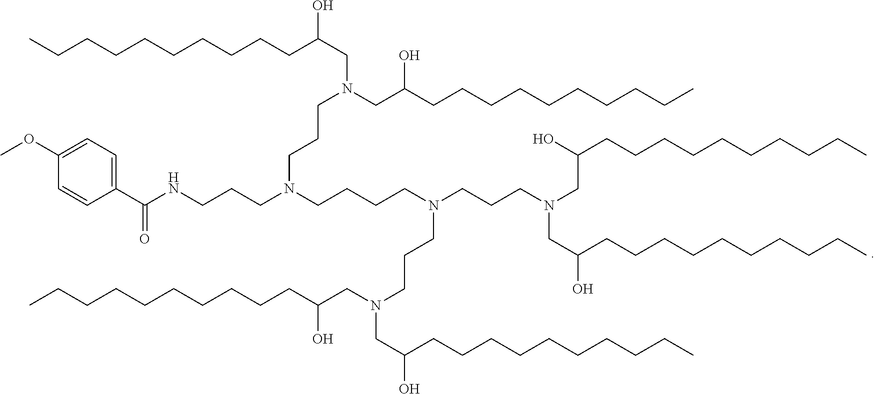 [SMILES: CCCCCCCCCCC(O)CN(CCCN(CCCCN(CCCN(CC(O)CCCCCCCCCC)CC(O)CCCCCCCCCC)CCCN(CC(O)CCCCCCCCCC)CC(O)CCCCCCCCCC)CCCNC(=O)c1ccc(OC)cc1)CC(O)CCCCCCCCCC]

In certain embodiments, the compound is

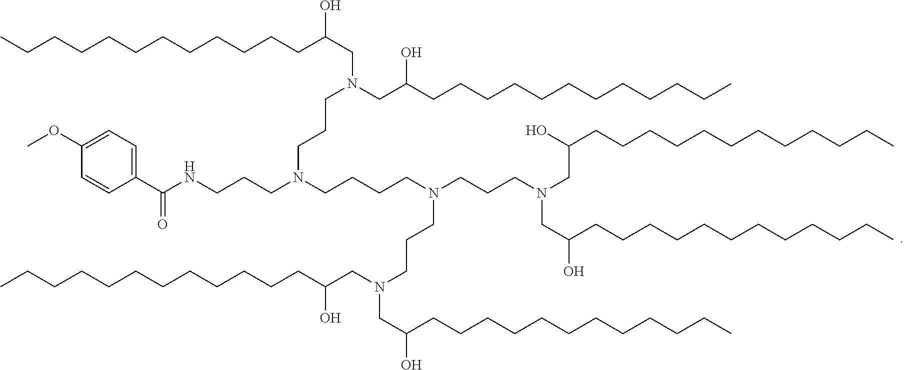 [SMILES: CCCCCCCCCCCCC(O)CN(CCCN(CCCCN(CCCN(CC(O)CCCCCCCCCCCC)CC(O)CCCCCCCCCCCC)CCCN(CC(O)CCCCCCCCCCCC)CC(O)CCCCCCCCCCCC)CCCNC(=O)c1ccc(OC)cc1)CC(O)CCCCCCCCCCCC]

In certain embodiments, the compound is

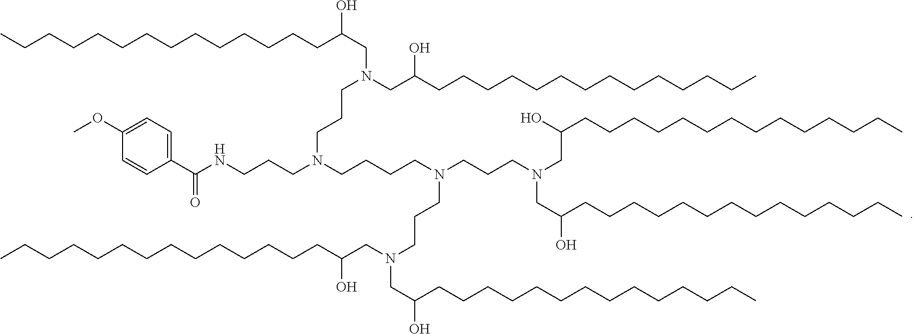 [SMILES: CCCCCCCCCCCCCCC(O)CN(CCCN(CCCCN(CCCN(CC(O)CCCCCCCCCCCCCC)CC(O)CCCCCCCCCCCCCC)CCCN(CC(O)CCCCCCCCCCCCCC)CC(O)CCCCCCCCCCCCCC)CCCNC(=O)c1ccc(OC)cc1)CC(O)CCCCCCCCCCCCCC]

In certain embodiments, the ionizable lipid compound of Formula (I) is:

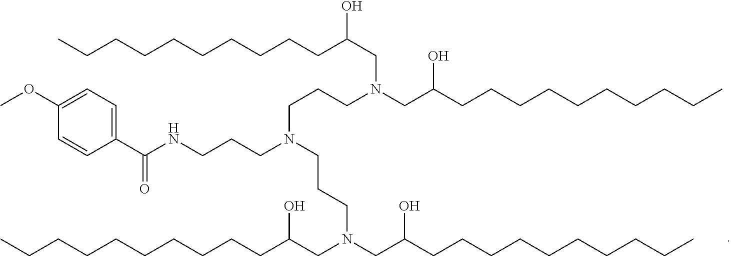 [SMILES: CCCCCCCCCCC(O)CN(CCCN(CCCNC(=O)c1ccc(OC)cc1)CCCN(CC(O)CCCCCCCCCC)CC(O)CCCCCCCCCC)CC(O)CCCCCCCCCC]

In certain embodiments, the compound comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the compound comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the compound comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the compound comprises about 50 mol % of the LNP. In certain embodiments, the compound comprises less than about 50 mol % of the LNP. In certain embodiments, the compound comprises more than about 50 mol % of the LNP.

In certain embodiments, the at least one helper lipid comprises distearoylphosphatidylcholine (DSPC).

In certain embodiments, the at least one helper lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one helper lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one helper lipid comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one helper lipid comprises about 10 mol % of the LNP. In certain embodiments, the at least one helper lipid comprises less than about 10 mol % of the LNP. In certain embodiments, the at least one helper lipid comprises more than about 10 mol % of the LNP.

In certain embodiments, the at least one cholesterol lipid comprises cholesterol.

In certain embodiments, the at least one cholesterol lipid comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the at least one cholesterol lipid comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the at least one cholesterol lipid comprises more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the cholesterol lipid comprises about 38.5 mol % of the LNP. In certain embodiments, the cholesterol lipid comprises less than about 38.5 mol % of the LNP. In certain embodiments, the cholesterol lipid comprises more than about 38.5 mol % of the LNP.

In certain embodiments, the at least one conjugated lipid comprises C14-PEG2000.

In certain embodiments, the at least one conjugated lipid comprises about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, or about 12.5 mol % of the LNP.

In certain embodiments, the at least one conjugated lipid comprises less than about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, or about 12.5 mol % of the LNP.

In certain embodiments, the at least one conjugated lipid comprises more than about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, or about 12.5 mol % of the LNP.

In certain embodiments, the ratio of (a):(b):(c):(d) is about 50:10:38.5:1.5.

In another aspect, the present disclosure provides a pharmaceutical composition comprising the LNP of the present disclosure and at least one pharmaceutically acceptable carrier.

In another aspect, the present disclosure provides a method of delivering an agent to a subject in need thereof, the method comprising administering to the subject a therapeutically effective amount of at least one LNP of the present disclosure, wherein the agent is at least partially encapsulated in the LNP.

In another aspect, the present disclosure provides a method of treating, preventing, and/or ameliorating a disease or disorder in a subject, the method comprising administering to the subject the LNP of the present disclosure and/or the pharmaceutical composition of the present disclosure.

In certain embodiments, the disease or disorder is fibrosis.

In certain embodiments, the disease or disorder is selected from the group consisting of a fibrotic disease or disorder, a disease or disorder associated with fibrosis, a neurological disease or disorder, a skin condition, a disease or disorder associated with the level of activity of at least one sigma receptor, a cancer or a disease or disorder associated therewith, and any combination thereof.

In certain embodiments, the fibrotic disease or disorder is at least one selected from the group consisting of pulmonary fibrosis, cystic fibrosis, fibrothorax, idiopathic pulmonary fibrosis, bridging fibrosis, cirrhosis, glial scar, liver fibrosis, myocardial fibrosis, interstitial fibrosis, replacement fibrosis, arterial stiffness, arthrofibrosis, chronic kidney disease, Crohn's disease, dupuytren's contracture, keloid, mediastinal fibrosis, myelofibrosis, peyronie's disease, nephrogenic systemic fibrosis, progressive massive fibrosis, retroperitoneal fibrosis, scleroderma, systemic sclerosis, adhesive capsulitis, and any combination thereof.

In another aspect, the present disclosure provides a modified cell produced by administering at least one LNP of the present disclosure or a composition comprising the same to a cell. In certain embodiments, the cell expresses a chimeric antigen receptor (CAR).

### BRIEF DESCRIPTION OF THE FIGURES

The drawings illustrate generally, by way of example, but not by way of limitation, various embodiments of the present application.

FIGS. 1A-1E depict preparation and application of ligand-tethered lipidoid nanoparticles for targeted siRNA delivery to HSCs to treat liver fibrosis. FIG. 1A: formulation of AA-T3A-C12/siHSP47 LNP via microfluidic mixing. The ethanol lipid solution containing anisamide-tethered lipidoid (AA-T3A-C12), phospholipid (DSPC), PEG-lipid (C14-PEG) and cholesterol is rapidly mixed with an acidic aqueous solution containing HSP47 siRNA in a microfluidic device to formulate AA-T3A-C12/siHSP47 LNP. FIG. 1B: scheme of targeted AA-T3A-C12/siHSP47 LNP delivery to activated HSCs to knockdown HSP47 and treat liver fibrosis. HSCs are located in the space of Disse, an area between LSECs and hepatocytes. After rapidly shedding PEG in circulation, the LNP will expose multivalent anisamide ligands on its surface that can strongly bind with sigma receptors overexpressed on activated HSCs to mediate cellular uptake. FIG. 1C depicts a schematic representation of fibrosis. FIG. 1D depicts schematic representations of fibroblast-targeted LNP platforms and the role of heat shock protein (HSP47) in collagen processing. FIG. 1E depicts representative results demonstrating that sigma receptors are overexpressed in activated fibroblasts.

FIG. 2 depicts an exemplary mass spectrum of anisoyl-N-hydroxysuccinimide (NHS).

FIG. 3 depicts an exemplary ¹H-NMR spectrum of anisoyl-NHS in DMSO-d₆.

FIG. 4 depicts an exemplary mass spectrum of lipidoid AA-T3A-C12.

FIG. 5 depicts an exemplary ¹H-NMR spectrum of lipidoid AA-T3A-C12 in DMSO-d₆. The ¹H-NMR spectrum of AA-T3A-C12 showed characteristic peaks of anisamide, T3A core, and epoxide alkyl tail.

FIGS. 6A-6C depict uncropped Western blots of FIG. 19D (FIG. 6A), FIG. 19E (FIG. 6B), and FIG. 24F (FIG. 6C).

FIGS. 7A-7E depict synthesis and screening of AA-lipidoids for targeted RNA delivery to activated fibroblasts. FIG. 7A: one-pot, two-step modular synthesis of AA-lipidoids. A representative synthesis of AA-T3A-C12 is shown. Anisoyl-NHS, polyamines and epoxides were used to build a combinatorial library of 18 AA-lipidoids. FIG. 7B: first round screening of lipidoids and AA-lipidoids with high potency. Lipidoids without anisamide were synthesized by traditional ring-opening reaction between epoxides and polyamines. GFP siRNA-loaded LNPs were formulated to treat activated 3T3-GFP fibroblasts for 48 h to obtain their knockdown efficiency. The dash line indicates 80% GFP knockdown. FIG. 7C: statistical analysis of structure-activity relationship. GFP knockdown efficiency was plotted based on lipidoids with or without anisamide. FIG. 7D: second round screening of lipidoids and AA-lipidoids with high dependency on sigma receptor-mediated transfection. Activated 3T3-GFP fibroblasts were pre-treated with haloperidol (HP) to block sigma receptor before GFP siRNA-loaded LNPs treatment. FIG. 7E: statistical analysis of the relationship between sigma receptor blocking and knockdown efficiency. GFP knockdown efficiency was plotted based on treatment with or without HP. Data are presented as mean±SD (n=3). ns, not significant; **p<0.01.

FIGS. 8A-8C depict a pilot in vitro screening of three representative polyamine cores with C12 epoxide tail. FIG. 8A: structures of piperazine derivative 200, linear amine 114 and branched amine 110. FIG. 8B: GFP knockdown at 48 h post-treatment with 50 nM siGFP. FIG. 8C: cell viability at 48 h post-treatment. Data are presented as mean±SD (n=3).

FIG. 9 provides a bar graph depicting cell viability at 48 h post-treatment. Cell viabilities were above 80% for all LNPs. Data are presented as mean±SD (n=3).

FIG. 10 depicts MC3 LNP-mediated GFP knockdown. Activated 3T3-GFP fibroblasts were pre-treated with or without 30 μM haloperidol (HP) for 2 h before GFP siRNA-loaded MC3 LNP (50 nM) was used to treat cells for another 48 h. No obvious loss of silencing activity was observed after HP treatment. Data are presented as mean±SD (n=3). ns, not significant.

FIGS. 11A-11B depict optimization of the weight ratio of AA-T3A-C12 to siRNA. FIG. 11A: gel retardation assay. LNPs with different weight ratios of AA-T3A-C12:siRNA were analyzed by 1% agarose gel electrophoresis. 500 ng siRNA was loaded into each lane. Little to no free siRNA was observed when the weight ratio of AA-T3A-C12:siRNA was above 5:1. FIG. 11B: RiboGreen RNA assay. The siRNA encapsulation efficiency reached a plateau of ˜90% when the weight ratio of AA-T3A-C12:siRNA was 10:1. Data are presented as mean±SD (n=3).

FIGS. 12A-12D depict characterization of AA-T3A-C12 LNP and cellular uptake. FIG. 12A: representative TEM image of AA-T3A-C12/siRNA LNP. Scale bar, 100 nm. FIG. 12B: flow cytometry analysis of cellular uptake of Cy5-siRNA-loaded LNPs with or without haloperidol (HP) pretreatment. FIGS. 12C-12D: flow cytometry analysis of competitive cellular uptake of Cy5-siRNA-loaded LNPs in a fibroblast/hepatocyte (3T3-GFP/H2.35) co-culture environment. The mean fluorescence intensity ratio between fibroblast and hepatocyte (MFI-r-GFP/MFI_{H2.35}) was calculated to indicate preferential uptake by fibroblasts over hepatocytes. Data are presented as mean±SD (n=3). **p<0.01; ***p<0.001.

FIG. 13 provides a representative TEM image of empty AA-T3A-C12 LNP. Scale bar, 100 nm.

FIG. 14 depicts hydrodynamic sizes of AA-T3A-C12/siRNA LNP. 1% bovine serum albumin (BSA) and 10/a FBS were prepared in 1×PBS and filtered through 0.22 μm filters to remove large protein aggregates. LNPs were then diluted in 1×PBS, 1% BSA/PBS or 10% FBS/PBS and incubated at the indicated temperature for 48 h. The average hydrodynamic size was determined by DLS. The absorption of protein on LNPs increased the size, but large LNP aggregates (>200 nm) were not observed. Data are presented as mean±SD (n=3).

FIG. 15 depicts flow cytometry analysis of cellular uptake of Cy5-siRNA-loaded AA-T3A-C12 LNP in 3T3 fibroblasts with or without TGF-β stimulation. Data are presented as mean±SD (n=3). ***p<0.001.

FIG. 16 depicts the flow cytometry gating strategy of FIG. 12C. GFP-positive 3T3-GFP fibroblasts and GFP-negative H2.35 hepatocytes were gated to obtain Cy5-siRNA signal inside cells.

FIG. 17 depicts flow cytometry analysis of competitive cellular uptake of Cy5-siRNA-loaded AA-T3A-C12 LNP or T3A-C12 LNP in a fibroblast/hepatocyte (3T3-GFP/H2.35) co-culture environment. The mean fluorescence intensity ratio between fibroblast and hepatocyte (MFI_{3T3-GFP}/MFI_{H2.35}) was calculated to indicate the preferential uptake of LNPs by fibroblasts over hepatocytes. The MFI_{3T3-GFP}/MFI_{H2.35} of AA-T3A-C12 LNP was 0.56, which was significantly higher than 0.36 of T3A-C12 LNP. Data are presented as mean±SD (n=3). **p<0.01; ***p<0.001.

FIGS. 18A-18B depict in vitro luciferase (luc) mRNA delivery by LNPs. FIG. 18A: AA-T3A-C12 LNP- and MC3 LNP-mediated luc mRNA delivery in activated 3T3 fibroblasts and H2.35 hepatocytes. 15 ng of luc mRNA was used to treat 5,000 cells per well for 24 h. FIG. 18B: Cell viability at 24 h post-treatment. Data are presented as mean±SD (n=3). *p<0.05.

FIGS. 19A-19E depict AA-T3A-C12 LNP-mediated GFP and HSP47 knockdown in activated fibroblasts. FIG. 19A: GFP knockdown using AA-T3A-C12/siGFP LNP. Activated 3T3-GFP fibroblasts were treated with AA-T3A-C12/siGFP LNP at the indicated dose for 24 or 48 h. FIG. 19B: flow cytometry analysis of GFP expression after AA-T3A-C12/siGFP LNP treatment for 48 h. FIG. 19C: immunofluorescence (IF) staining of HSP47 in LNP-treated activated 3T3 fibroblasts. Scale bar: 20 μm. FIGS. 19D-19E: western blot analysis of HSP47 expression in LNP-treated activated 3T3 fibroblasts (FIG. 19D) and primary HSCs (FIG. 19E). GAPDH was used as an internal control. Quantitative analysis was performed using ImageJ software. Data are presented as mean±SD (n=3). **p<0.01; ***p<0.001.

FIG. 20 depicts cell viability after treatment with AA-T3A-C12/siGFP LNP for 48 h. Cell viability was above or approximately 80% at the siRNA dose tested. Data are presented as mean SD (n=3).

FIG. 21 depicts Western blot analysis of HSP47 expression in different cell types. Activated 3T3 cells and HSCs were obtained by stimulation with 10 ng/ml of TGF-β for 24 h. GAPDH was used as an internal control. HSP47 expression was normalized to endothelial cells. HSP47 expression was much higher in fibroblasts (primary HSCs and 3T3 cells) than other cell types. Moreover, activated fibroblasts further up-regulated HSP47 expression.

FIG. 22 depicts immunofluorescence staining of HSP47 in LNP-treated activated 3T3 fibroblasts. Scale bar: 100 μm.

FIG. 23 depicts Western blot analysis of sigma receptor expression in immortalized primary hepatocytes H2.35, primary HSCs and activated primary HSCs. Activated HSCs were obtained by stimulation with 10 ng/ml of TGF-β for 24 h. GAPDH was used as an internal control. The expression of sigma receptor was normalized to hepatocytes. The expression of sigma receptor was much higher in HSCs than hepatocytes. Moreover, activated HSCs further up-regulated sigma receptors.

FIGS. 24A-24F depict biodistribution and HSP47 silencing activity of LNPs in fibrotic mice. FIG. 24A: ex vivo fluorescence imaging and signal quantification of major organs from PBS, AA-T3A-C12 LNP/Cy5-siRNA or MC3 LNP/Cy5-siRNA treated fibrotic mice (n=3). FIG. 24B: scheme of CCl₄ and LNP treatment. Mice received intraperitoneal (i.p.) injections of 20% CCl₄ (0.7 μl/g) in corn oil twice a week for 4 weeks. LNPs were intravenously (i.v.) administrated at an siRNA dose of 5 μg/mouse twice weekly for 2 weeks. FIG. 24C: body weight changes of mice over time during the experiment (n=5). FIG. 24D: body weight at the end of experiment (n=5). FIG. 24E: IF staining of HSP47 in liver sections. Arrows indicate central veins. Quantitative analysis was performed using ImageJ software (n=5). Scale bar: 100 μm. FIG. 24F: Western blot analysis of HSP47 expression in liver lysates. GAPDH was used as an internal control. Representative images for two sets of mouse liver samples are shown. Quantitative analysis was performed using ImageJ software (n=3). Data are presented as mean±SD. G1, healthy mice; G2, PBS-treated fibrotic mice; G3, AA-T3A-C12/siGFP LNP-treated fibrotic mice; G4, AA-T3A-C12/siHSP47 LNP-treated fibrotic mice; G5, MC3/siHSP47 LNP-treated fibrotic mice. ns, not significant; *p<0.05; **p<0.01; ***p<0.001.

FIG. 25 depicts ex vivo fluorescence imaging and signal quantification of major organs from T3A-C12 LNP/Cy5-siRNA treated mice (n=3). Each mouse was i.v. injected with 5 μg of Cy5-siRNA. Data are presented as mean±SD.

FIG. 26 depicts representative confocal images of liver sections. Fibrotic mice were i.v. injected with AA-T3A-C12 LNP/Cy5-siRNA or MC3 LNP/Cy5-siRNA at an siRNA dose of 5 μg/mouse. 1 h post-injection, mice were euthanized and livers were collected for immunofluorescence staining. Compared to MC3 LNP, more AA-T3A-C12 LNP co-localized with or were close to HSCs (α-SMA+). Arrows indicate central veins of liver lobules. Scale bar: 100 μm.

FIG. 27 depicts Western blot analysis of HSP47 expression in the liver. Mice were i.p. injected with 20% CCl₄ (0.7 μl/g) in corn oil twice a week for 2 weeks and then i.v. injected with AA-T3A-C12/siHSP47 LNP at an siRNA dose of 5 μg/mouse (0.2 mg/kg) or 12.5 μg/mouse (0.5 mg/kg). Mice were euthanized 3 days after treatment and livers were harvested for western blot analysis. GAPDH was used as an internal control. Data are presented as mean±SD (n=3).

FIGS. 28A-28E depict macroscopic, histopathological and biochemical analysis of liver fibrosis. FIG. 28A: representative images of livers and liver sections stained with hematoxylin and eosin (H&E) or Picrosirius red. Black arrows indicate apoptotic hepatocytes. FIG. 28B: morphometric quantification of Picrosirius red stained areas by ImageJ software. FIG. 28C: quantification of serum alanine aminotransferase (ALT). FIG. 28D: quantification of serum aspartate aminotransferase (AST). FIG. 28E: quantification of serum total bilirubin (TBIL). Data are presented as mean±SD (n=5). ns, not significant; *p<0.05; ***p<0.001.

FIGS. 29A-29B depict normalized serum proinflammatory cytokine levels to healthy mice; IL-6 (FIG. 29A) and TNF-α (FIG. 29B). Data are presented as mean±SD (n=5). ns, not significant; *p<0.05.

FIG. 30 depicts H&E staining of hearts, spleens, lungs and kidneys from each group of mice. No histological abnormality was observed for these organs.

FIG. 31 depicts representative results demonstrating HSP47 silencing by LNPs comprising AA-T3A-C12 lipidoid (flow) using simulated NIH3T3, 50 nM siRNA for 48 h, stained with anti-HSP47 primary Ab and AF488-labeled secondary Ab.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Description

Liver fibrosis is an abnormal wound-healing response to chronic liver injury that affects millions of people worldwide and can further progress to liver cirrhosis and hepatocellular carcinoma, yet there are currently no approved antifibrotic therapies. Liver fibrosis is characterized by excessive accumulation of collagen-rich extracellular matrix (ECM). Hepatic stellate cells (HSCs), a liver-resident fibroblast population located in the space between liver sinusoidal endothelial cells (LSECs) and hepatocytes, are the main effector cells during liver fibrogenesis. Upon liver injury, quiescent HSCs become activated and trans-differentiated into proliferative, profibrogenic and contractile myofibroblasts that secrete excessive ECM components, especially collagen. Heat shock protein 47 (HSP47) is a collagen-specific molecular chaperone that plays a crucial role in the proper folding, assembly and secretion of collagen into the extracellular space. Hepatic expression of HSP47 is significantly increased in fibrotic liver, and activated HSCs are identified as the primary source of HSP47, indicating that overexpression of HSP47 in activated HSCs accelerates the progress of this disease by supporting collagen biogenesis. Thus, HSP47 represents a promising target for anti-fibrotic therapy. With no specific HSP47 inhibitors available, utilizing small interfering RNA (siRNA) to silence HSP47 represents an attractive therapeutic strategy to reduce collagen production and alleviate fibrosis. However, targeted and potent delivery of siRNA to activated HSCs remains challenging.

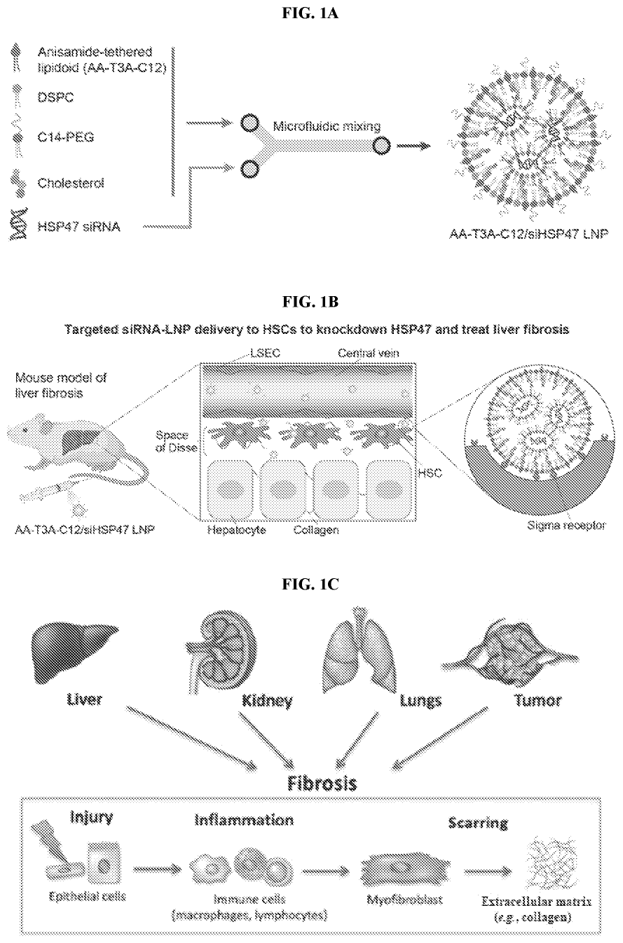

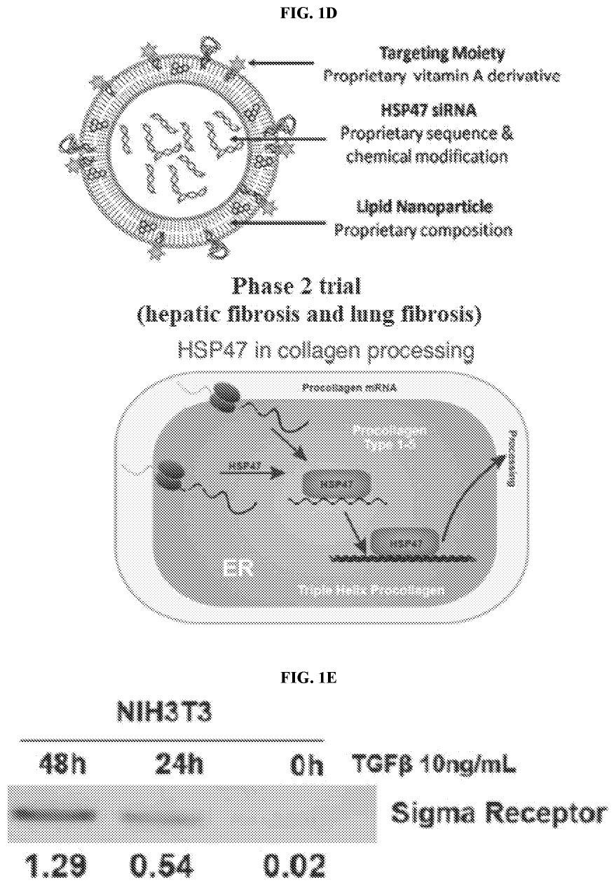

siRNA therapeutics face many extracellular and intracellular barriers, which necessitates the need of a delivery platform to achieve their potent delivery. Lipid nanoparticles (LNPs) are the most clinically advanced non-viral nucleic acid delivery platform, with the successful translation of one siRNA therapeutic and two mRNA vaccines. LNPs are multi-component systems that typically comprise of ionizable lipid (or lipidoid), cholesterol, phospholipid, and polyethylene glycol (PEG)-lipid (FIG. 1A). The cholesterol and phospholipid contribute to the stability of LNPs and facilitate membrane fusion. The PEG-lipid with short acyl chains stabilizes LNPs during formulation and storage, but detaches from LNPs rapidly to promote cellular uptake upon intravenous injection. The lipidoid is a key component to protect RNA therapeutics and drive endosomal escape for successful cytosolic delivery. In addition, the lipidoid greatly influences LNP tropism and transfected cell types in vivo. For example, by using different lipidoids, LNPs have successfully delivered RNA therapeutics to several types of liver cells, including hepatocytes, LSECs and Kupffer cells. Lipidoids that intrinsically mediate targeted RNA delivery to activated HSCs have not been reported, although some LNPs have been demonstrated to passively target these cells.

It was hypothesized that incorporating a small molecule ligand with affinity for activated HSCs into the lipidoid molecule itself could enable targeted siRNA-LNP delivery to these cells, as the rapid shedding of PEG in circulation will expose multivalent ligands on the surface of LNPs that can strongly bind with overexpressed receptors and mediate cellular uptake (FIG. 1B). Among various ligands that have been successfully used for HSC-targeted drug delivery, the neutral and stable anisamide moiety was selected, for the present studies, as the building block to construct anisamide-tethered lipidoids (AA-lipidoids). Notably, anisamide is a high-affinity ligand for the sigma receptor that is highly expressed on rapidly proliferating activated fibroblasts, including activated HSCs. In this study, a one-pot, two-step synthetic method was developed to enable parallel synthesis of a combinatorial library of AA-lipidoids. After two rounds of screening, the top-performing AA-lipidoid was identified (i.e., AA-T3A-C12), with both high potency and selectivity for activated fibroblasts transfection. In a mouse model of carbon tetrachloride (CCl₄)-induced liver fibrosis, HSP47 siRNA (siHSP47)-loaded AA-T3A-C12 LNP achieved 65% knockdown and dramatically reduced liver fibrosis, which significantly outperformed the benchmark DLin-MC3-DMA (MC3) LNP

These results demonstrate the potential of AA-lipidoids for targeted RNA delivery to activated fibroblasts. Furthermore, these synthetic methods and screening strategies open a new avenue to develop and discover potent lipidoids with targeting properties, which can potentially enable RNA delivery to a range of cell and tissue types that are challenging to access using traditional LNP formulations

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “about” as used herein can allow for a degree of variability in a value or range, for example, within 10%, within 5%, or within 1% of a stated value or of a stated limit of a range, and includes the exact stated value or range.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “adjuvant” as used herein is defined as any molecule to enhance an antigen-specific adaptive immune response.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)=CH₂, —C(CH₃)═CH(CH₃), —C(CH₂CH₃)=CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkylcarbonyl” refers to the —C(═O)Rₐ moiety, wherein Rₐ is an alkyl, alkenyl or alkynyl group as defined herein. A non-limiting example of an alkyl carbonyl is the methyl carbonyl (i.e., acetal) moiety. Alkylcarbonyl groups can also be referred to as “C_{w}-C_{z} acyl” where w and z depicts the range of the number of carbon in Rₐ, as defined above. For example, “C₁-C₁₀ acyl” refers to alkylcarbonyl group as defined above, where Rₐ is C₁-C₁₀ alkyl, C₁-C₁₀ alkenyl, or C₁-C₁₀ alkynyl group as defined above. Unless stated otherwise specifically in the specification, an alkyl carbonyl group can be optionally substituted.

The term “alkylene” or “alkylene chain” as used herein refers to a fully saturated, straight or branched divalent hydrocarbon, and having from 1 to 20 carbon atoms, and which has two points of attachment to the rest of the molecule. In some embodiments, the alkylene is a C₁-C₂₀ alkylene, a C₂-C₁₂ alkylene, a C₁-C₁₀ alkylene, a C₁-C₈ alkylene, a C₁-C₆ alkylene, a C₁-C₄ alkylene, or a C₁-C₃ alkylene. Non-limiting examples of C₁-C₂₀ alkylene include methylene, ethylene, propylene, n-butylene, ethenylene, propenylene, n-butenylene, propynylene, n-butynylene, and the like. The points of attachment of the alkylene chain to the rest of the molecule can be through one carbon or any two carbons within the chain. Unless stated otherwise specifically in the specification, an alkylene chain can be optionally substituted.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aralkenyl” or “arylalkenyl” as used herein refers to a radical of the formula —R_{b}—R_{c} where R_{b} is an alkenylene o group as defined above and R_{c} is one or more aryl radicals as defined herein. Unless stated otherwise specifically in the specification, an aralkenyl group can be optionally substituted.

The term “aralkynyl” or “arylalkynyl” as used herein refers to a radical of the formula —R_{b}—R_{c} where R_{b} is an alkynylene group as defined above and R_{c} is one or more aryl radicals as defined herein. Unless stated otherwise specifically in the specification, an aralkynyl group can be optionally substituted.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cat-ionic lipids comprise a protonatable tertiary amine (e.g., pH titratable) head group, Cis alkyl chains, ether linkages between the head group and alkyl chains, and 0 to 3 double bonds. Such lipids include, e.g., DSDMA, DLinDMA, DLenDMA, and DODMA.

The term “compound,” as used herein, unless otherwise indicated, refers to any specific chemical compound disclosed herein. In certain embodiments, the term also refers to stereoisomers and/or optical isomers (including racemic mixtures) or enantiomerically enriched mixtures of disclosed compounds.

The term “conjugated lipid” as used herein refers to a lipid which is conjugated to one or more polymeric groups, which inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

The term “cyano” refers to a group of the formula —CN group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “cycloalkenyl” as used herein refers to a stable non aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

The term “cycloalkynyl” as used herein refers to a stable non aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyls include, for example, cycloheptynyl, cyclooctynyl, and the like. Unless otherwise stated specifically in the specification, a cycloalkynyl group can be optionally substituted.

The term “cycloalkylalkyl” as used herein refers to a radical of the formula —R_{b}—R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “expression vector” as used herein refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) RNA, and viruses (e.g., lentiviruses, retroviruses, adenoviruses, and adeno-associated viruses) that incorporate the recombinant polynucleotide.

As used herein, “fibrosis” refers to the formation of excess fibrous connective tissue as a result of the excess deposition of extracellular matrix components, for example collagen. Fibrous connective tissue is characterized by having extracellular matrix (ECM) with a high collagen content. The collagen may be provided in strands or fibers, which may be arranged irregularly or aligned. The ECM of fibrous connective tissue may also include glycosaminoglycans. As used herein, the term “fibrosis” also refers to those diseases/conditions associated with, or characterized by, fibrosis. Examples include, but are not limited to, respiratory conditions such as pulmonary fibrosis, cystic fibrosis, idiopathic pulmonary fibrosis, progressive massive fibrosis, scleroderma, obliterative bronchiolitis, Hermansky-Pudlak syndrome, asbestosis, silicosis, chronic pulmonary hypertension, AIDS associated pulmonary hypertension, sarcoidosis, tumor stroma in lung disease, and asthma; chronic liver disease, primary biliary cirrhosis (PBC), schistosomal liver disease, liver cirrhosis; cardiovascular conditions such as hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts; neurological conditions such as gliosis and Alzheimer's disease; muscular dystrophy such as Duchenne muscular dystrophy (DMD) or Becker's muscular dystrophy (BMD); gastrointestinal conditions such as Chron's disease, microscopic colitis and primary sclerosing cholangitis (PSC); skin conditions such as scleroderma, nephrogenic systemic fibrosis and cutis keloid; arthrofibrosis; Dupuytren's contracture; mediastinal fibrosis; retroperitoneal fibrosis; myelofibrosis; Peyronie's disease; adhesive capsulitis; kidney disease (e.g., renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS); chronic graft versus host disease; diseases of the eye such as Grave's ophthalmopathy, epiretinal fibrosis, retinal fibrosis, subretinal fibrosis (e.g. associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis; arthritis; fibrotic pre-neoplastic and fibrotic neoplastic disease; and fibrosis induced by chemical or environmental insult (e.g., cancer chemotherapy, pesticides, radiation/cancer radiotherapy).

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The term “helper lipid” as used herein refers to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), 1-palmitoyl-2-oleoyl-sn-glycero-3phosphocholin (POPC) and 1,2-dioleoyl-sn-glycero-3-phosphocholine (DOPC).e in the animal's state of health.

The term “heteroarylalkyl” as used herein refers to a radical of the formula —R_{b}—R_{f} where R_{b} is an alkylene chain as defined above and R_{f} is a heteroaryl radical as defined herein. Unless stated otherwise specifically in the specification, a heteroarylalkyl group can be optionally substituted.

The term “heteroarylalkenyl” as used herein refers to a radical of the formula —R_{b}—R_{f} where R_{b} is an alkenylene, chain as defined above and R_{f} is a heteroaryl radical as defined herein Unless stated otherwise specifically in the specification, a heteroarylalkenyl group can be optionally substituted.

The term “heteroarylalkynyl” as used herein refers to a radical of the formula —R_{b}—R_{f} where R_{b} is an alkynylene chain as defined above and R_{f} is a heteroaryl radical as defined herein. Unless stated otherwise specifically in the specification, a heteroarylalkynyl group can be optionally substituted.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroalkyl” as used herein, by itself or in combination with another term means, unless stated otherwise, a stable straight or branched chain alkyl group consisting of 1 to carbon atoms and one or two heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as to the most distal carbon atom in the heteroalkyl group. Non-limiting examples include —OCH₂CH₂CH₃, —CH₂CH₂CH₂OH, —CH₂CH₂NHCH₃, —CH₂SCH₂CH₃, and —CH₂CH₂—S(═O)—CH₃. Up to two heteroatoms may be consecutive, such as, for example, —CH₂NHOCH₃ or —CH₂CH₂S—S—CH₃. Unless stated otherwise specifically in the specification, a heteroalkyl group can be optionally substituted.

“Homologous” as used herein, refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “hydroxy” or “hydroxyl” refers to a group of the formula —OH.

The term “imino” refers to a group of the formula ═NH group.

The term “immunogen” as used herein refers to any substance introduced into the body in order to generate an immune response. That substance can a physical molecule, such as a protein, or can be encoded by a vector, such as DNA, mRNA, or a virus.

The term “immune cell,” as used herein, means any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKa of the protonatable group in the range of about 4 to about 7.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “isomers” or “stereoisomers” as used herein refers to compounds which have identical chemical constitution (i.e., atoms and bonds), but which differ with regard to the arrangement of the atoms or groups in space.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “nitro” refers to a group of the formula ═O group.

The term “non-cationic lipid” refers to any amphipathic lipid as well as any other neutral lipid or anionic lipid.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns. In addition, the nucleotide sequence may contain modified nucleosides that are capable of being translation by translational machinery in a cell. For example, an mRNA where all of the uridines have been replaced with pseudouridine, 1-methyl psuedouridien, or another modified nucleoside.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA or RNA sequences are contiguous and, where necessary to join two protein coding regions, in the same reading frame.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

In certain instances, the polynucleotide or nucleic acid of the invention is a “nucleoside-modified nucleic acid,” which refers to a nucleic acid comprising at least one modified nucleoside. A “modified nucleoside” refers to a nucleoside with a modification. For example, over one hundred different nucleoside modifications have been identified in RNA (Rozenski, et al., 1999, The RNA Modification Database: 1999 update. Nucl Acids Res 27: 196-197).

In certain embodiments, “pseudouridine” refers, In some embodiments, to m¹acp³Ψ (1-methyl-3-(3-amino-3-carboxypropyl) pseudouridine. In some embodiments, the term refers to m¹Ψ (1-methylpseudouridine). In some embodiments, the term refers to Ψm (2′-O-methylpseudouridine. In some embodiments, the term refers to m⁵D (5-methyldihydrouridine). In some embodiments, the term refers to m³Ψ (3-methylpseudouridine). In some embodiments, the term refers to a pseudouridine moiety that is not further modified. In some embodiments, the term refers to a monophosphate, diphosphate, or triphosphate of any of the above pseudouridines. In some embodiments, the term refers to any other pseudouridine known in the art. Each possibility represents a separate embodiment of the present invention.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “prodrug” refers to an agent that is converted into the parent drug in vivo. For example, the term “prodrug” refers to a derivative of a known direct acting drug, which derivative has enhanced delivery characteristics and therapeutic value as compared to the drug, and is transformed into the active drug by an enzymatic or chemical process. In some embodiments, “prodrug” refers to an inactive or relatively less active form of an active agent that becomes active by undergoing a chemical conversion through one or more metabolic processes. In certain embodiments, upon in vivo administration, a prodrug is chemically converted to the biologically, pharmaceutically, or therapeutically active form of the compound. In another embodiment, a prodrug is enzymatically metabolized by one or more steps or processes to the biologically, pharmaceutically, or therapeutically active form of the compound. For example, the present compounds can be administered to a subject as a prodrug that includes an initiator bound to an active agent, and, by virtue of being degraded by a metabolic process, the active agent is released in its active form.

The term “promoter” as used herein is defined as a DNA sequence recognized by the synthetic machinery of the cell, or introduced synthetic machinery, required to initiate the specific transcription of a polynucleotide sequence. For example, the promoter that is recognized by bacteriophage RNA polymerase and is used to generate the mRNA by in vitro transcription.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl

By the term “synthetic antibody” as used herein, is meant an antibody, which is generated using recombinant DNA technology, such as, for example, an antibody expressed by a bacteriophage. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art. The term should also be construed to mean an antibody, which has been generated by the synthesis of an RNA molecule encoding the antibody. The RNA molecule expresses an antibody protein, or an amino acid sequence specifying the antibody, wherein the RNA has been obtained by transcribing DNA (synthetic or cloned) or other technology, which is available and well known in the art.

The term “tautomers” are constitutional isomers of organic compounds that readily interconvert by a chemical process (i.e., tautomerization).

The term “therapeutic” as used herein means a treatment and/or prophylaxis. A therapeutic effect is obtained by suppression, diminution, remission, or eradication of at least one sign or symptom of a disease or disorder state.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Selected Lipidoid Compounds

In one aspect, the present disclosure provides a compound of Formula (I), or a salt, solvate, stereoisomer, tautomer, or isotopologue thereof:

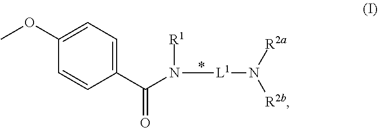

wherein:
L¹ is selected from the group consisting of -(optionally substituted C₂-C₁₂ heteroalkylenyl)- and -(optionally substituted C₂-C₁₂ heteroalkylenyl)-(optionally substituted C₂-C₁₂ heterocycloalkylenyl)-,
   wherein each occurrence of C₂-C₁₂ heteroalkylenyl and C₂-C₁₂ heterocycloalkylenyl is optionally substituted with at least one substituent selected from the group consisting of optionally substituted C₁-C₂₄ alkyl, optionally substituted C₁-C₂₄ heteroalkyl optionally substituted C₃-C₈ cycloalkyl, optionally substituted C₆-C₁₀ aryl, optionally substituted C₂-C₁₀ heteroaryl, R^{2c}, and

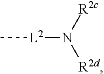

or two geminal substituents may combine to form ═O;
each occurrence of L² is independently selected from the group consisting of -(optionally substituted C₁-C₆ alkylenyl)-, -(optionally substituted C₂-C₆ heteroalkylenyl)-, -(optionally substituted C₁-C₆ alkylenyl)-C(═O)X-(optionally substituted C₁-C₆ alkylenyl)-, and -(optionally substituted C₁-C₆ alkylenyl)-XC(═O)-(optionally substituted C₁-C₆ alkylenyl)-;
R¹ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{2a}, R^{2b}, R^{2c}, and R^{2d}, if present, is independently selected from the group consisting of H, -(optionally substituted C₁-C₆ alkylenyl)-C(═O)OR³, -(optionally substituted C₁-C₆ alkylenyl)-C(═O)N(R³)(R⁴), -(optionally substituted C₁-C₆ alkylenyl)-C(═O)R³, -(optionally substituted C₁-C₆ alkylenyl)-(R³), —C(═O)OR³, —C(═O)N(R³)(R⁴), —C(═O)R³, and R³,
   wherein no more than one of each occurrence of R^{2a}, R^{2b}, R^{2c}, and R^{2d} is H;
R³ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁴ is selected from the group consisting of H and optionally substituted C₁-C₆ alkyl; each occurrence of X is independently selected from the group consisting of a bond, O, and NR⁵; and
each occurrence of R⁵ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

In certain embodiments, R¹ is H.

In certain embodiments, L¹ is selected from the group consisting of:

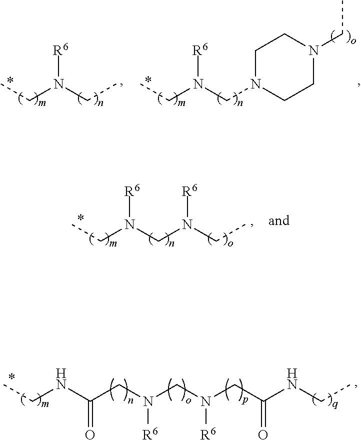

wherein:
each occurrence of R⁶ is independently selected from the group consisting of H, optionally substituted C₁-C₂₄ alkyl, and

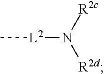

m, n, o, p, and q are each independently an integer selected from the group consisting of 1, 2, 3, and 4.

In certain embodiments, R⁶ is H. In certain embodiments, R⁶ is CH₃. In certain embodiments, R⁶ is CH₂CH(OH)(optionally substituted C₁-C₁₂ alkyl). In certain embodiments, R⁶ is

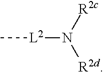

In certain embodiments, L² is —CH₂CH₂—. In certain embodiments, L² is —CH₂CH₂CH₂—. In certain embodiments, L² is —CH₂CH₂—C(═O)NH—CH₂CH₂—.

In certain embodiments, L¹ is

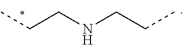

In certain embodiments, L¹ is

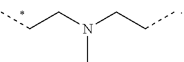

In certain embodiments, L¹ is

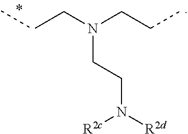

In certain embodiments, L¹ is

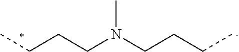

In certain embodiments, L¹ is

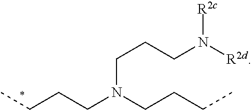

In certain embodiments, L¹ is

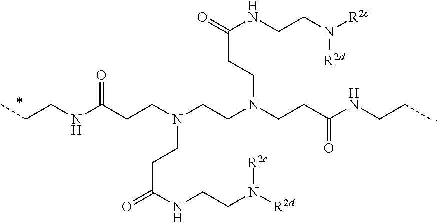

In certain embodiments, L¹ is

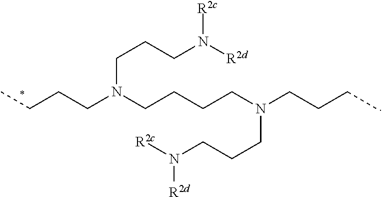

In certain embodiments, R^{2a} is H. In certain embodiments, R^{2a} is —CH₂CH(OH)(optionally substituted C₁-C₂₈ alkyl). In certain embodiments, R^{2a} is —CH₂CH(OH)(optionally substituted C₂-C₂₈ alkenyl). In certain embodiments, R^{2a} is —CH₂CH₂C(═O)O(optionally substituted C₁-C₂₈ alkyl). In certain embodiments, R^{2a} is —CH₂CH₂C(═O)NH(optionally substituted C₁-C₂₈ alkyl).

In certain embodiments, R^{2b} is H. In certain embodiments, R^{2b} is —CH₂CH(OH)(optionally substituted C₁-C₂₈ alkyl). In certain embodiments, R^{2b} is —CH₂CH(OH)(optionally substituted C₂-C₂₈ alkenyl). In certain embodiments, R^{2b} is —CH₂CH₂C(═O)O(optionally substituted C₁-C₂₈ alkyl). In certain embodiments, R^{2b} is —CH₂CH₂C(═O)NH(optionally substituted C₁-C₂₈ alkyl).

In certain embodiments, R^{2c} is H. In certain embodiments, R^{2c} is —CH₂CH(OH)(optionally substituted C₁-C₂₈ alkyl). In certain embodiments, R^{2c} is —CH₂CH(OH)(optionally substituted C₂-C₂₈ alkenyl). In certain embodiments, R^{2c} is —CH₂CH₂C(═O)O(optionally substituted C₁-C₂₈ alkyl). In certain embodiments, R^{2c} is —CH₂CH₂C(═O)NH(optionally substituted C₁-C₂₈ alkyl). In certain embodiments, R^{2d} is H. In certain embodiments, R^{2d} is —CH₂CH(OH)(optionally substituted C₁-C₂₈ alkyl). In certain embodiments, R^{2d} is —CH₂CH(OH)(optionally substituted C₂-C₂₈ alkenyl). In certain embodiments, R^{2d} is —CH₂CH₂C(═O)O(optionally substituted C₁-C₂₈ alkyl). In certain embodiments, R^{2d} is —CH₂CH₂C(═O)NH(optionally substituted C₁-C₂₈ alkyl).

In certain embodiments, R^{2a} is —CH₂CH(OH)(CH₂)₉CH₃. In certain embodiments, R^{2a} is —CH₂CH(OH)(CH₂)₁₁CH₃. In certain embodiments, R^{2a} is —CH₂CH(OH)(CH₂)₁₃CH₃.

In certain embodiments, R^{2b} is —CH₂CH(OH)(CH₂)₉CH₃. In certain embodiments, R^{2b} is —CH₂CH(OH)(CH₂)₁₁CH₃. In certain embodiments, R^{2b} is —CH₂CH(OH)(CH₂)₁₃CH₃.

In certain embodiments, R^{2c} is —CH₂CH(OH)(CH₂)₉CH₃. In certain embodiments, R^{2c} is —CH₂CH(OH)(CH₂)₁₁CH₃. In certain embodiments, R^{2c} is —CH₂CH(OH)(CH₂)₁₃CH₃.

In certain embodiments, R^{2d} is —CH₂CH(OH)(CH₂)₉CH₃. In certain embodiments, R^{2d} is —CH₂CH(OH)(CH₂)₁₁CH₃. In certain embodiments, R^{2d} is —CH₂CH(OH)(CH₂)₁₃CH₃.

In certain embodiments, R³ is H. In certain embodiments, R³ is —CH₂CH(OH)(optionally substituted C₁-C₂₈ alkyl). In certain embodiments, R³ is —CH₂CH(OH)(optionally substituted C₂-C₂₈ alkenyl). In certain embodiments, R³ is —CH₂CH₂C(═O)O(optionally substituted C₁-C₂₈ alkyl). In certain embodiments, R³ is —CH₂CH₂C(═O)NH(optionally substituted C₁-C₂₈ alkyl).

In certain embodiments, R³ is —CH₂CH(OH)(CH₂)₉CH₃. In certain embodiments, R³ is —CH₂CH(OH)(CH₂)₁₁CH₃. In certain embodiments, R³ is —CH₂CH(OH)(CH₂)₁₃CH₃.

In certain embodiments, R⁴ is H.

In certain embodiments, R⁵ is H.

In certain embodiments, X is NH.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, and optionally substituted heteroalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound is

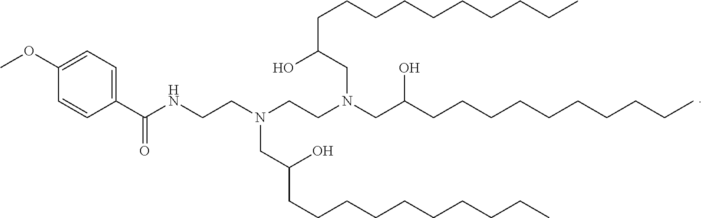 [SMILES: CCCCCCCCCCC(O)CN(CCNC(=O)c1ccc(OC)cc1)CCN(CC(O)CCCCCCCCCC)CC(O)CCCCCCCCCC]

In certain embodiments, the compound is

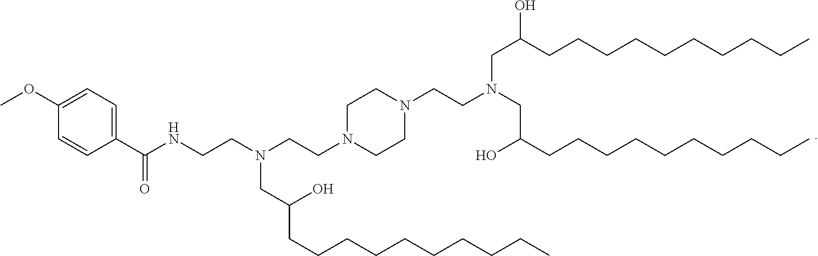 [SMILES: CCCCCCCCCCC(O)CN(CCNC(=O)c1ccc(OC)cc1)CCN1CCN(CCN(CC(O)CCCCCCCCCC)CC(O)CCCCCCCCCC)CC1]

In certain embodiments, the compound is

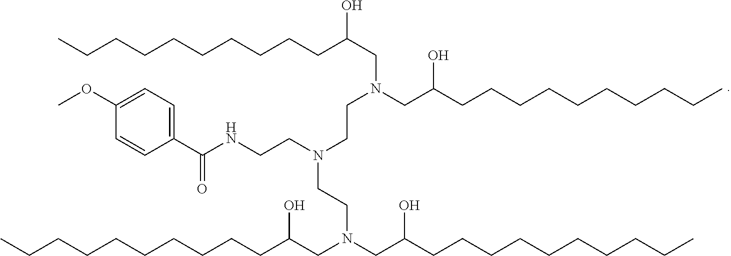 [SMILES: CCCCCCCCCCC(O)CN(CCN(CCNC(=O)c1ccc(OC)cc1)CCN(CC(O)CCCCCCCCCC)CC(O)CCCCCCCCCC)CC(O)CCCCCCCCCC]

In certain embodiments, the compound is

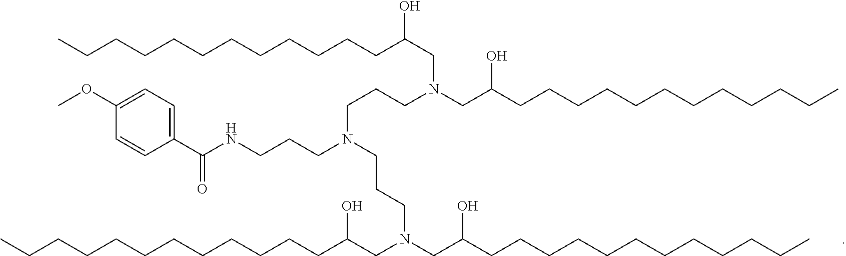 [SMILES: CCCCCCCCCCCCC(O)CN(CCCN(CCCNC(=O)c1ccc(OC)cc1)CCCN(CC(O)CCCCCCCCCCCC)CC(O)CCCCCCCCCCCC)CC(O)CCCCCCCCCCCC]

In certain embodiments, the compound is

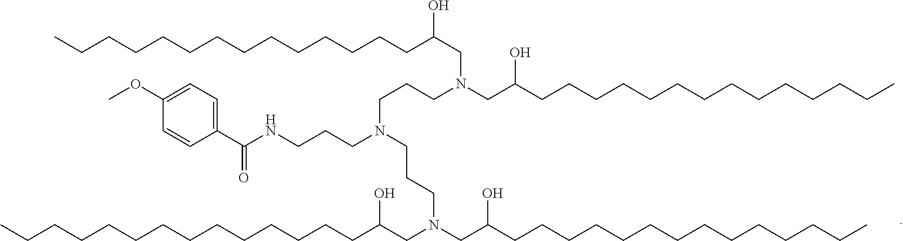 [SMILES: CCCCCCCCCCCCCCC(O)CN(CCCN(CCCNC(=O)c1ccc(OC)cc1)CCCN(CC(O)CCCCCCCCCCCCCC)CC(O)CCCCCCCCCCCCCC)CC(O)CCCCCCCCCCCCCC]

In certain embodiments, the compound is

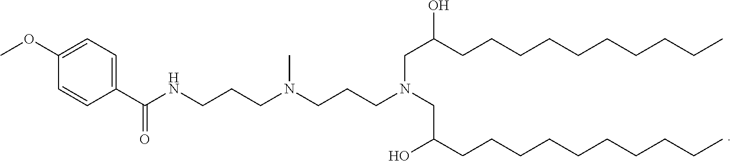 [SMILES: CCCCCCCCCCC(O)CN(CCCN(C)CCCNC(=O)c1ccc(OC)cc1)CC(O)CCCCCCCCCC]

In certain embodiments, the compound is

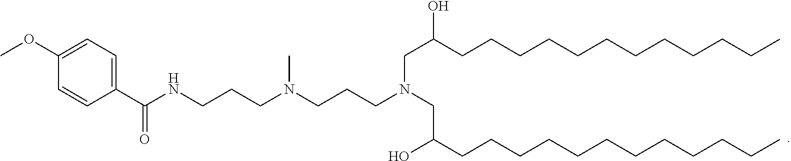 [SMILES: CCCCCCCCCCCCC(O)CN(CCCN(C)CCCNC(=O)c1ccc(OC)cc1)CC(O)CCCCCCCCCCCC]

In certain embodiments, the compound is

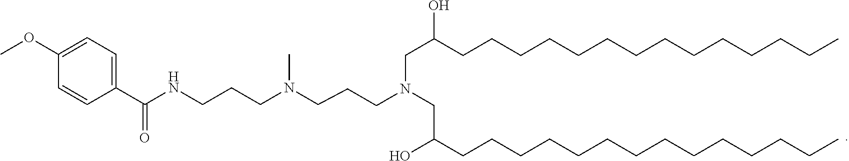 [SMILES: CCCCCCCCCCCCCCC(O)CN(CCCN(C)CCCNC(=O)c1ccc(OC)cc1)CC(O)CCCCCCCCCCCCCC]

In certain embodiments, the compound is

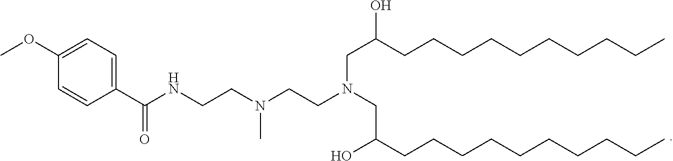 [SMILES: CCCCCCCCCCC(O)CN(CCN(C)CCNC(=O)c1ccc(OC)cc1)CC(O)CCCCCCCCCC]

In certain embodiments, the compound is

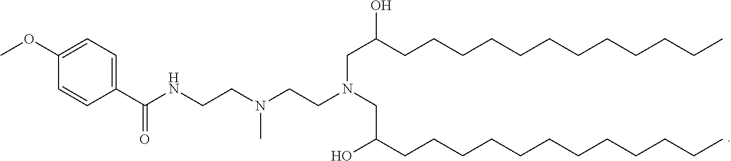 [SMILES: CCCCCCCCCCCCC(O)CN(CCN(C)CCNC(=O)c1ccc(OC)cc1)CC(O)CCCCCCCCCCCC]

In certain embodiments, the compound is

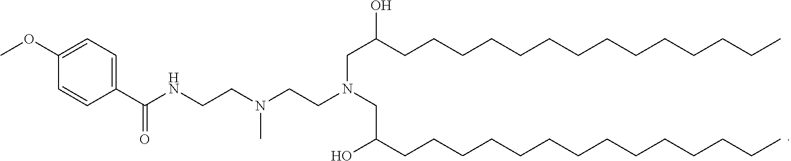 [SMILES: CCCCCCCCCCCCCCC(O)CN(CCN(C)CCNC(=O)c1ccc(OC)cc1)CC(O)CCCCCCCCCCCCCC]

In certain embodiments, the compound is

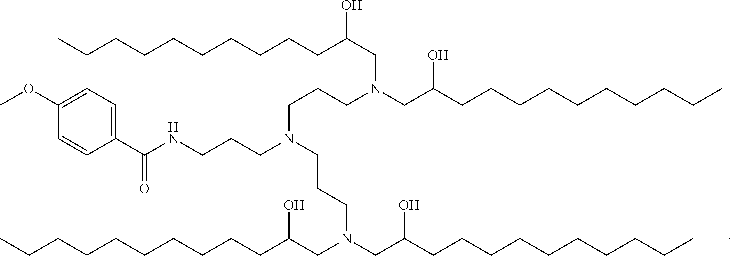 [SMILES: CCCCCCCCCCC(O)CN(CCCN(CCCNC(=O)c1ccc(OC)cc1)CCCN(CC(O)CCCCCCCCCC)CC(O)CCCCCCCCCC)CC(O)CCCCCCCCCC]

In certain embodiments, the compound is

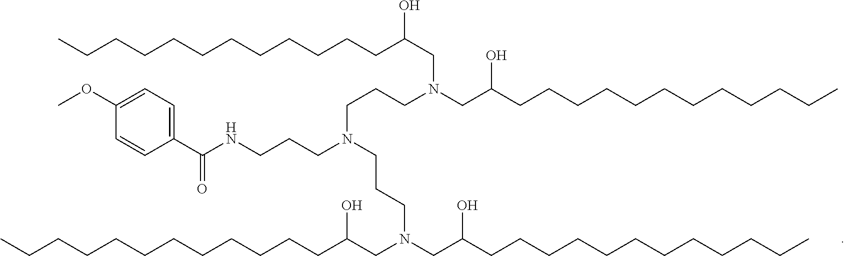 [SMILES: CCCCCCCCCCCCC(O)CN(CCCN(CCCNC(=O)c1ccc(OC)cc1)CCCN(CC(O)CCCCCCCCCCCC)CC(O)CCCCCCCCCCCC)CC(O)CCCCCCCCCCCC]

In certain embodiments, the compound is

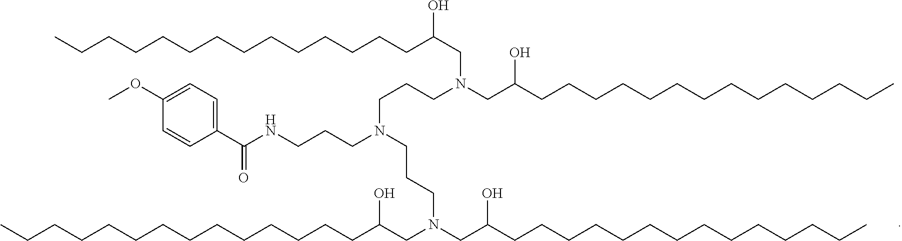 [SMILES: CCCCCCCCCCCCCCC(O)CN(CCCN(CCCNC(=O)c1ccc(OC)cc1)CCCN(CC(O)CCCCCCCCCCCCCC)CC(O)CCCCCCCCCCCCCC)CC(O)CCCCCCCCCCCCCC]

In certain embodiments, the compound is

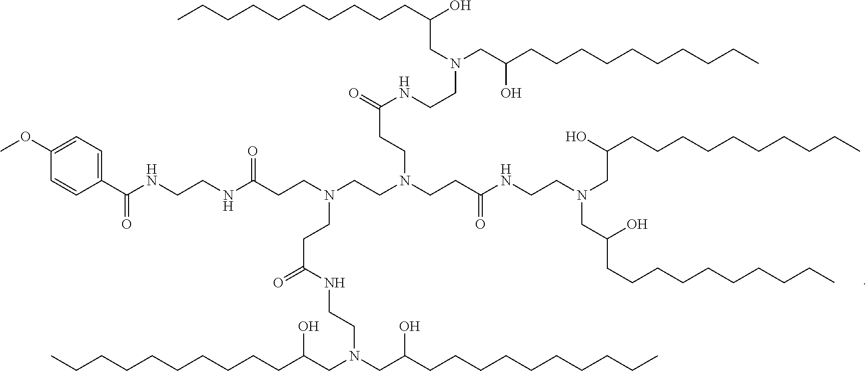 [SMILES: CCCCCCCCCCC(O)CN(CCNC(=O)CCN(CCC(=O)NCCNC(=O)c1ccc(OC)cc1)CCN(CCC(=O)NCCN(CC(O)CCCCCCCCCC)CC(O)CCCCCCCCCC)CCC(=O)NCCN(CC(O)CCCCCCCCCC)CC(O)CCCCCCCCCC)CC(O)CCCCCCCCCC]

In certain embodiments, the compound is

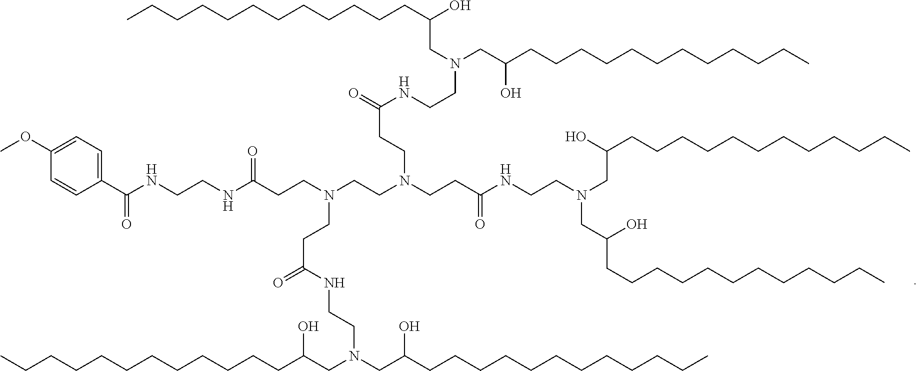 [SMILES: CCCCCCCCCCCCC(O)CN(CCNC(=O)CCN(CCC(=O)NCCNC(=O)c1ccc(OC)cc1)CCN(CCC(=O)NCCN(CC(O)CCCCCCCCCCCC)CC(O)CCCCCCCCCCCC)CCC(=O)NCCN(CC(O)CCCCCCCCCCCC)CC(O)CCCCCCCCCCCC)CC(O)CCCCCCCCCCCC]

In certain embodiments, the compound is

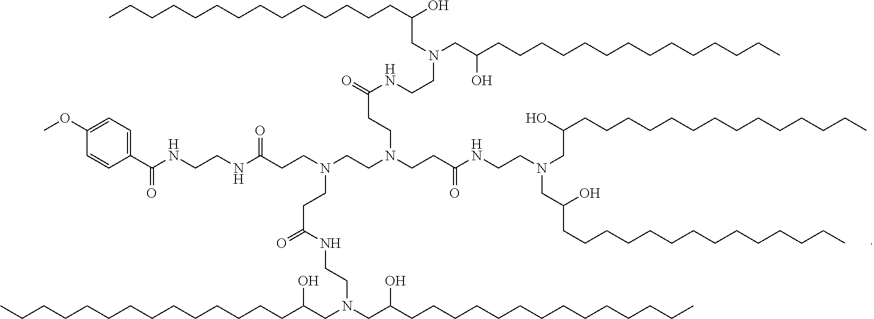 [SMILES: CCCCCCCCCCCCCCC(O)CN(CCNC(=O)CCN(CCC(=O)NCCNC(=O)c1ccc(OC)cc1)CCN(CCC(=O)NCCN(CC(O)CCCCCCCCCCCCCC)CC(O)CCCCCCCCCCCCCC)CCC(=O)NCCN(CC(O)CCCCCCCCCCCCCC)CC(O)CCCCCCCCCCCCCC)CC(O)CCCCCCCCCCCCCC]

In certain embodiments, the compound is

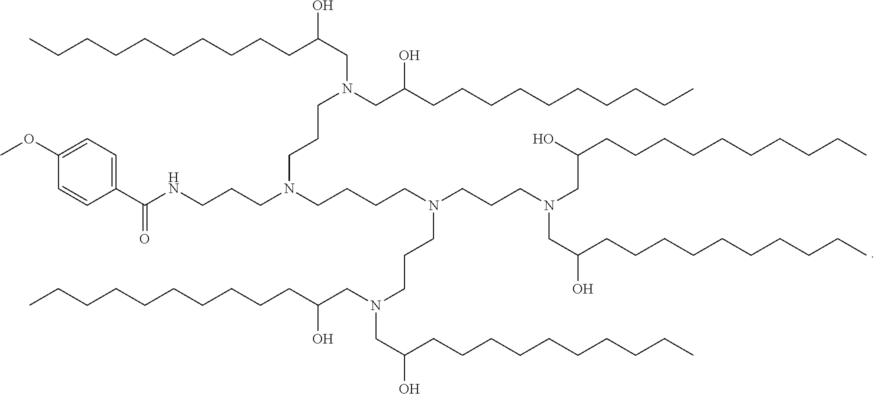 [SMILES: CCCCCCCCCCC(O)CN(CCCN(CCCCN(CCCN(CC(O)CCCCCCCCCC)CC(O)CCCCCCCCCC)CCCN(CC(O)CCCCCCCCCC)CC(O)CCCCCCCCCC)CCCNC(=O)c1ccc(OC)cc1)CC(O)CCCCCCCCCC]

In certain embodiments, the compound is

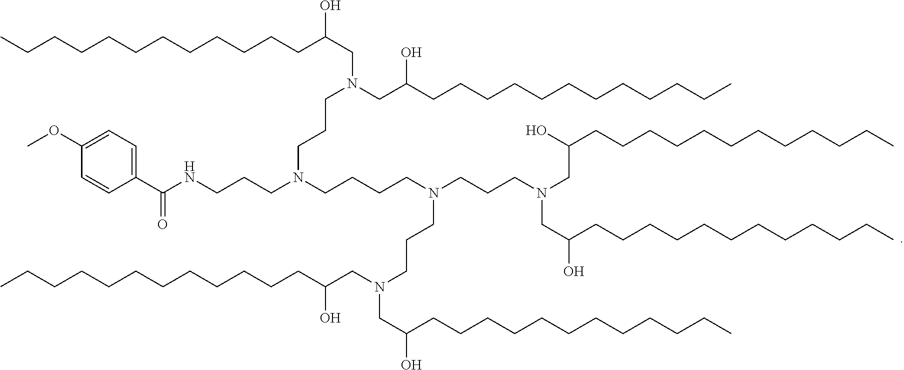 [SMILES: CCCCCCCCCCCCC(O)CN(CCCN(CCCCN(CCCN(CC(O)CCCCCCCCCCCC)CC(O)CCCCCCCCCCCC)CCCN(CC(O)CCCCCCCCCCCC)CC(O)CCCCCCCCCCCC)CCCNC(=O)c1ccc(OC)cc1)CC(O)CCCCCCCCCCCC]

In certain embodiments, the compound is

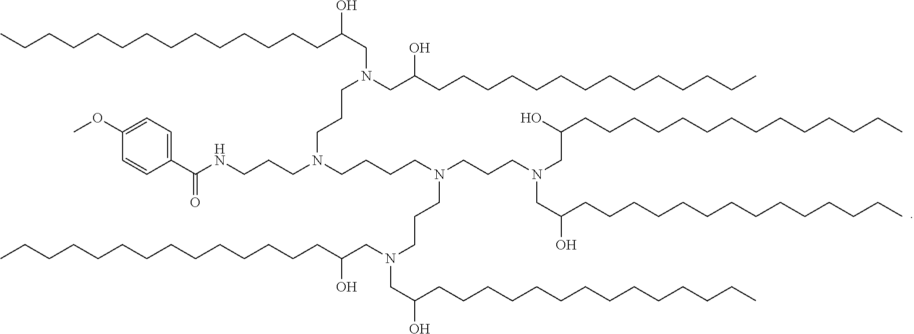 [SMILES: CCCCCCCCCCCCCCC(O)CN(CCCN(CCCCN(CCCN(CC(O)CCCCCCCCCCCCCC)CC(O)CCCCCCCCCCCCCC)CCCN(CC(O)CCCCCCCCCCCCCC)CC(O)CCCCCCCCCCCCCC)CCCNC(=O)c1ccc(OC)cc1)CC(O)CCCCCCCCCCCCCC]

### Selected Lipids

As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In certain embodiments, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

In certain embodiments, the lipid is a PEGylated lipid, including, but not limited to, DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid.

The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides.

Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), distearoyl-phosphatidylethanolamine (DSPE)-maleimide-PEG, distearoyl-phosphatidylethanolamine (DSPE)-maleimide-PEG2000, 16-O-monomethyl PE, 16-O-dimethyl PE, 18-1-trans PE, 1-stearioyl-2-oleoyl-phosphatidyethanol amine (SOPE), stearoyloleoylphosphatidylcholine (SOPC), and 1,2-dielaidoyl-sn-glycero-3-phophoethanolamine (transDOPE). In certain embodiments, the neutral lipid is 1,2-distearoyl-sn-glycero-3-phosphocholine (DSPC).

In some embodiments, the composition comprises a neutral lipid selected from DSPC, DPPC, DMPC, DOPC, POPC, DOPE, and SM.

A “steroid” is a compound comprising the following carbon skeleton:

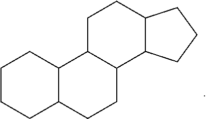 [SMILES: C1CCC2C(C1)CCC1C3CCCC3CCC21]

In certain embodiments, the steroid or steroid analogue is cholesterol. In some of these embodiments, the molar ratio of the cationic lipid.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “polymer conjugated lipid” or “conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include polyethylene glycol (PEG), maleimide PEG (mPEG), DSPE-PEG-DBCO, 1-(monomethoxy-polyethyleneglycol)-2,3-dimyristoylglycerol (PEG-s-DMG), DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

In certain embodiments, the LNP comprises an additional, stabilizing-lipid which is a polyethylene glycol-lipid (pegylated lipid). Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In certain embodiments, the polyethylene glycol-lipid is N-[(methoxy poly(ethylene glycol)₂₀₀₀)carbamyl]-1,2-dimyristyloxlpropyl-3-amine (PEG-c-DMA). In certain embodiments, the polyethylene glycol-lipid is PEG-c-DOMG). In other embodiments, the LNPs comprise a pegylated diacylglycerol (PEG-DAG) such as 1-(monomethoxy-polyethyleneglycol)-2,3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy)propyl-1-O—(ω-methoxy(polyethoxy)ethyl)butanedioate (PEG-S-DMG), a pegylated ceramide (PEG-cer), or a PEG dialkoxypropylcarbamate such as w-methoxy(polyethoxy)ethyl-N-(2,3-di(tetradecanoxy)propyl)carbamate or 2,3-di(tetradecanoxy)propyl-N—(ω-methoxy(polyethoxy)ethyl)carbamate.

In certain embodiments, the additional lipid is present in the LNP in an amount from about 1 mol % to about 10 mol %. In certain embodiments, the additional lipid is present in the LNP in an amount from about 1 mol % to about 5 mol %. In certain embodiments, the additional lipid is present in the LNP in about 1 mol % or about 2.5 mol %.

The term “lipid nanoparticle” refers to a particle having at least one dimension on the order of nanometers (e.g., 1-1,000 nm) which includes one or more lipids.

In various embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In various embodiments, the lipids or the LNP of the present invention are substantially non-toxic.

In various embodiments, the lipids or the LNPs described herein are formulated for stability for in vivo immune cell targeting.

In some embodiments, the LNP formulated for stability for in vivo immune cell targeting comprises C14-4 in a concentration range of about 10 mol % to about 45 mol %. In some embodiments, the C14-4 is present in a molar ratio of about 40%.

In some embodiments, the LNP formulated for stability for in vivo immune cell targeting comprises a phospholipid in a concentration range of about 10 mol % to about 45 mol %. In certain embodiments, the phospholipid is dioleoyl-phosphatidylethanolamine (DOPE), and the DOPE is present in a molar ratio of about 25 or at a molar percentage of about 25%.

In some embodiments, the LNP formulated for stability for in vivo immune cell targeting comprises a cholesterol lipid in a concentration range of about 5 mol % to about 50 mol %. In certain embodiments, the cholesterol is present in a molar ratio of about 30, or at a molar percentage of about 30%.

In some embodiments, the LNP formulated for stability for in vivo immune cell targeting comprises total PEG in a concentration range of about 0.5 mol % to about 12.5 mol %. In certain embodiments, the total PEG is present in a molar ratio of about 2.5, or at a molar percentage of about 2.5%.

In certain embodiments, the LNP formulated for stability for in vivo immune cell targeting comprises ionizable lipid C14-4, DOPE, cholesterol and total PEG, wherein the C14-4:DOPE:cholesterol:total PEG are present in a molar ratio of about 40:25:30:2.5 or at a molar percentage of about 40%:25%:30%:2.5%.

In some embodiments, the total PEG comprises maleimide PEG (mPEG) and PEG in a mol ratio of about 1:1, 1:2, 1:3, 1:4, 1:5, 1:6, 1:7, 1:8, 1:9, 1:10, 1:11, 1:12, 1:13, 1:14, 1:15 or greater than 1:15, or any molar ratio therebetween. In certain embodiments, the LNP comprises total PEG at a mol ratio of about 2.5, wherein the total PEG comprises mPEG and PEG at a mol ratio of 1:3. In certain embodiments, the LNP comprises total PEG at a mol ratio of about 2.5, and the total PEG comprises PEG and mPEG at a mol ratio of 1:5. In certain embodiments, the LNP comprises total PEG at a mol ratio of about 2.5, and the total PEG comprises PEG and mPEG at a mol ratio of 1:7. In certain embodiments, the LNP comprises total PEG at a mol ratio of about 2.5, and the total PEG comprises PEG and mPEG at a mol ratio of 1:10.

### Lipid Nanoparticle (LNP) Compositions

In another aspect, the present disclosure provides a lipid nanoparticle (LNP). In certain embodiments, the LNP comprises at least one ionizable lipidoid compound of Formula (I). In certain embodiments, the LNP comprises at least one helper lipid. In certain embodiments, the LNP comprises at least one cholesterol lipid. In certain embodiments, the LNP comprises at least one conjugated lipid.

In certain embodiments, the ionizable lipidoid compound of Formula (I), or a salt, solvate, stereoisomer, tautomer, or isotopologue thereof, has the following structure:

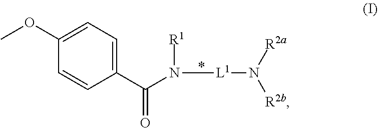

wherein:
L¹ is selected from the group consisting of -(optionally substituted C₂-C₁₂ heteroalkylenyl)- and -(optionally substituted C₂-C₁₂ heteroalkylenyl)-(optionally substituted C₂-C₁₂ heterocycloalkylenyl)-,
   wherein each occurrence of C₂-C₁₂ heteroalkylenyl and C₂-C₁₂ heterocycloalkylenyl is optionally substituted with at least one substituent selected from the group consisting of optionally substituted C₁-C₂₄ alkyl, optionally substituted C₁-C₂₄ heteroalkyl optionally substituted C₃-C₈ cycloalkyl, optionally substituted C₆-C₁₀ aryl, optionally substituted C₂-C₁₀ heteroaryl, R^{2c}, and

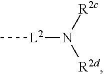

or two geminal substituents may combine to form ═O;
each occurrence of L² is independently selected from the group consisting of -(optionally substituted C₁-C₆ alkylenyl)-, -(optionally substituted C₂-C₆ heteroalkylenyl)-, -(optionally substituted C₁-C₆ alkylenyl)-C(═O)X-(optionally substituted C₁-C₆ alkylenyl)-, and -(optionally substituted C₁-C₆ alkylenyl)-XC(═O)-(optionally substituted C₁-C₆ alkylenyl)-;
R¹ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{2a}, R^{2b}, R^{2c}, and R^{2d}, if present, is independently selected from the group consisting of H, -(optionally substituted C₁-C₆ alkylenyl)-C(═O)OR³, -(optionally substituted C₁-C₆ alkylenyl)-C(═O)N(R³)(R⁴), -(optionally substituted C₁-C₆ alkylenyl)-C(═O)R³, -(optionally substituted C₁-C₆ alkylenyl)-(R³), —C(═O)OR³, —C(═O)N(R³)(R⁴), —C(═O)R³, and R³,
   wherein no more than one of each occurrence of R^{2a}, R^{2b}, R^{2c}, and R^{2d} is H;
R³ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁴ is selected from the group consisting of H and optionally substituted C₁-C₆ alkyl;
each occurrence of X is independently selected from the group consisting of a bond, O, and NR⁵; and
each occurrence of R⁵ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

In certain embodiments, R¹ is H.

In certain embodiments, L¹ is selected from the group consisting of:

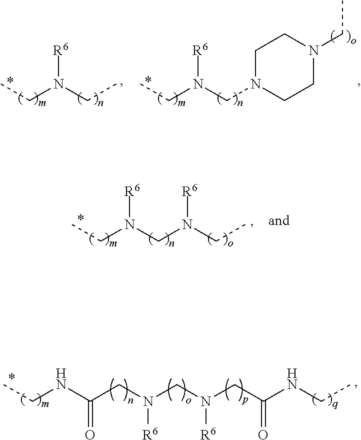

wherein:
each occurrence of R⁶ is independently selected from the group consisting of H, optionally substituted C₁-C₂₄ alkyl, and

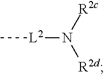

m, n, o, p, and q are each independently an integer selected from the group consisting of 1, 2, 3, and 4.

In certain embodiments, R⁶ is H. In certain embodiments, R⁶ is CH₃. In certain embodiments, R⁶ is CH₂CH(OH)(optionally substituted C₁-C₁₂ alkyl). In certain embodiments, R⁶ is

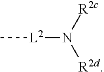

In certain embodiments, L² is —CH₂CH₂—. In certain embodiments, L² is —CH₂CH₂CH₂—. In certain embodiments, L² is —CH₂CH₂—C(═O)NH—CH₂CH₂—.

In certain embodiments, L¹ is

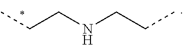

In certain embodiments, L¹ is

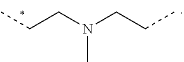

In certain embodiments, L¹ is

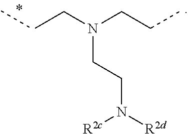

In certain embodiments, L¹ is

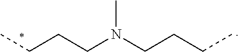

In certain embodiments, L¹ is

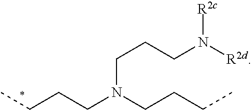

In certain embodiments, L¹ is

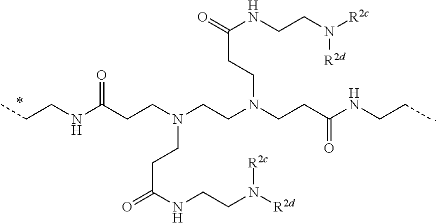

In certain embodiments, L¹ is

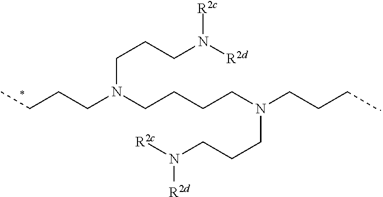

In certain embodiments, R^{2a} is H. In certain embodiments, R^{2a} is —CH₂CH(OH)(optionally substituted C₁-C₂₈ alkyl). In certain embodiments, R^{2a} is —CH₂CH(OH)(optionally substituted C₂-C₂₈ alkenyl). In certain embodiments, R^{2a} is —CH₂CH₂C(═O)O(optionally substituted C₁-C₂₈ alkyl). In certain embodiments, R^{2a} is —CH₂CH₂C(═O)NH(optionally substituted C₁-C₂₈ alkyl).

In certain embodiments, R^{2b} is H. In certain embodiments, R^{2b} is —CH₂CH(OH)(optionally substituted C₁-C₂₈ alkyl). In certain embodiments, R^{2b} is —CH₂CH(OH)(optionally substituted C₂-C₂₈ alkenyl). In certain embodiments, R^{2b} is —CH₂CH₂C(═O)O(optionally substituted C₁-C₂₈ alkyl). In certain embodiments, R^{2b} is —CH₂CH₂C(═O)NH(optionally substituted C₁-C₂₈ alkyl).

In certain embodiments, R^{2c} is H. In certain embodiments, R^{2c} is —CH₂CH(OH)(optionally substituted C₁-C₂₈ alkyl). In certain embodiments, R^{2c} is —CH₂CH(OH)(optionally substituted C₂-C₂₈ alkenyl). In certain embodiments, R^{2c} is —CH₂CH₂C(═O)O(optionally substituted C₁-C₂₈ alkyl). In certain embodiments, R^{2c} is —CH₂CH₂C(═O)NH(optionally substituted C₁-C₂₈ alkyl).

In certain embodiments, R^{2d} is H. In certain embodiments, R^{2d} is —CH₂CH(OH)(optionally substituted C₁-C₂₈ alkyl). In certain embodiments, R^{2d} is —CH₂CH(OH)(optionally substituted C₂-C₂₈ alkenyl). In certain embodiments, R^{2d} is —CH₂CH₂C(═O)O(optionally substituted C₁-C₂₈ alkyl). In certain embodiments, R^{2d} is —CH₂CH₂C(═O)NH(optionally substituted C₁-C₂₈ alkyl).

In certain embodiments, R^{2a} is —CH₂CH(OH)(CH₂)₉CH₃. In certain embodiments, R^{2a} is —CH₂CH(OH)(CH₂)₁₁CH₃. In certain embodiments, R^{2a} is —CH₂CH(OH)(CH₂)₁₃CH₃.

In certain embodiments, R^{2b} is —CH₂CH(OH)(CH₂)₉CH₃. In certain embodiments, R^{2b} is —CH₂CH(OH)(CH₂)₁₁CH₃. In certain embodiments, R^{2b} is —CH₂CH(OH)(CH₂)₁₃CH₃.

In certain embodiments, R^{2c} is —CH₂CH(OH)(CH₂)₉CH₃. In certain embodiments, R² is —CH₂CH(OH)(CH₂)₁₁CH₃. In certain embodiments, R^{2c} is —CH₂CH(OH)(CH₂)₁₃CH₃.

In certain embodiments, R^{2d} is —CH₂CH(OH)(CH₂)₉CH₃. In certain embodiments, R^{2d} is —CH₂CH(OH)(CH₂)₁₁CH₃. In certain embodiments, R^{2d} is —CH₂CH(OH)(CH₂)₁₃CH₃.

In certain embodiments, R³ is H. In certain embodiments, R³ is —CH₂CH(OH)(optionally substituted C₁-C₂₈ alkyl). In certain embodiments, R³ is —CH₂CH(OH)(optionally substituted C₂-C₂₈ alkenyl). In certain embodiments, R³ is —CH₂CH₂C(═O)O(optionally substituted C₁-C₂₈ alkyl). In certain embodiments, R³ is —CH₂CH₂C(═O)NH(optionally substituted C₁-C₂₈ alkyl).

In certain embodiments, R³ is —CH₂CH(OH)(CH₂)₉CH₃. In certain embodiments, R³ is —CH₂CH(OH)(CH₂)₁₁CH₃. In certain embodiments, R³ is —CH₂CH(OH)(CH₂)₁₃CH₃.

In certain embodiments, R⁴ is H.

In certain embodiments, R⁵ is H.

In certain embodiments, X is NH.

In certain embodiments, each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, and optionally substituted heteroalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′))(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound is

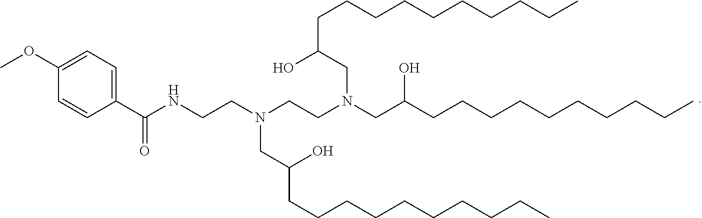 [SMILES: CCCCCCCCCCC(O)CN(CCNC(=O)c1ccc(OC)cc1)CCN(CC(O)CCCCCCCCCC)CC(O)CCCCCCCCCC]

In certain embodiments, the compound is

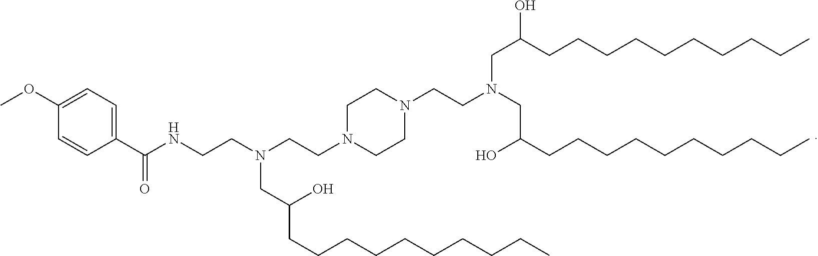 [SMILES: CCCCCCCCCCC(O)CN(CCNC(=O)c1ccc(OC)cc1)CCN1CCN(CCN(CC(O)CCCCCCCCCC)CC(O)CCCCCCCCCC)CC1]

In certain embodiments, the compound is

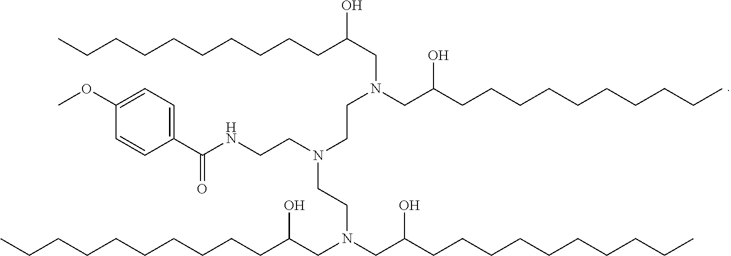 [SMILES: CCCCCCCCCCC(O)CN(CCN(CCNC(=O)c1ccc(OC)cc1)CCN(CC(O)CCCCCCCCCC)CC(O)CCCCCCCCCC)CC(O)CCCCCCCCCC]

In certain embodiments, the compound is

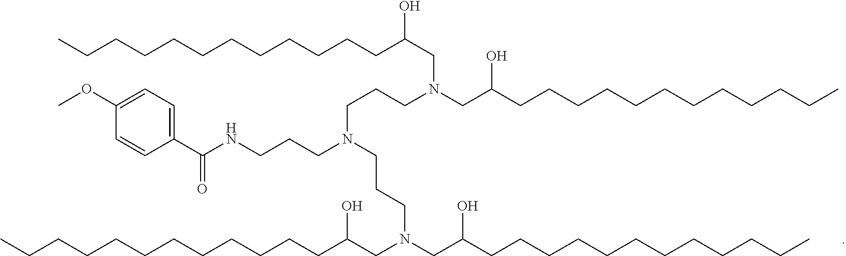 [SMILES: CCCCCCCCCCCCC(O)CN(CCCN(CCCNC(=O)c1ccc(OC)cc1)CCCN(CC(O)CCCCCCCCCCCC)CC(O)CCCCCCCCCCCC)CC(O)CCCCCCCCCCCC]

In certain embodiments, the compound is

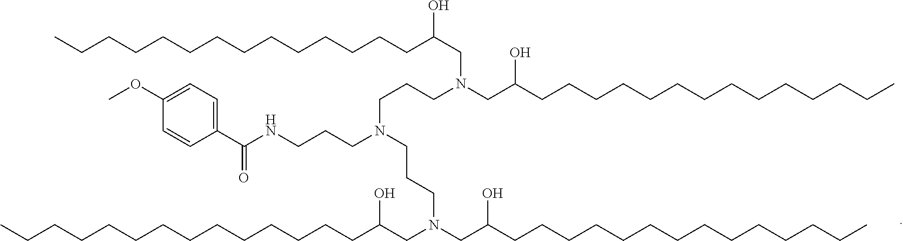 [SMILES: CCCCCCCCCCCCCCC(O)CN(CCCN(CCCNC(=O)c1ccc(OC)cc1)CCCN(CC(O)CCCCCCCCCCCCCC)CC(O)CCCCCCCCCCCCCC)CC(O)CCCCCCCCCCCCCC]

In certain embodiments, the compound is

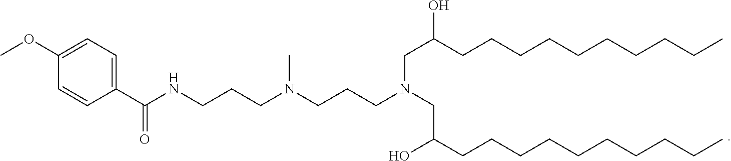 [SMILES: CCCCCCCCCCC(O)CN(CCCN(C)CCCNC(=O)c1ccc(OC)cc1)CC(O)CCCCCCCCCC]

In certain embodiments, the compound is

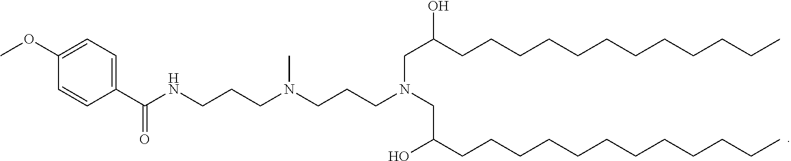 [SMILES: CCCCCCCCCCCCC(O)CN(CCCN(C)CCCNC(=O)c1ccc(OC)cc1)CC(O)CCCCCCCCCCCC]

In certain embodiments, the compound is

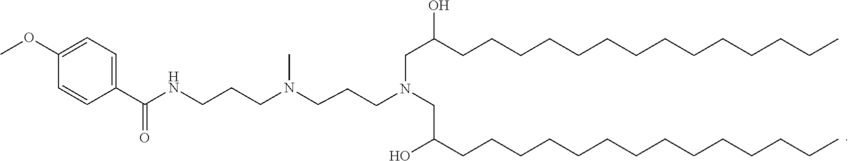 [SMILES: CCCCCCCCCCCCCCC(O)CN(CCCN(C)CCCNC(=O)c1ccc(OC)cc1)CC(O)CCCCCCCCCCCCCC]

In certain embodiments, the compound is

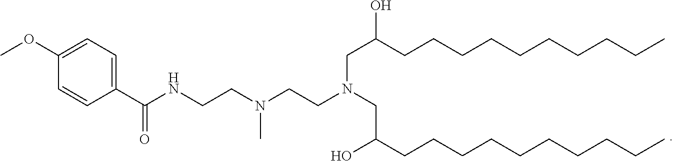 [SMILES: CCCCCCCCCCC(O)CN(CCN(C)CCNC(=O)c1ccc(OC)cc1)CC(O)CCCCCCCCCC]

In certain embodiments, the compound is

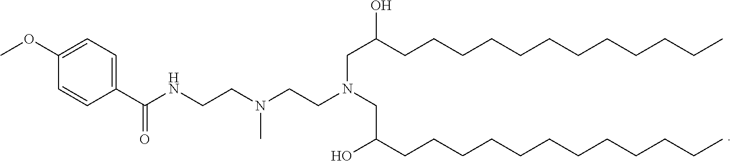 [SMILES: CCCCCCCCCCCCC(O)CN(CCN(C)CCNC(=O)c1ccc(OC)cc1)CC(O)CCCCCCCCCCCC]

In certain embodiments, the compound is

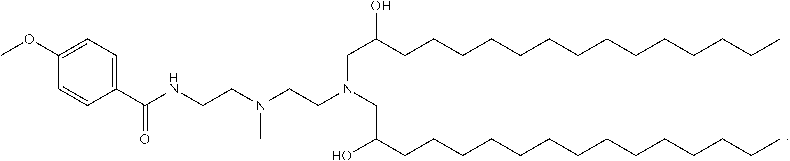 [SMILES: CCCCCCCCCCCCCCC(O)CN(CCN(C)CCNC(=O)c1ccc(OC)cc1)CC(O)CCCCCCCCCCCCCC]

In certain embodiments, the compound is

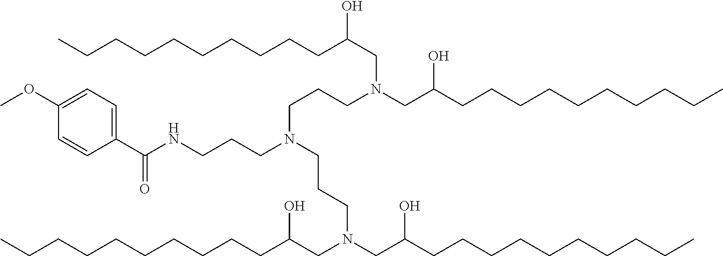 [SMILES: CCCCCCCCCCC(O)CN(CCCN(CCCNC(=O)c1ccc(OC)cc1)CCCN(CC(O)CCCCCCCCCC)CC(O)CCCCCCCCCC)CC(O)CCCCCCCCCC]

In certain embodiments, the compound is

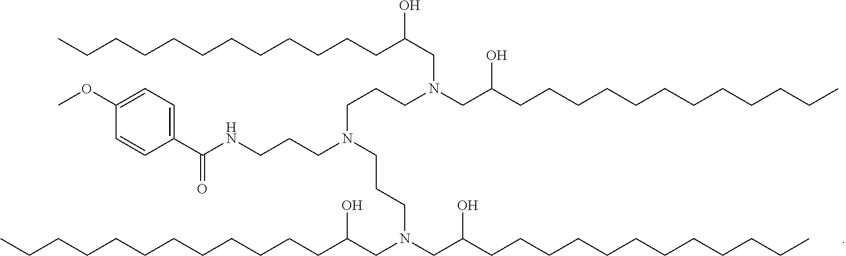 [SMILES: CCCCCCCCCCCCC(O)CN(CCCN(CCCNC(=O)c1ccc(OC)cc1)CCCN(CC(O)CCCCCCCCCCCC)CC(O)CCCCCCCCCCCC)CC(O)CCCCCCCCCCCC]

In certain embodiments, the compound is

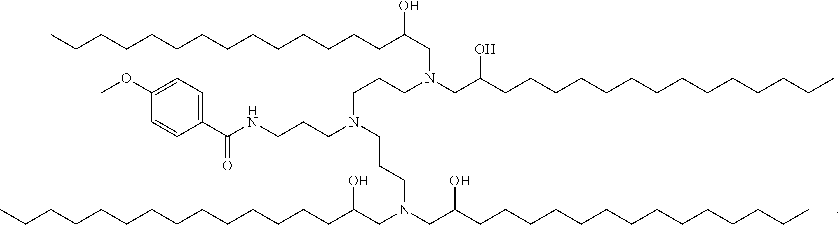 [SMILES: CCCCCCCCCCCCCCC(O)CN(CCCN(CCCNC(=O)c1ccc(OC)cc1)CCCN(CC(O)CCCCCCCCCCCCCC)CC(O)CCCCCCCCCCCCCC)CC(O)CCCCCCCCCCCCCC]

In certain embodiments the compound is

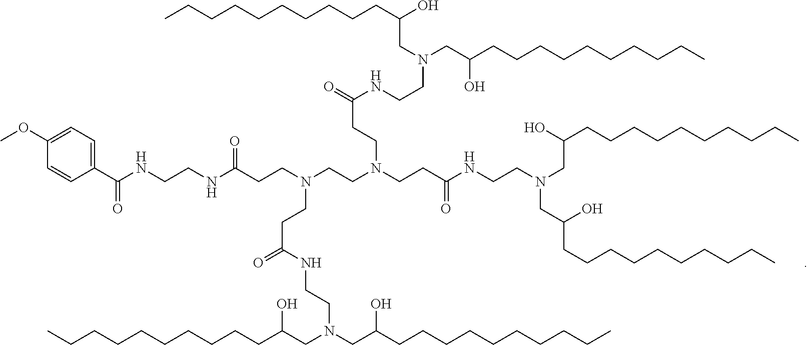 [SMILES: CCCCCCCCCCC(O)CN(CCNC(=O)CCN(CCC(=O)NCCNC(=O)c1ccc(OC)cc1)CCN(CCC(=O)NCCN(CC(O)CCCCCCCCCC)CC(O)CCCCCCCCCC)CCC(=O)NCCN(CC(O)CCCCCCCCCC)CC(O)CCCCCCCCCC)CC(O)CCCCCCCCCC]

In certain embodiments, the compound is

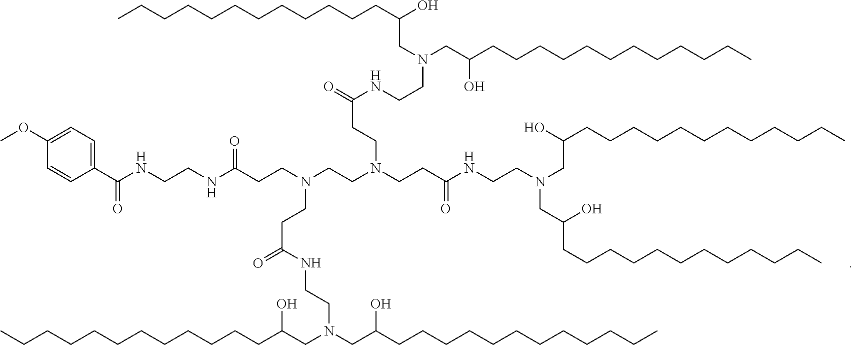 [SMILES: CCCCCCCCCCCCC(O)CN(CCNC(=O)CCN(CCC(=O)NCCNC(=O)c1ccc(OC)cc1)CCN(CCC(=O)NCCN(CC(O)CCCCCCCCCCCC)CC(O)CCCCCCCCCCCC)CCC(=O)NCCN(CC(O)CCCCCCCCCCCC)CC(O)CCCCCCCCCCCC)CC(O)CCCCCCCCCCCC]

In certain embodiments, the compound is

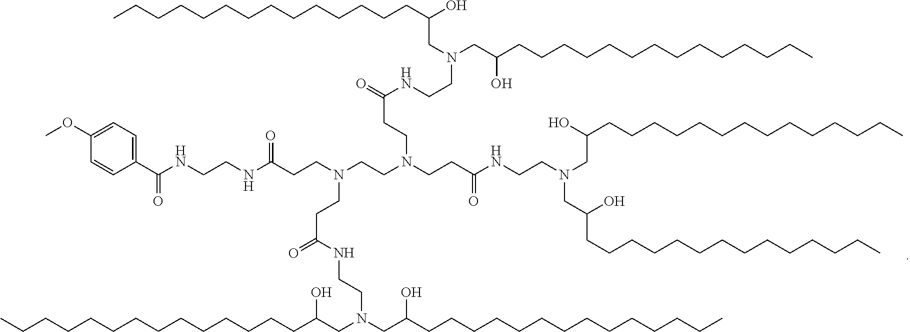 [SMILES: CCCCCCCCCCCCCCC(O)CN(CCNC(=O)CCN(CCC(=O)NCCNC(=O)c1ccc(OC)cc1)CCN(CCC(=O)NCCN(CC(O)CCCCCCCCCCCCCC)CC(O)CCCCCCCCCCCCCC)CCC(=O)NCCN(CC(O)CCCCCCCCCCCCCC)CC(O)CCCCCCCCCCCCCC)CC(O)CCCCCCCCCCCCCC]

In certain embodiments, the compound is

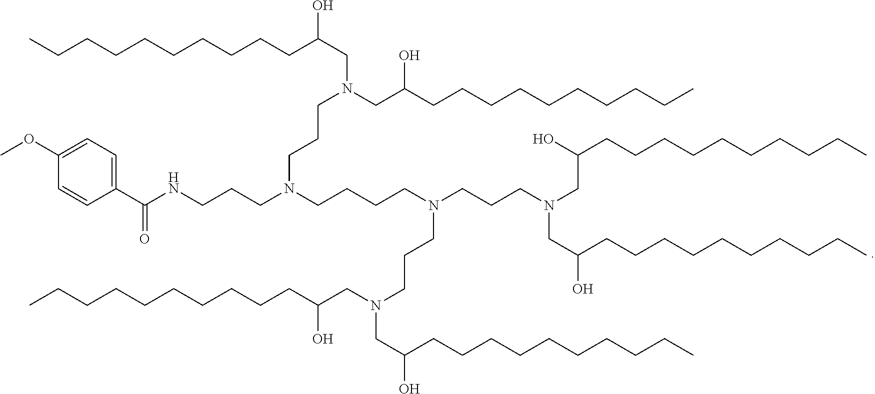 [SMILES: CCCCCCCCCCC(O)CN(CCCN(CCCCN(CCCN(CC(O)CCCCCCCCCC)CC(O)CCCCCCCCCC)CCCN(CC(O)CCCCCCCCCC)CC(O)CCCCCCCCCC)CCCNC(=O)c1ccc(OC)cc1)CC(O)CCCCCCCCCC]

In certain embodiments, the compound is

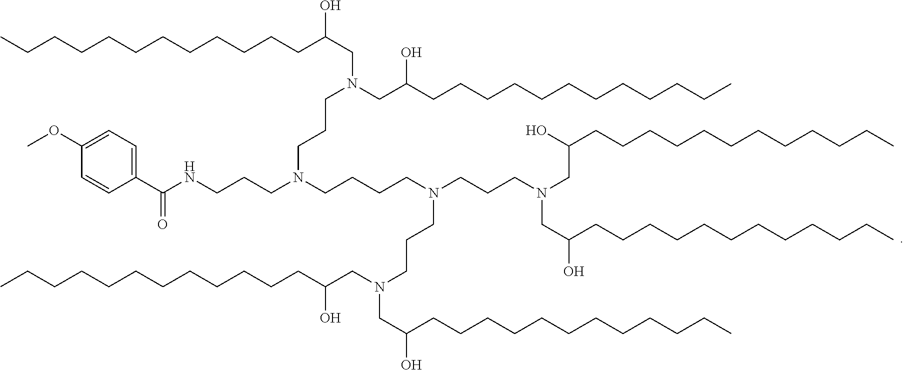 [SMILES: CCCCCCCCCCCCC(O)CN(CCCN(CCCCN(CCCN(CC(O)CCCCCCCCCCCC)CC(O)CCCCCCCCCCCC)CCCN(CC(O)CCCCCCCCCCCC)CC(O)CCCCCCCCCCCC)CCCNC(=O)c1ccc(OC)cc1)CC(O)CCCCCCCCCCCC]

In certain embodiments, the compound is

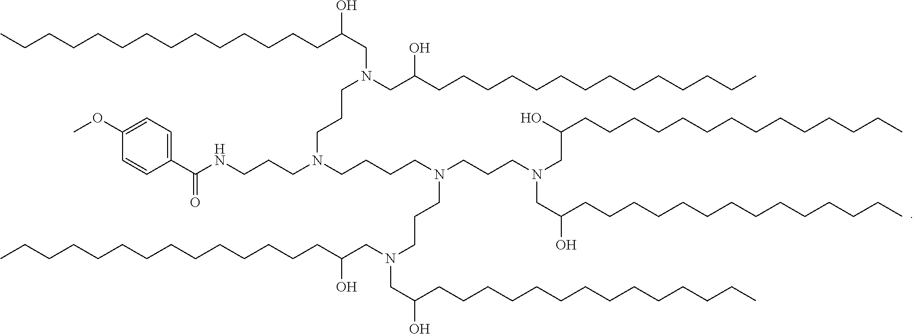 [SMILES: CCCCCCCCCCCCCCC(O)CN(CCCN(CCCCN(CCCN(CC(O)CCCCCCCCCCCCCC)CC(O)CCCCCCCCCCCCCC)CCCN(CC(O)CCCCCCCCCCCCCC)CC(O)CCCCCCCCCCCCCC)CCCNC(=O)c1ccc(OC)cc1)CC(O)CCCCCCCCCCCCCC]

In certain embodiments, the ionizable lipid compound of Formula (I) is:

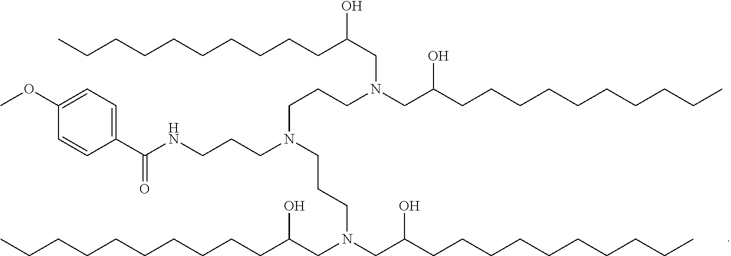 [SMILES: CCCCCCCCCCC(O)CN(CCCN(CCCNC(=O)c1ccc(OC)cc1)CCCN(CC(O)CCCCCCCCCC)CC(O)CCCCCCCCCC)CC(O)CCCCCCCCCC]

In certain embodiments, the compound comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the compound comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the compound comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the compound comprises about 50 mol % of the LNP. In certain embodiments, the compound comprises less than about 50 mol % of the LNP. In certain embodiments, the compound comprises more than about 50 mol % of the LNP.

In certain embodiments, the at least one helper lipid comprises distearoylphosphatidylcholine (DSPC).

In certain embodiments, the at least one helper lipid comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one helper lipid comprises less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one helper lipid comprises more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one helper lipid comprises about 10 mol % of the LNP. In certain embodiments, the at least one helper lipid comprises less than about 10 mol % of the LNP. In certain embodiments, the at least one helper lipid comprises more than about 10 mol % of the LNP.

In certain embodiments, the at least one cholesterol lipid comprises cholesterol.

In certain embodiments, the at least one cholesterol lipid comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the at least one cholesterol lipid comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the at least one cholesterol lipid comprises more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the cholesterol lipid comprises about 38.5 mol % of the LNP. In certain embodiments, the cholesterol lipid comprises less than about 38.5 mol % of the LNP. In certain embodiments, the cholesterol lipid comprises more than about 38.5 mol % of the LNP.

In certain embodiments, the at least one conjugated lipid comprises C14-PEG2000.

In certain embodiments, the at least one conjugated lipid comprises about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, or about 12.5 mol % of the LNP.

In certain embodiments, the at least one conjugated lipid comprises less than about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, or about 12.5 mol % of the LNP.

In certain embodiments, the at least one conjugated lipid comprises more than about 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, or about 12.5 mol % of the LNP.

In certain embodiments, the ratio of (a):(b):(c):(d) is about 50:10:38.5:1.5.

In certain embodiments, the ratio of (a):(b):(c):(d) is about 30:16:46.5:2.5.

In certain embodiments, the LNP selectively binds to at least one sigma receptor

In certain embodiments, the LNP selectively binds to at least one target cell of interest.

In certain embodiments, the target cell of interest comprises cell expressing at least one sigma receptor.

In certain embodiments, the LNP further comprises, or encapsulates, at least one additional agent.

In certain embodiments, the at least one additional agent is selected from the group consisting of a mRNA, a siRNA, a microRNA, a CRISPR-Cas9, a small molecule, a protein, an antibody, and any combination thereof.

In certain embodiments, the nucleic acid molecule is a DNA molecule or a RNA molecule. In certain embodiments, the nucleic acid molecule is selected from the group consisting of cDNA, mRNA, miRNA, siRNA, modified RNA, antagomir, antisense molecule, targeted nucleic acid, and any combination thereof.

In certain embodiments, the LNP further comprises mRNA.

In certain embodiments, the LNP has a ratio of lipids:mRNA of about 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and about 1:10.

### Cargo

In one aspect, the present invention relates to a composition comprising at least one compound or LNP of the present invention. In one aspect, the present invention relates to a composition comprising at least one compound or LNP of the present invention that selectively bind to at least one sigma receptor. Thus, in certain embodiments, the composition targets at least one cell expressing a sigma receptor. For example, in some embodiments, the composition targets at least one fibroblast, cancer cell, stromal cell, epithelial cell, or any combination thereof.

In one aspect, the invention is not limited to any particular cargo or otherwise agent for which the LNP is able to carry or transport. Rather, the invention includes can agent that can be carried by the LNP. For example, agents that can be carried by the LNP of the invention include, but are not limited to, diagnostic agents, detectable agents, and therapeutic agents.

In various embodiments, the composition comprises an in vitro transcribed (IVT) RNA molecule. For example, in certain embodiments, the composition of the invention comprises an IVT RNA molecule, which encodes an agent. In certain embodiments, the IVT RNA molecule of the present composition is a nucleoside-modified mRNA molecule.

In certain embodiments, the composition comprises at least one RNA molecule encoding a combination of at least two agents. In certain embodiments, the composition comprises a combination of two or more RNA molecules encoding a combination of two or more agents. In certain embodiments, the present invention provides a method for gene editing of a cell of interest of a subject. For example, the method can be used to provide one or more component of a gene editing system (e.g., a component of a CRISPR system) to a cell of interest of a subject. In some embodiments, the method comprises administering to the subject a composition comprising one or more ionizable LNP molecule formulated for targeted delivery comprising one or more nucleoside-modified RNA molecule for gene editing.

In certain embodiments, the method comprises administration of the composition to a subject. In certain embodiments, the method comprises administering a plurality of doses to the subject. In another embodiment, the method comprises administering a single dose of the composition, where the single dose is effective in delivery of the target therapeutic agent.

In one aspect, the composition of the present invention comprises one or more LNP formulated for targeted delivery of an agent to a cell of interest. Examples of such agents include, but are not limited to, a therapeutic agent, diagnostic agent, detectable agent, small molecule, peptide, polypeptide, amino acid molecule, nucleic acid molecule, drug, pro-drug, label, or any combination thereof.

For example, in some embodiments, the composition of the present invention comprises at least one therapeutic agent. In certain embodiments, the therapeutic agent is a hydrophobic therapeutic agent. In certain embodiments, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals, lipids, saccharides, metals, amino acids (and precursors), nucleic acids and precursors, contrast agents, diagnostic agents, dopamine receptor agonists, erectile dysfunction agents, fertility agents, gastrointestinal agents, hormones, immunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In certain embodiments, the therapeutic agent is one or more non-therapeutic moieties. In some embodiments, the nanoparticle comprises one or more therapeutic moieties, one or more non-therapeutic moieties, or any combination thereof. In certain embodiments, the therapeutic moiety targets cancer. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In certain embodiments, the therapeutic agent may be an anti-cancer agent. Any suitable anti-cancer agent may be used in the compositions and methods of the present disclosure. The selection of a suitable anti-cancer agent may depend upon, among other things, the type of cancer to be treated, ameliorated, and/or prevented and the nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but is not limited to, chemotherapeutic agents, antiproliferative agents, anti-tumor agents, checkpoint inhibitors, and anti-angiogenic agents. For example, In certain embodiments, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans-retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2) Small molecules, including topoisomerases such as razoxane, a topoisomerase II inhibitor with anti-angiogenic activity, can also be used.

Other anti-cancer agents that can be used in combination with the disclosed compounds include, but are not limited to acivicin, aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide, cytarabine; dacarbazine; dactinomycin; daunorubicin hydrochloride; decitabine; dexormaplatin; dezaguanine; dezaguanine mesylate; diaziquone; docetaxel, doxorubicin; doxorubicin hydrochloride; droloxifene; droloxifene citrate, dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide, floxuridine; fludarabine phosphate; fluorouracil; fluorocitabine; fosquidone; fostriecin sodium; gemcitabine; gemcitabine hydrochloride; hydroxyurea; idarubicin hydrochloride, ifosfamide; ilmofosine; interleukin II (including recombinant interleukin II, or rIL2), interferon alfa-2a; interferon alfa-2b; interferon alfa-n1, interferon alfa-n3; interferon beta-I a; interferon gamma-I b; iproplatin; irinotecan hydrochloride, lanreotide acetate; letrozole; leuprolide acetate; liarozole hydrochloride; lometrexol sodium; lomustine; losoxantrone hydrochloride; masoprocol, maytansine; mechlorethamine hydrochloride, megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole, nogalamycin; ormaplatin, oxisuran; paclitaxel; pegaspargase, peliomycin, pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine, thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard, uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin, zorubicin hydrochloride. Other anti-cancer drugs include, but are not limited to: 20-epi-1,25 dihydroxyvitamin D3; 5-ethynyluracil; abiraterone, aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma, antiestrogen; antineoplaston; antisense oligonucleotides; aphidicolin glycinate; apoptosis gene modulators; apoptosis regulators, apurinic acid; ara-CDP-DL-PTBA, arginine deaminase; asulacrine; atamestane; atrimustine; axinastatin 1; axinastatin 2; axinastatin 3; azasetron; azatoxin; azatyrosine, baccatin 111 derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breilate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide, cicaprost; cis-porphyrin; cladribine, clomifene analogues; clotrimazole; collismycin A; collismycin B; combretastatin A4; combretastatin analogue; conagenin; crambescidin 816; crisnatol; cryptophycin 8, cryptophycin A derivatives, curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel, docosanol; dolasetron; doxifluridine; droloxifene, dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine, fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides, maitansine; mannostatin A; marimastat; masoprocol; maspin; matrilysin inhibitors; matrix metalloproteinase inhibitors; menogaril; merbarone; meterelin; methioninase, metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim, monoclonal antibody, human chorionic gonadotrophin; monophosphoryl lipid A+myobacterium cell wall sk; mopidamol; multiple drug resistance gene inhibitor; multiple tumor suppressor 1-based therapy, mustard anticancer agent; mycaperoxide B; mycobacterial cell wall extract; myriaporone; N-acetyldinaline; N-substituted benzamides; nafarelin; nagrestip; naloxone+pentazocine; napavin, naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor, platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes, RII retinamide; rogletimide, rohitukine, romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor, stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium, tegafur; tellurapyrylium; telomerase inhibitors; temoporfin; temozolomide; teniposide; tetrachlorodecaoxide, tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin, tirapazamine; titanocene bichloride; topsentin; toremifene; totipotent stem cell factor; translation inhibitors; tretinoin; triacetyluridine; triciribine; trimetrexate; triptorelin, tropisetron; turosteride; tyrosine kinase inhibitors; tyrphostins, UBC inhibitors; ubenimex; urogenital sinus-derived growth inhibitory factor; urokinase receptor antagonists; vapreotide; variolin B; vector system, erythrocyte gene therapy; velaresol; veramine; verdins; verteportin; vinorelbine; vinxaltine; vitaxin; vorozole; zanoterone; zeniplatin; zilascorb; and zinostatin stimalamer. In certain embodiments, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the nanoparticle further comprises one or more gene components, such as siRNA or therapeutic DNA fragments. In some embodiments, the gene component is encapsulated in the nanoparticle. In some embodiments, the gene component is on the surface of the nanoparticle, for example, attached to or within the coating material.

In some embodiments, the nanoparticle further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of nanoparticle taking into consideration at least the type of imaging and/or therapy to be performed.

### Small Molecule

In various embodiments, the agent is a small molecule. In various embodiments, the agent is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means Biological means include purification from a biological source, recombinant synthesis, and in vitro translation systems, using methods well known in the art. In certain embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising a therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In certain embodiments, the therapeutic agent is a prodrug. In certain embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present invention can be used to treat a disease or disorder.

In certain embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Molecule

In other related aspects, the agent is a nucleic acid molecule. In various embodiments, the agent is an isolated nucleic acid. Thus, In certain embodiments, an isolated nucleic acid, including for example a DNA oligonucleotide and a RNA oligonucleotide can be incorporated in the composition of the invention. In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a cDNA, mRNA, siRNA, shRNA or miRNA molecule. In certain embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In certain embodiments, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In certain embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311, Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003), and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, CIT content at the termini, Tm and the nucleotide content of the 3′ overhang. See, for instance, Schwartz et al., 2003, Cell, 115:199-208 and Khvorova et al., 2003, Cell 115.209-216.

In one aspect, the invention includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using a the delivery vehicle of the invention. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In certain embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al, 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone, and/or the inclusion of nontraditional bases such as inosine, queuosine, and wybutosine and the like, as well as acetyl-methyl-, thio- and other modified forms of adenine, cytidine, guanine, thymine, and uridine.

In certain embodiments of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem 172.289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In certain embodiments of the invention, a ribozyme is used as a therapeutic agent to inhibit expression of a target protein. Ribozymes useful for inhibiting the expression of a target molecule may be designed by incorporating target sequences into the basic ribozyme structure, which are complementary, for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In certain embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In certain embodiments, the therapeutic agent comprises a gRNA or a nucleic acid molecule encoding a gRNA. In certain embodiments, the therapeutic agent comprises a Cas peptide or a nucleic acid molecule encoding a Cas peptide.

In certain embodiments, the agent comprises a miRNA or a mimic of a miRNA. In certain embodiments, the agent comprises a nucleic acid molecule that encodes a miRNA or mimic of a miRNA. miRNAs are small non-coding RNA molecules that are capable of causing post-transcriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In certain embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 100% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In certain embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In certain embodiments, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215: 403-410 (1990)].

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Polypeptide

In other related aspects, the agent is a polypeptide. In various embodiments, the agent is an isolated polypeptide. In other related aspects, the therapeutic agent includes an isolated polypeptide. For example, In certain embodiments, the polypeptide of the invention inhibits or activates a target directly by binding to the target thereby modulating the normal functional activity of the target. In certain embodiments, the polypeptide of the invention modulates the target by competing with endogenous proteins. In certain embodiments, the polypeptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

In one aspect, the invention includes an ionizable LNP molecule comprising or encapsulating one or more agent (e.g., a nucleic acid molecule) for targeted in vivo delivery of the encapsulated agent to a cell expressing a sigma receptor. In certain embodiments, the nucleic acid molecule is a mRNA molecule.

In some embodiments, the mRNA molecule comprises a nucleotide sequence that can alternatively comprise sequence variations with respect to the original nucleotide sequences, for example, substitutions, insertions and/or deletions of one or more nucleotides, with the condition that the resulting polynucleotide encodes a polypeptide according to the invention.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm (BLAST Manual, Altschul, S., et al., NCBI NLM NIH Bethesda, Md. 20894, Altschul, S., et al., J. Mol Biol. 215: 403-410 (1990)).

In certain embodiments, the composition comprises a plurality of constructs, each construct encoding one or more antigens. In certain embodiments, the composition comprises 1 or more, 2 or more, 5 or more, 10 or more, 15 or more, or 20 or more constructs. In certain embodiments, the composition comprises a first construct, comprising a nucleotide sequence encoding an antigen; and a second construct, comprising a nucleotide sequence encoding an adjuvant.

In certain embodiments, the construct comprises a plurality of nucleotide sequences encoding a plurality of antigens. In certain embodiments, the construct encodes 1 or more, 2 or more, 5 or more, 10 or more, 15 or more, or 20 or more antigens. In certain embodiments, the invention relates to a construct, comprising a nucleotide sequence encoding an adjuvant. For example, In certain embodiments, the construct comprises a first nucleotide sequence encoding an antigen and a second nucleotide sequence encoding an adjuvant.

In another particular embodiment, the construct is operatively bound to a translational control element. The construct can incorporate an operatively bound regulatory sequence for the expression of the nucleotide sequence of the invention, thus forming an expression cassette.

### Peptides

In certain embodiments, the agent is a peptide. Thus, in one aspect, a peptide can be incorporated into the LNP. Thus, In certain embodiments, the agent is a peptide. The peptide of the present invention may be made using chemical methods. For example, peptides can be synthesized by solid phase techniques (Roberge J Y et al (1995) Science 269. 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing. The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present invention includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Antibodies

In certain embodiments, the agent is an antibody. Thus, in various embodiments, the composition of the invention comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Chimeric Antigen Receptor (CAR) Agents

In certain embodiments, the agent comprises a nucleic acid sequence encoding a chimeric antigen receptor (CAR). In certain embodiments, the agent comprises an mRNA molecule encoding a CAR. In certain embodiments, the agent comprises a modified nucleoside mRNA molecule encoding a CAR.

In other embodiments, the LNP of the present invention is used to target a fibroblast and to engineer the fibroblast to express at least one CAR. In certain embodiments, the cell expressing CAR is a fibroblast cell (i.e., CAR fibroblast). Suitable CAR fibroblasts useful in the invention include those described in U.S. Patent Application Publication No. 2021/0171910, incorporated herein by reference in its entirety.

In certain embodiments, fibroblast cells express at least one CAR capable of endowing the fibroblast with the ability to trigger a T cell-mediated immune response. In certain embodiments, the CAR acts as a means of attaching or otherwise bridging fibroblasts to cancer cells. In another embodiment, the CAR acts as a means of triggering enhanced adhesion of the fibroblast to cancer cells. In certain embodiments, a CAR comprises an extracellular domain capable of binding an antigen, including a tumor or pathogen antigen.

Targets of antigen-specific targeting regions of CARs may be of any kind. In some embodiments, the antigen-specific targeting region of the CAR targets antigens specific for cancer, inflammatory disease, neuronal-disorders, diabetes, cardiovascular disease, infectious diseases or a combination thereof. Examples of antigens that may be targeted by the CARs include but are not limited to antigens expressed on B-cells, antigens expressed on carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases. The CARs of the disclosure may be capable of redirecting the effector function of the expressing-cells to the target antigen(s).

Antigens that may be targeted by the CARs of the disclosure include but are not limited to any one or more of 4-IBB, 707-AP, 5T4, adenocarcinoma antigen, alpha-fetoprotein, BAFF, B-lymphoma cell, C242 antigen, CA-125, carbonic anhydrase 9 (CA-IX), C-MET, ART-4, BAGE, b-catenin/m, bcr-abl, CAMEL, CAP-1, CCR4, CD 152, CD7, CD 19, CD20, CD200, CD22, CD221, CD23 (IgE receptor), CD28, CD30 (TNFRSF8), CD33, CD4, CD38, CD40, CD44 v6, CD44v7/8, CD51, CD52, CD56, CD74, CD80, CD93, CD123, CD171, CEA, CLPP, CNT0888, CTLA-4, carcinoembryonic antigen, EGP2, EGP40, DR5, ErbB2, ErbB3/4, EGFR, EpCAM, EPV-E6, CD3, CASP-8, CD109, CDK/4, CDC-27, Cyp-B, DAM-8, DAM-10, ELV-M2, ETV6, FAP, fibronectin extra domain-B, folate receptor 1, GAGE, GD2, GD3 ganglioside, glycoprotein 75, GPNMB, G250, Gp100, HAGE, HER2/neu, HGF, HMW-MAA, human scatter factor receptor kinase, hTERT, IGF-1 receptor, IGF-1, IgG1, —I-CAM, IL-13, IL-6, insulin-like growth factor I receptor, integrin a5α1, integrin avu3, Kappa or light chain, LAGE, Lewis Y, G250/CAIX, Glypican-3, MAGE, MCI-R, mesothelin, MORAb-009, MS4A1, MUC1, MUC16, mucin CanAg, N-glycolylneuraminic acid, NPC-1C, PDGF-R a, PDL192, phosphatidylserine, PSC1, PSMA, NKG2D ligands, RANKL, RON, ROR1, SAGE, SCH 900105, SDC1, SLAMF7, TAG-72, TEL/AML, tenascin C, TGF beta 2, TGF-β, TRAIL-R1, TRAIL-R2, tumor antigen CTAA16.88, VEGF-A, VEGFR-1, VEGFR2, vimentin, B7-H6, IL-13 receptor a2, IL-11 receptor Ra, 8H9, NCAM, Fetal AchR, iCE, MART-1, tyrosinase, WT-1, TEM-1, TEM-2, TEM-3, TEM-4, TEM-5, TEM-6, TEM-7, TEM-8, ROBO-4, and so forth. Other antigens specific for cancer will be apparent to those of skill in the art and may be used in connection with alternate embodiments of the invention.

Particular examples of target antigens include but are not limited to surface proteins found on cancer cells in a specific or amplified fashion (e.g. the IL-14 receptor, CD 19, CD20 and CD40 for B-cell lymphoma, the Lewis Y and CEA antigens for a variety of carcinomas, the Tag72 antigen for breast and colorectal cancer, EGF-R for lung cancer, folate binding protein and the HER-2 protein that is often amplified in human breast and ovarian carcinomas), or viral proteins (e.g. gp120 and gp41 envelope proteins of HIV, envelope proteins from the Hepatitis B and C viruses, the glycoprotein B and other envelope glycoproteins of human cytomegalovirus, the envelope proteins from oncoviruses such as Kaposi's sarcoma-associated Herpes virus). Other targets of the CARs of the disclosure include CD4, where the ligand is the HIV gp120 envelope glycoprotein, and other viral receptors, for example ICAM, which is the receptor for the human rhinovirus, and the related receptor molecule for poliovirus.

In some embodiments, the bispecific chimeric antigen receptors target and bind at least two different antigens. Examples of pairings of at least two antigens bound by the bispecific CARs of the disclosure include but are not limited to any combination with HER2, CD 19 and CD20, CD 19 and CD22, CD20 and —I-CAM, —I-CAM and GD2, EGFR and —I-CAM, EGFR and C-MET, EGFR and HER2, C-MET and HER2 and EGFR and ROR1. Other pairings of antigens specific for cancer will be apparent to those of skill in the art and may be used in connection with alternate embodiments of the disclosure. In yet other embodiments, the bispecific chimeric antigen receptor targets CD 19 and CD20.

Antigens specific for inflammatory diseases that may be targeted by the CARs of the disclosure include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-a, IFN-y, IgE, IgE Fc region, IL-1, IL-12, IL-23, IL-13, IL-17, IL-17A, IL-22, IL-4. IL-S, IL-5, IL-6, IL-6 receptor, integrin a4, integrin a407, Lamaglama, LFA-1 (CD11a), MEDI-528, myostatin, OX-40, rhuMAb (37, scleroscin, SOST, TGF beta 1, TNF-a or VEGF-A. Other antigens specific for inflammatory diseases will be apparent to those of skill in the art and may be used in connection with alternate embodiments of the invention.

Antigens specific for neuronal disorders that may be targeted by the CARs of the disclosure include but are not limited to any one or more of beta amyloid or MABT5102A. Other antigens specific for neuronal disorders will be apparent to those of skill in the art and may be used in connection with alternate embodiments of the invention.

Antigens specific for diabetes that may be targeted by the CARs of the disclosure include but are not limited to any one or more of L-43 or CD3. Other antigens specific for diabetes or other metabolic disorders will be apparent to those of skill in the art and may be used in connection with alternate embodiments of the invention.

Antigens specific for cardiovascular diseases which may be targeted by the CARs of the disclosure include but are not limited to any one or more of C5, cardiac myosin, CD41 (integrin alpha-lib), fibrin 11, beta chain, ITGB2 (CD 18) and sphingosine-1-phosphate. Other antigens specific for cardiovascular diseases will be apparent to those of skill in the art and may be used in connection with alternate embodiments of the disclosure.

Antigens specific for infectious diseases that may be targeted by the CARs of the disclosure include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-a. Other antigens specific for infectious diseases will be apparent to those of skill in the art and may be used in connection with alternate embodiments of the disclosure.

Additional targets of the CARs of the disclosure include antigens involved in B-cell associated diseases. Yet further targets of the CARs of the disclosure will be apparent to those of skill in the art and may be used in connection with alternate embodiments of the invention.

Other antigens specific for cancer will be apparent to those of skill in the art and may be used in connection with alternate embodiments of the invention.

In certain embodiments, the CAR comprises an antigen binding domain. In a particular non-limiting embodiment, the antigen-binding domain is an scFv specific for binding to a surface antigen of a target cell of interest (e.g., a cell expressing a sigma receptor, fibroblast, cancer cell, stromal cell, epithelial cell, etc.).

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol 173.2143-2150 (2004), Sadelain et al., Curr. Opin Immunol (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3s-chain, which is the primary transmitter of signals from endogenous T cell receptors (TCRs). “First generation” CARs can provide de novo antigen recognition and cause activation of both CD4+ and CD8+ T cells through their CD3s chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex.

“Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3s signaling domain. Preclinical studies have indicated that “Second Generation” CARs can improve the anti-tumor activity of cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3s activation domain.

“Fourth generation” CARs provide co-stimulation, for example, by CD28 or 4-1BB domains, and activation, for example, by a CD3s signaling domain in addition to a constitutive or inducible chemokine component.

“Fifth generation” CARs provide co-stimulation, for example, by CD28 or 4-BB domains, and activation, for example, by a CD3s signaling domain, a constitutive or inducible chemokine component, and an intracellular domain of a cytokine receptor, for example, IL-2Rₐ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above.

In some embodiments, the CAR-expressing fibroblasts comprise activity that may elicit an immune reaction and/or response in an individual. The activity may elicit the CAR-expressing fibroblasts to produce cytokines at a level higher than that in fibroblasts that do not express a CAR. In some embodiments, the CAR-expressing fibroblasts produce cytokines at a level higher than that of fibroblasts that do not express a CAR.

### Adjuvant

In certain embodiments, the agent is an adjuvant. Thus, in various embodiments, the composition comprises an adjuvant. In certain embodiments, the composition comprises a nucleic acid molecule encoding an adjuvant. In certain embodiments, the adjuvant-encoding nucleic acid molecule is IVT RNA. In certain embodiments, the adjuvant-encoding nucleic acid molecule is nucleoside-modified mRNA.

Exemplary adjuvants include, but is not limited to, alpha-interferon, gamma-interferon, platelet derived growth factor (PDGF), TNF-α, TNF-β, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-Ia, MIP-Ip, IL-8, RANTES, L-selectin, P-selectin, E-selectin, CD34, GlyCAM-1, MadCAM-1, LFA-1, VLA-1, Mac-I, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

### Nucleoside-Modified RNA

In certain embodiments, the agent is a nucleoside-modified RNA. Thus, in one aspect, the composition comprises a nucleoside-modified RNA. Thus, In certain embodiments, the agent is a nucleoside-modified RNA In certain embodiments, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present invention is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Karik6 et al., 2008, Mol Ther 16:1833-1840; Karik6 et al., 2012, Mol Ther 20:948-953). The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karik6 et al., 2008, Mol Ther 16:1833-1840; Anderson et al., 2010, Nucleic Acids Res 38:5884-5892; Anderson et al., 2011, Nucleic Acids Research 39:9329-9338; Karik6 et al., 2011, Nucleic Acids Research 39:e142; Karik6 et al., 2012, Mol Ther 20.948-953; Karik6 et al., 2005, Immunity 23:165-175).

It has been demonstrated that the presence of modified nucleosides, including pseudouridines in RNA suppress their innate immunogenicity (Karik6 et al., 2005, Immunity 23:165-175). Further, protein-encoding, in vitro-transcribed RNA containing pseudouridine can be translated more efficiently than RNA containing no or other modified nucleosides (Karik et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Karik6 et al., 2011, Nucleic Acids Research 39:e142). Administering HPLC-purified, pseudourine-containing RNA coding for erythropoietin into mice and macaques resulted in a significant increase of serum EPO levels (Karik6 et al., 2012, Mol Ther 20:948-953), thus confirming that pseudouridine-containing mRNA is suitable for in vivo protein therapy.

The present invention encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside.

In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In certain embodiments, the nucleoside-modified RNA of the invention is IVT RNA. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In certain embodiments, the modified nucleoside is m¹acp³P (1-methyl-3-(3-amino-3-carboxypropyl) pseudouridine. In another embodiment, the modified nucleoside is m¹′P (1-25 methylpseudouridine). In another embodiment, the modified nucleoside is ′Pm (2′-O-methylpseudouridine. In another embodiment, the modified nucleoside is m⁵D (5-methyldihydrouridine). In another embodiment, the modified nucleoside is m³′P (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the modified nucleoside of the present invention is m⁵C-(5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is ′P (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m⁶A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl)adenosine); g⁶A (N-glycinylcarbamoyladenosine), t⁶A (N⁸-threonylcarbamoyladenosine); ms²t⁶A (2-methylthio-N-threonyl carbamoyladenosine); m⁶t⁶A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac⁴C (N⁴-acetylcytidine); f⁵C (5-formylcytidine); m⁵Cm (5,2′-O-dimethylcytidine); ac⁴Cm (Na-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)), yW (wybutosine), o₂yW (peroxywybutosine); OHyW (hydroxywybutosine); OHyW* (undermodified hydroxywybutosine), imG (wyosine); mimG (methylwyosine); Q (queuosine), oQ (epoxyqueuosine); galQ (galactosyl-queuosine), manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine), ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s2U (5-carboxymethylaminomethyl-2-thiouridine); m⁶2A (N⁶,N -dimethyladenosine); Im (2′-O-methylinosine); m⁴C (N′-methylcytidine); m⁴Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dinethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); Tm⁵U (5-taurinomethyluridine); Tm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present invention comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present invention are modified (e.g. either by the presence of pseudouridine or a modified nucleoside base) In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60% In another embodiment, the fraction is 700%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4% In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20% In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10% In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present invention is translated in the cell more efficiently than an unmodified RNA molecule with the same sequence. In another embodiment, the nucleoside-modified RNA exhibits enhanced ability to be translated by a target cell. In another embodiment, translation is enhanced by a factor of 2-fold relative to its unmodified counterpart. In another embodiment, translation is enhanced by a 3-fold factor. In another embodiment, translation is enhanced by a 5-fold factor In another embodiment, translation is enhanced by a 7-fold factor. In another embodiment, translation is enhanced by a 10-fold factor In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified antigen-encoding RNA of the present invention induces significantly more adaptive immune response than an unmodified in vitro-synthesized RNA molecule with the same sequence. In another embodiment, the modified RNA molecule exhibits an adaptive immune response that is 2-fold greater than its unmodified counterpart. In another embodiment, the adaptive immune response is increased by a 3-fold factor. In another embodiment the adaptive immune response is increased by a 5-fold factor. In another embodiment, the adaptive immune response is increased by a 7-fold factor. In another embodiment, the adaptive immune response is increased by a 10-fold factor. In another embodiment, the adaptive immune response is increased by a 15-fold factor. In another embodiment the adaptive immune response is increased by a 20-fold factor. In another embodiment, the adaptive immune response is increased by a 50-fold factor. In another embodiment, the adaptive immune response is increased by a 100-fold factor. In another embodiment, the adaptive immune response is increased by a 200-fold factor. In another embodiment, the adaptive immune response is increased by a 500-fold factor. In another embodiment, the adaptive immune response is increased by a 1000-fold factor. In another embodiment, the adaptive immune response is increased by a 2000-fold factor. In another embodiment, the adaptive immune response is increased by another fold difference.

In another embodiment, “induces significantly more adaptive immune response” refers to a detectable increase in an adaptive immune response. In another embodiment, the term refers to a fold increase in the adaptive immune response (e.g., 1 of the fold increases enumerated above) In another embodiment, the term refers to an increase such that the nucleoside-modified RNA can be administered at a lower dose or frequency than an unmodified RNA molecule with the same species while still inducing an effective adaptive immune response. In another embodiment, the increase is such that the nucleoside-modified RNA can be administered using a single dose to induce an effective adaptive immune response.

In another embodiment, the nucleoside-modified RNA of the present invention exhibits significantly less innate immunogenicity than an unmodified in vitro-synthesized RNA molecule with the same sequence. In another embodiment, the modified RNA molecule exhibits an innate immune response that is 2-fold less than its unmodified counterpart. In another embodiment, innate immunogenicity is reduced by a 3-fold factor. In another embodiment, innate immunogenicity is reduced by a 5-fold factor. In another embodiment, innate immunogenicity is reduced by a 7-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the recombinant protein. In another embodiment, the decrease is such that the nucleoside-modified RNA can be repeatedly administered without eliciting an innate immune response sufficient to eliminate detectable production of the recombinant protein.

### Combinations

In certain embodiments, the composition of the present invention comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In certain embodiments, the composition comprises a 1:1 ratio of two individual agents. However, the combination is not limited to any particular ratio. Rather any ratio that is shown to be effective is encompassed.

### Methods

The present invention provides methods of delivering an agent to a cell of interest (e.g, a cell expressing a sigma receptor) of a target subject. Exemplary cells expressing a sigma receptor that can be targeted using the LNP compositions of the invention include, but are not limited to, fibroblasts, cancer cells, stromal cells, and epithelial cells.

In some embodiments, the agent is a diagnostic agent to detect at least one marker associated with a disease or disorder. In some embodiments, the agent is a therapeutic agent for the treatment, amelioration, and/or prevention of a disease or disorder. Therefore, in some embodiments, the invention provides methods for diagnosing, treating, and/or preventing a disease or disorder comprising administering an effective amount of the LNP composition comprising one or more diagnostic or therapeutic agents, one or more adjuvants, or a combination thereof.

For example, In certain embodiments, the disease or disorder is fibrosis, a fibrotic condition, disease or disorder associated with fibrosis, fibrotic disease or disorder, or any combination thereof. In some embodiments, fibrosis may be triggered by pathological conditions, e.g. conditions, infections or disease states that lead to production of pro-fibrotic factors such as TGF-β1. In some embodiments, fibrosis may be caused by physical injury/stimuli, chemical injury/stimuli or environmental injury/stimuli. Physical injury/stimuli may occur during surgery, e.g., iatrogenic causes. Chemical injury/stimuli may include drug induced fibrosis, e.g. following chronic administration of drugs such as bleomycin, cyclophosphamide, amiodarone, procainamide, penicillamine, gold and nitrofurantoin (e.g., Daba et al., Saudi Med J 2004 June; 25(6): 700-6) Environmental injury/stimuli may include exposure to asbestos fibers or silica.

In some embodiments, fibrosis may involve an organ of the gastrointestinal system, e.g. of the liver, small intestine, large intestine, or pancreas. In some embodiments, fibrosis may involve an organ of the respiratory system, e.g., the lungs. In embodiments, fibrosis may involve an organ of the cardiovascular system, e.g., of the heart or blood vessels. In some embodiments, fibrosis may involve the skin. In some embodiments, fibrosis may involve an organ of the nervous system, e.g., the brain. In some embodiments, fibrosis may involve an organ of the urinary system, e.g., the kidneys. In some embodiments, fibrosis may involve an organ of the musculoskeletal system, e.g., muscle tissue.

Fibrosis can occur in many tissues of the body. For example, fibrosis can occur in the liver (e.g., cirrhosis), lungs, kidney, heart, blood vessels, eye, skin, pancreas, intestine, brain, and bone marrow. Fibrosis may also occur in multiple organs at once. Examples of fibrotic diseases or disorders that can be treated, ameliorated, and/or prevented using the LNP compositions of the invention include, but are not limited to, pulmonary fibrosis, cystic fibrosis, fibrothorax, idiopathic pulmonary fibrosis, bridging fibrosis, cirrhosis, scleroderma, glial scar, liver fibrosis, myocardial fibrosis, interstitial fibrosis, replacement fibrosis, subepithelial fibrosis, arterial stiffness, arthrofibrosis, chronic kidney disease, Hermansky-Pudlak syndrome, Crohn's disease, dupuytren's contracture, keloid, mediastinal fibrosis, myelofibrosis, sarcoidosis, peyronie's disease, nephrogenic systemic fibrosis, progressive massive fibrosis, retroperitoneal fibrosis, scleroderma, systemic sclerosis, adhesive capsulitis, arthritis; fibrotic pre-neoplastic and fibrotic neoplastic disease; and fibrosis induced by chemical or environmental insult (e.g., cancer chemotherapy, pesticides, radiation/cancer radiotherapy), and any combination thereof.

In some embodiments, the invention relates to methods of treating, ameliorating, and/or preventing cardiovascular conditions and diseases or disorders associated therewith in subjects in need thereof, the method comprising administering the LNP composition of the invention. Exemplary cardiovascular conditions that can be treated, ameliorated, and/or prevented using the LNP compositions and methods of the invention include, but are not limited to, hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, and cerebral infarcts.

In some embodiments, the invention relates to methods of treating, ameliorating, and/or preventing neurological conditions and diseases or disorders associated therewith in subjects in need thereof, the method comprising administering the LNP composition of the invention. Exemplary neurological conditions that can be treated, ameliorated, and/or prevented using the LNP compositions and methods of the invention include, but are not limited to, amyotrophic lateral sclerosis/frontotemporal dementia, gliosis, Huntington's diseases (HD) and Alzheimer's disease (AD).

In some embodiments, the invention relates to methods of treating, ameliorating, and/or preventing muscular dystrophy such as Duchenne muscular dystrophy (DMD) or Becker's muscular dystrophy (BMD) and diseases or disorders associated therewith in subjects in need thereof, the method comprising administering the LNP composition of the invention.

In some embodiments, the invention relates to methods of treating, ameliorating, and/or preventing gastrointestinal conditions such as Chron's disease, microscopic colitis and primary sclerosing cholangitis (PSC) and diseases or disorders associated therewith in subjects in need thereof, the method comprising administering the LNP composition of the invention.

In some embodiments, the invention relates to methods of treating, ameliorating, and/or preventing skin conditions and diseases or disorders associated therewith in subjects in need thereof, the method comprising administering the LNP composition of the invention. Exemplary skin conditions that can be treated, ameliorated, and/or prevented using the LNP compositions and methods of the invention include, but are not limited to, scleroderma, nephrogenic systemic fibrosis and cutis keloid; arthrofibrosis; Dupuytren's contracture; mediastinal fibrosis; retroperitoneal fibrosis; myelofibrosis; Peyronie's disease and adhesive capsulitis.

In some embodiments, the invention relates to methods of treating, ameliorating, and/or preventing kidney diseases or disorders in subjects in need thereof, the method comprising administering the LNP composition of the invention Exemplary kidney diseases or disorders that can be treated, ameliorated, and/or prevented using the LNP compositions and methods of the invention include, but are not limited to, renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS) and chronic graft versus host disease.

In some embodiments, the invention relates to methods of treating, ameliorating, and/or preventing diseases or disorders associated with the eye in subjects in need thereof, the method comprising administering the LNP composition of the invention. Exemplary eye disease or disorders that can be treated, ameliorated, and/or prevented using the LNP compositions and methods of the invention include, but are not limited to, Grave's ophthalmopathy, epiretinal fibrosis, retinal fibrosis, subretinal fibrosis (e.g., associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, and subconjunctival fibrosis.

In some embodiments, the invention relates to methods of treating, ameliorating, and/or preventing cancer and diseases or disorders associated therewith in subjects in need thereof, the method comprising administering the LNP composition of the invention.

In some embodiments, the present invention provides a method for inducing an immune response in subjects in need thereof, the method comprising administering the LNP composition of the invention. For example, In certain embodiments, the method for inducing an immune response in subjects in need thereof is a cancer immunotherapy comprising administering the LNP-modified CAR fibroblast to the subject to induce an immune response against cancer.

Exemplary cancers that can be treated, ameliorated, and/or prevented using the LNP compositions and methods of the invention include, but are not limited to, acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, Hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocvtoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer, transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In various embodiments, the disease or disorder is a disease or disorder associated with the level or activity of at least one sigma receptor. For example, In certain embodiments, the disease or disorder associated with the level or activity of at least one sigma receptor is cancer. In certain embodiments, the method comprises administering a LNP composition of the invention comprising one or more nucleic acid molecules for treatment or prevention of a disease or disorder (e.g., a disease or disorder associated with fibrosis, a neurological disease or disorder, a skin condition, or a cancer or disease or disorder associated therewith). In certain embodiments, the one or more nucleic acid molecules encode a therapeutic agent for the treatment of the disease or disorder (e.g., a disease or disorder associated with fibrosis, a neurological disease or disorder, a skin condition, or a cancer or disease or disorder associated therewith).

In certain embodiments, the compositions of the invention can be administered in combination with one or more additional therapeutic agent, an adjuvant, or a combination thereof. For example, In certain embodiments, the method comprises administering an LNP composition comprising a nucleic acid molecule encoding one or more agent for targeted administration to a cell of interest (e.g., a cell expressing a sigma receptor, fibroblast, cancer cell, stromal cell, epithelial cell, etc.) and a second LNP comprising a nucleic acid molecule encoding one or more adjuvants. In certain embodiments, the method comprises administering a single LNP composition comprising a nucleic acid molecule encoding one or more agent for targeted administration to a cell of interest (e.g, a cell expressing a sigma receptor, fibroblast, cancer cell, stromal cell, epithelial cell, etc.) and a nucleic acid molecule encoding one or more adjuvants.

In certain embodiments, the method comprises administering to subject a plurality of LNPs of the invention comprising nucleoside-modified nucleic acid molecules encoding a plurality of agents to a cell of interest (e.g., a cell expressing a sigma receptor, fibroblast, cancer cell, stromal cell, epithelial cell, etc.), adjuvants, or a combination thereof.

In certain embodiments, the method comprises administering the LNP of the invention comprising nucleoside-modified RNA, which provides stable expression of a nucleic acid encoded agent (e.g., a therapeutic agent encoded by a nucleoside modified mRNA molecule) described herein to a cell of interest (e.g., a cell expressing a sigma receptor, fibroblast, cancer cell, stromal cell, epithelial cell, etc.).

Administration of the compositions of the invention in a method of treatment can be achieved in a number of different ways, using methods known in the art. In certain embodiments, the method of the invention comprises systemic administration of the subject, including for example enteral or parenteral administration. In certain embodiments, the method comprises intradermal delivery of the composition. In another embodiment, the method comprises intravenous delivery of the composition. In some embodiments, the method comprises intramuscular delivery of the composition. In certain embodiments, the method comprises subcutaneous delivery of the composition. In certain embodiments, the method comprises inhalation of the composition. In certain embodiments, the method comprises intranasal delivery of the composition.

It will be appreciated that the composition of the invention may be administered to a subject either alone, or in conjunction with another agent.

The therapeutic and prophylactic methods of the invention thus encompass the use of pharmaceutical compositions comprising at least one LNP of the invention comprising an agent (e.g., an mRNA molecule) described herein, to practice the methods of the invention. The pharmaceutical compositions useful for practicing the invention may be administered to deliver a dose of from ng/kg/day and 100 mg/kg/day. In certain embodiments, the invention envisions administration of a dose which results in a concentration of the compound of the present invention from 10 nM and 10 μM in a mammal.

Typically, dosages which may be administered in a method of the invention to a mammal, preferably a human, range in amount from 0.01 μg to about 50 mg per kilogram of body weight of the mammal, while the precise dosage administered will vary depending upon any number of factors, including but not limited to, the type of mammal and type of disease state being treated, ameliorated, and/or prevented, the age of the mammal and the route of administration. Preferably, the dosage of the compound will vary from about 0.1 μg to about 10 mg per kilogram of body weight of the mammal. More preferably, the dosage will vary from about 1 μg to about 1 mg per kilogram of body weight of the mammal.

The composition may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, ameliorated, and/or prevented, the type and age of the mammal, etc.

In certain embodiments, administration of a composition of the present invention may be performed by single administration or boosted by multiple administrations.

In certain embodiments, the invention includes a method comprising administering a combination of LNP compositions described herein. In certain embodiments, the combination has an additive effect, wherein the overall effect of the administering the combination is approximately equal to the sum of the effects of administering each LNP composition. In other embodiments, the combination has a synergistic effect, wherein the overall effect of administering the combination is greater than the sum of the effects of administering each LNP composition.

In some aspects of the invention, the method provides for delivery of compositions for gene editing or genetic manipulation to a target cell (e.g., a cell expressing a sigma receptor, fibroblast, cancer cell, stromal cell, epithelial cell, etc.) of a subject to treat or prevent a disease or disorder (e.g., a disease or disorder associated with fibrosis, a neurological disease or disorder, a skin condition, a cancer or disease or disorder associated therewith, or a disease or disorder associated with the level or activity of at least one sigma receptor).

### Therapy

In one aspect, the therapeutic compounds or compositions of the invention may be administered prophylactically (i.e., to prevent disease or disorder, such as a disease or disorder associated with fibrosis, a neurological disease or disorder, a skin condition, a cancer or disease or disorder associated therewith, or a disease or disorder associated with the level or activity of at least one sigma receptor) or therapeutically (i.e., to treat disease or disorder, such as a disease or disorder associated with fibrosis, a neurological disease or disorder, a skin condition, a cancer or disease or disorder associated therewith, or a disease or disorder associated with the level or activity of at least one sigma receptor) to subjects suffering from or at risk of (or susceptible to) developing the disease or disorder (e.g., a disease or disorder associated with fibrosis, a neurological disease or disorder, a skin condition, a cancer or disease or disorder associated therewith, or a disease or disorder associated with the level or activity of at least one sigma receptor). Such subjects may be identified using standard clinical methods.

In the context of the present invention, prophylactic administration occurs prior to the manifestation of overt clinical symptoms of disease or disorder (e.g., a disease or disorder associated with fibrosis, a neurological disease or disorder, a skin condition, a cancer or disease or disorder associated therewith, or a disease or disorder associated with the level or activity of at least one sigma receptor), such that the disease or disorder is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The composition of the invention can be useful in combination with therapeutic, anti-cancer, and/or radiotherapeutic agents. Thus, the present disclosure provides a combination of the present LNP with therapeutic, anti-cancer, and/or radiotherapeutic agents for simultaneous, separate, or sequential administration. The composition of the invention and the other anticancer agent can act additively or synergistically.

The therapeutic agent, anti-cancer agent, and/or radiation therapy can be administered according to therapeutic protocols well known in the art. It will be apparent to those skilled in the art that the administration of the therapeutic agent, anti-cancer agent, and/or radiation therapy can be varied depending on the disease being treated, ameliorated, and/or prevented and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt. As used herein, “additional ingredients” include, but are not limited to, one or more of the following excipients, surface active agents; dispersing agents; inert diluents, granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents, antioxidants; antibiotics, antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### EXAMPLES

Various embodiments of the present application can be better understood by reference to the following Examples which are offered by way of illustration. The scope of the present application is not limited to the Examples given herein.

### Materials and Methods

### Materials

Core 200 was customized from Enamine (Monmouth Junction, NJ) and other polyamine cores were purchased from Sigma Aldrich, Tokyo Chemical Industry (TCI) and Alfa Aesar. Epoxydodecane (C12), epoxytetradecane (C14), epoxyhexadecane (C16), 4-methoxybenzoic acid, N,N′-Dicyclohexylcarbodiimide (DCC), N-Hydroxysuccinimide (NHS), HSP47 siRNA pool (NM_001111043, NM_001111044 and NM_009825) and Cy5-siRNA were purchased from Sigma Aldrich. GFP siRNA (cat. P-002048-01-50) and DharmaFECT transfection reagents were bought from Horizon Discovery Ltd. 1% agarose gels with SYBR™ Safe (#A42100) were purchased from ThermoFisher. Recombinant mouse TGF-β1 (cat. 7666-MB) was obtained from R&D. DSPC (#850365), cholesterol (#700100) and C14-PEG2000 (#880150) was bought from Avanti Polar Lipids. DLin-MC3-DMA was purchased from MedChem Express (Monmouth Junction, NJ). Luciferase mRNA was produced as previously described⁴⁶.

### Synthesis of Anisoyl-NHS Ester

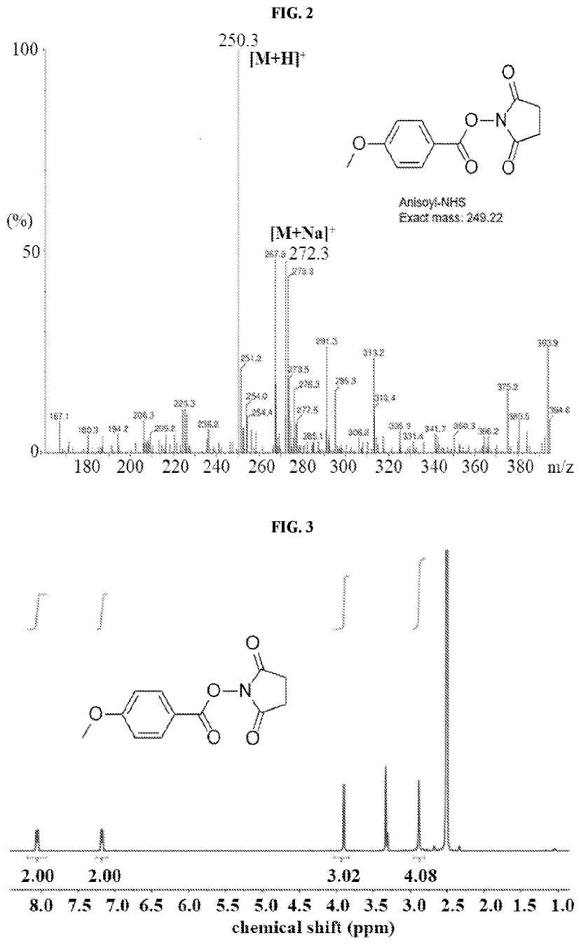

4-methoxybenzoic acid (960 mg, 6.4 mmol) and NHS (800 mg, 7 mmol) were dissolved in 36 mL DCM and stirred at 0° C. DCC (1440 mg, 7 mmol) in 40 mL DCM was added drop-wise for 30 min. The resulting mixture was further stirred at 0° C. for 1 h and then placed in a refrigerator at 0° C. overnight. The precipitated solid was removed by filtration, and the filtrate was dried to give a crude product. Recrystallization of the crude solid from 2-propanol afforded 1.2 g of Anisoyl-NHS ester (yield 75%). It was characterized by mass spectrometry (FIG. 2) and nuclear magnetic resonance spectroscopy (FIG. 3).

### General Method for the Synthesis of Anisamide-Tethered Lipidoids

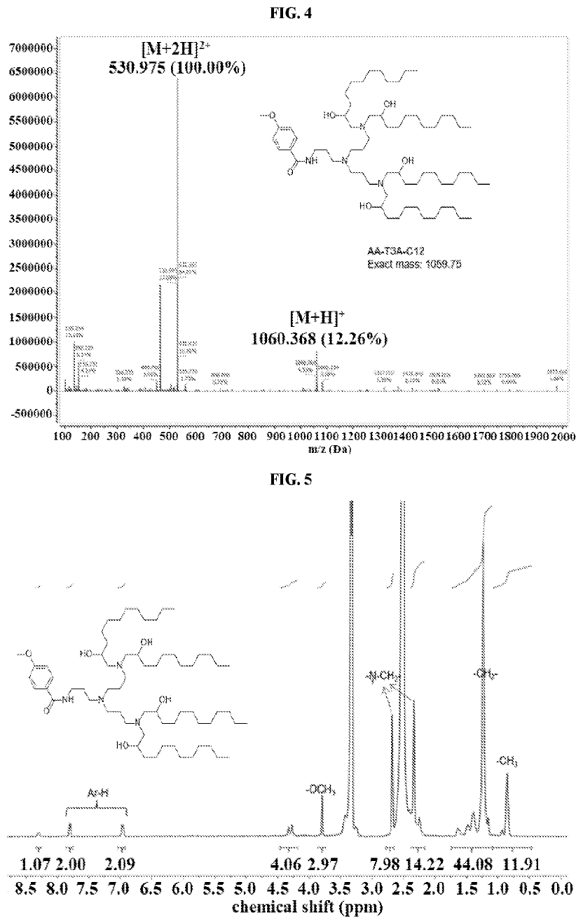

AA-lipidoids were synthesized using a one-pot, two-step method. First, polyamine (1 equiv.) and anisoyl-NHS ester (1 equiv.) were combined in EtOH, and TEA (1.2 equiv.) was then added. The resulting mixture was mildly heated (30-50° C.) for 2 h, in which, anisoyl-NHS ester was slowly dissolved and reacted with polyamine. Afterwards, excessive alkyl epoxide (e.g., 4.8 equiv. for T3A core) was added and the mixture was heated at 80° C. for 2 days. Crude products were used for initial in vitro screening. The top-performing AA-lipidoid, AA-T3A-C12 was purified by a CombiFlash Nextgen 300+ chromatography system (Teledyne ISCO) with gradient elution from CH₂Cl₂ to 75:22:3 CH₂Cl₂/MeOH/NH₄OH (aq). The target fraction was identified by mass spectrometry (FIG. 4) and nuclear magnetic resonance spectroscopy (FIG. 5). Exemplary AA-functionalized amine cores and exemplary AA-lipidoids are indicated in Tables 1-2.

TABLE 1
Exemplary AA-functionalized amine cores

| Name | Structure |
|---|---|
| AA-114 | 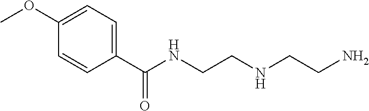 [SMILES: COc1ccc(C(=O)NCCNCCN)cc1] |
| | N-(2-((2-aminoethyl)amino)ethyl)-4-methoxybenzamide |
| AA-200 | 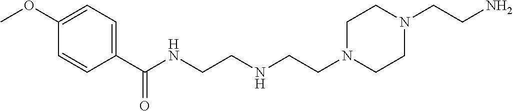 [SMILES: COc1ccc(C(=O)NCCNCCN2CCN(CCN)CC2)cc1] |
| | N-(2-((2-(4-(2-aminoethyl)piperazin-1-yl)ethyl)amino)ethyl)-4- |
| | methoxybenzamide |
| AA-110 | 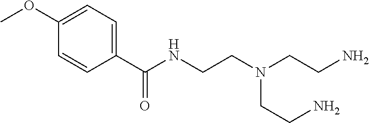 [SMILES: COc1ccc(C(=O)NCCN(CCN)CCN)cc1] |
| | N-(2-(bis(2-aminoethyl)amino)ethyl)-4-methoxybenzamide |
| AA-306 | 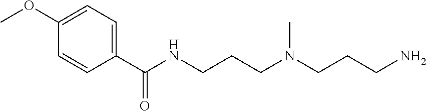 [SMILES: COc1ccc(C(=O)NCCCN(C)CCCN)cc1] |
| | N-(3-((3-aminopropyl)(methyl)amino)propyl)-4-methoxybenzamide |
| AA-113 | 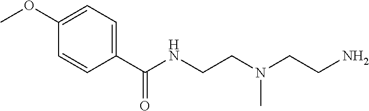 [SMILES: COc1ccc(C(=O)NCCN(C)CCN)cc1] |
| | N-(2-((2-aminoethyl)(methyl)amino)ethyl)-4-methoxybenzamide |
| AA-T3A | 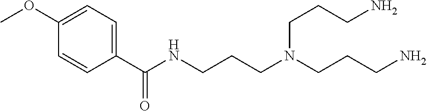 [SMILES: COc1ccc(C(=O)NCCCN(CCCN)CCCN)cc1] |
| | N-(3-(bis(3-aminopropyl)amino)propyl)-4-methoxybenzamide |
| AA-PAMAM | 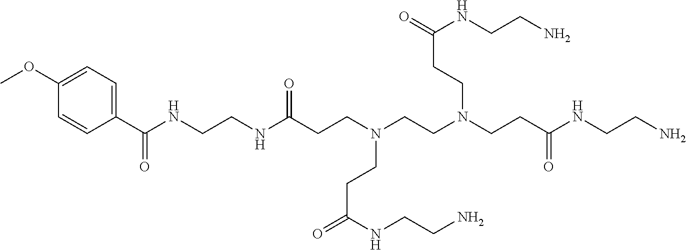 [SMILES: COc1ccc(C(=O)NCCNC(=O)CCN(CCC(=O)NCCN)CCN(CCC(=O)NCCN)CCC(=O)NCCN)cc1] |
| | 3,3′-((2-((3-((2-aminoethyl)amino)-3-oxopropyl)(3-((2-(4- |
| | methoxybenzamido)ethyl)amino)-3- |
| | oxopropyl)amino)ethyl)azanediyl)bis(N-(2-aminoethyl)propanamide) |
| AA-DAB | 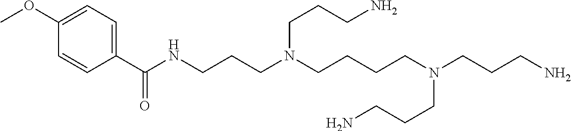 [SMILES: COc1ccc(C(=O)NCCCN(CCCN)CCCCN(CCCN)CCCN)cc1] |
| | N-(3-((3-aminopropyl)(4-(bis(3- |
| | aminopropyl)amino)butyl)amino)propyl)-4-methoxybenzamide |

TABLE 2
Exemplary AA-lipidoids of the present invention

| Name | Structure |
|---|---|
| AA-114- C12 | 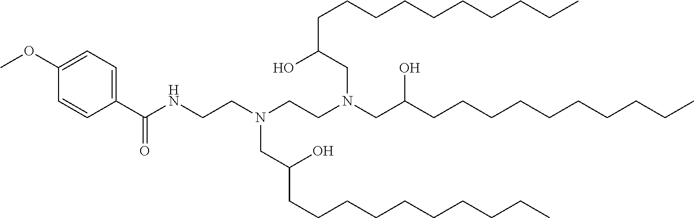 [SMILES: CCCCCCCCCCC(O)CN(CCNC(=O)c1ccc(OC)cc1)CCN(CC(O)CCCCCCCCCC)CC(O)CCCCCCCCCC] |
| | N-(2-((2-(bis(2-hydroxydodecyl)amino)ethyl)(2-hydroxydodecyl)amino)ethyl)-4- |
| | methoxybenzamide |
| AA-200- C12 | 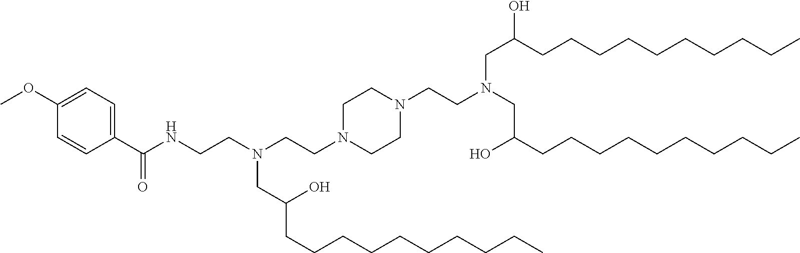 [SMILES: CCCCCCCCCCC(O)CN(CCNC(=O)c1ccc(OC)cc1)CCN1CCN(CCN(CC(O)CCCCCCCCCC)CC(O)CCCCCCCCCC)CC1] |
| | N-(2-((2-(4-(2-(bis(2-hydroxydodecyl)amino)ethyl)piperazin-1-yl)ethyl)(2- |
| | hydroxydodecyl)amino)ethyl)-4-methoxybenzamide |
| AA-110- C12 | 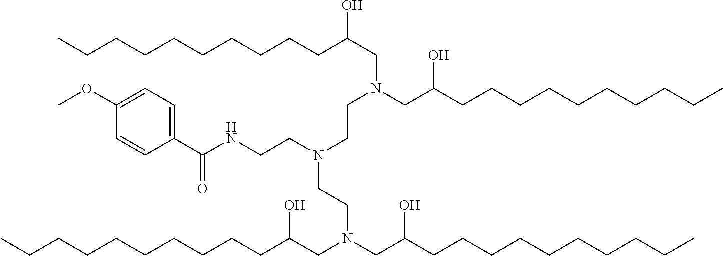 [SMILES: CCCCCCCCCCC(O)CN(CCN(CCNC(=O)c1ccc(OC)cc1)CCN(CC(O)CCCCCCCCCC)CC(O)CCCCCCCCCC)CC(O)CCCCCCCCCC] |
| | N-(2-(bis(2-hydroxydodecyl)amino)ethyl)amino)ethyl)-4- |
| | methoxybenzamide |
| AA-110- C14 | 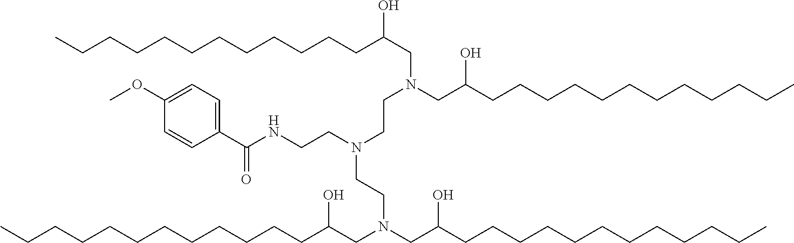 [SMILES: CCCCCCCCCCCCC(O)CN(CCN(CCNC(=O)c1ccc(OC)cc1)CCN(CC(O)CCCCCCCCCCCC)CC(O)CCCCCCCCCCCC)CC(O)CCCCCCCCCCCC] |
| | N-(2-(bis(2-(bis(2-hydroxytetradecyl)amino)ethyl)amino)ethyl)-4- |
| | methoxybenzamide |
| AA-110- C16 | 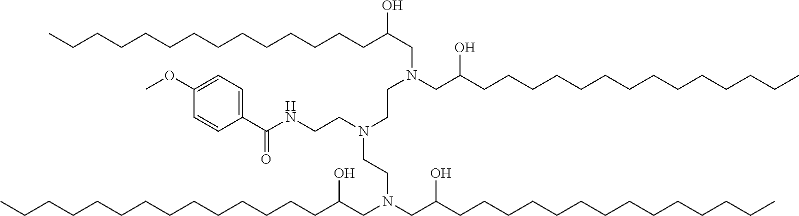 [SMILES: CCCCCCCCCCCCCCC(O)CN(CCN(CCNC(=O)c1ccc(OC)cc1)CCN(CC(O)CCCCCCCCCCCCCC)CC(O)CCCCCCCCCCCCCC)CC(O)CCCCCCCCCCCCCC] |
| | N-(2-(bis(2-(bis(2-hydroxyhexadecyl)amino)ethyl)amino)ethyl)-4- |
| | methoxybenzamide |
| AA-306- C12 | 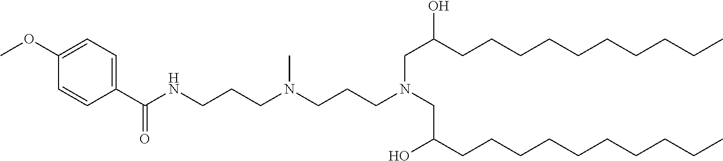 [SMILES: CCCCCCCCCCC(O)CN(CCCN(C)CCCNC(=O)c1ccc(OC)cc1)CC(O)CCCCCCCCCC] |
| | N-(3-((3-(bis(2-hydroxydodecyl)amino)propyl)(methyl)amino)propyl)-4- |
| | methoxybenzamide |
| AA-306- C14 | 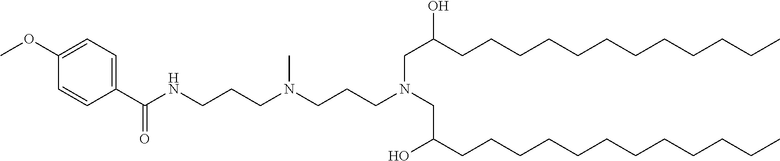 [SMILES: CCCCCCCCCCCCC(O)CN(CCCN(C)CCCNC(=O)c1ccc(OC)cc1)CC(O)CCCCCCCCCCCC] |
| | N-(3-((3-(bis(2-hydroxytetradecyl)amino)propyl)(methyl)amino)propyl)-4- |
| | methoxybenzamide |
| AA-306- C16 | 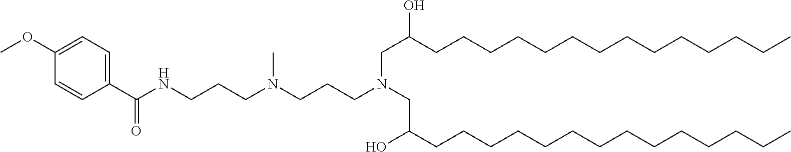 [SMILES: CCCCCCCCCCCCCCC(O)CN(CCCN(C)CCCNC(=O)c1ccc(OC)cc1)CC(O)CCCCCCCCCCCCCC] |
| | N-(3-((3-(bis(2-hydroxyhexadecyl)amino)propyl)(methyl)amino)propyl)-4- |
| | methoxybenzamide |
| AA-113- C12 | 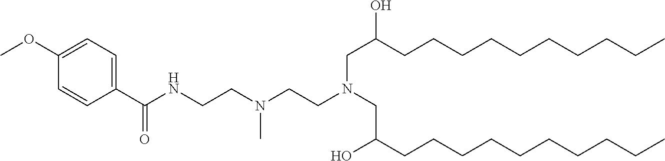 [SMILES: CCCCCCCCCCC(O)CN(CCN(C)CCNC(=O)c1ccc(OC)cc1)CC(O)CCCCCCCCCC] |
| | N-(2-((2-(bis(2-hydroxydodecyl)amino)ethyl)(methyl)amino)ethyl)-4- |
| | methoxybenzamide |
| AA-113- C14 | 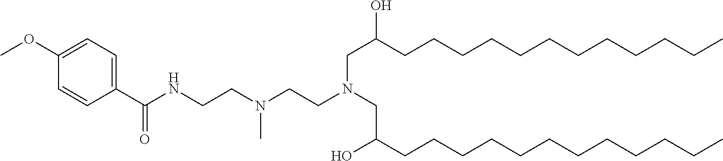 [SMILES: CCCCCCCCCCCCC(O)CN(CCN(C)CCNC(=O)c1ccc(OC)cc1)CC(O)CCCCCCCCCCCC] |
| | N-(2-((2-(bis(2-hydroxytetradecyl)amino)ethyl)(methyl)amino)ethyl)-4- |
| | methoxybenzamide |
| AA-113- C16 | 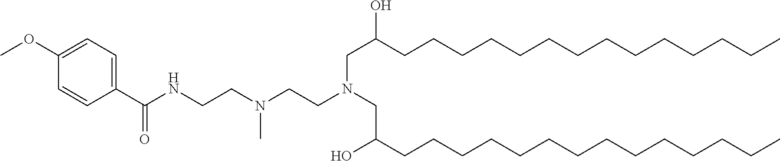 [SMILES: CCCCCCCCCCCCCCC(O)CN(CCN(C)CCNC(=O)c1ccc(OC)cc1)CC(O)CCCCCCCCCCCCCC] |
| | N-(2-((2-bis(2-hydroxyhexadecyl)amino)ethyl)(methyl)amino)ethyl)-4- |
| | methoxybenzamide |
| AA-T3A- C12 | 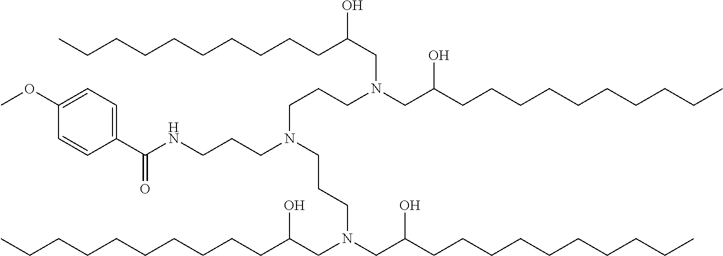 [SMILES: CCCCCCCCCCC(O)CN(CCCN(CCCNC(=O)c1ccc(OC)cc1)CCCN(CC(O)CCCCCCCCCC)CC(O)CCCCCCCCCC)CC(O)CCCCCCCCCC] |
| | N-(3-(bis(3-(bis(2-hydroxydodecyl)amino)propyl)amino)propyl)-4- |
| | methoxybenzamide |
| AA-T3A- C14 | 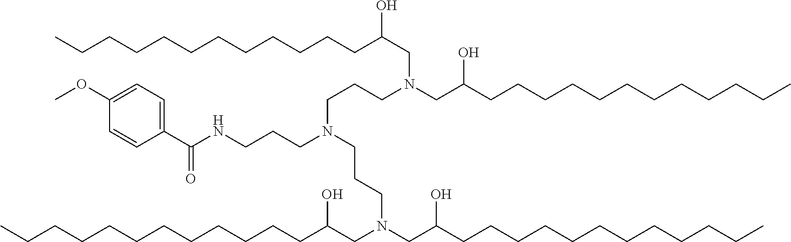 [SMILES: CCCCCCCCCCCCC(O)CN(CCCN(CCCNC(=O)c1ccc(OC)cc1)CCCN(CC(O)CCCCCCCCCCCC)CC(O)CCCCCCCCCCCC)CC(O)CCCCCCCCCCCC] |
| | N-(3-(bis(3-(bis(2-hydroxytetradecyl)amino)propyl)amino)propyl)-4- |
| | methoxybenzamide |
| AA-T3A- C16 | 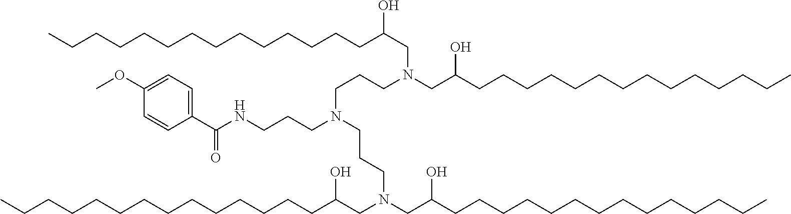 [SMILES: CCCCCCCCCCCCCCC(O)CN(CCCN(CCCNC(=O)c1ccc(OC)cc1)CCCN(CC(O)CCCCCCCCCCCCCC)CC(O)CCCCCCCCCCCCCC)CC(O)CCCCCCCCCCCCCC] |
| | N-(3-(bis(3-(bis(2-hydroxyhexadecyl)amino)propyl)amino)propyl)-4- |
| | methoxybenzamide |
| AA- PAMAM- C12 | 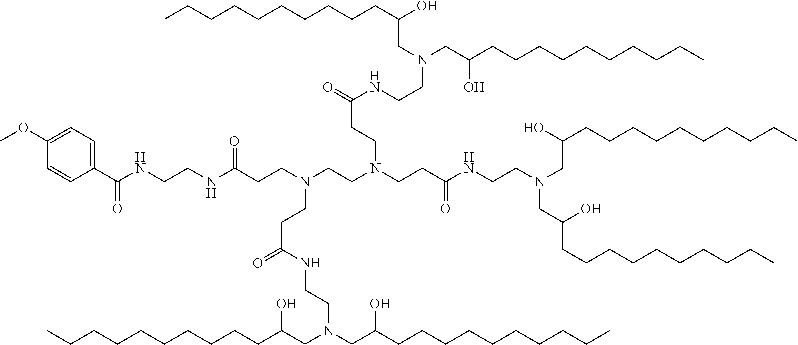 [SMILES: CCCCCCCCCCC(O)CN(CCNC(=O)CCN(CCC(=O)NCCNC(=O)c1ccc(OC)cc1)CCN(CCC(=O)NCCN(CC(O)CCCCCCCCCC)CC(O)CCCCCCCCCC)CCC(=O)NCCN(CC(O)CCCCCCCCCC)CC(O)CCCCCCCCCC)CC(O)CCCCCCCCCC] |
| | 3,3′-((2-((3-((2-(bis(2-hydroxydodecyl)amino)ethyl)amino)-3-oxopropyl)(3-((2- |
| | (4-methoxybenzamido)ethyl)amino)-3-oxopropyl)amino)ethyl)azanediyl)bis(N- |
| | (2-(bis(2-hydroxydodecyl)amino)ethyl)propanamide) |
| AA- PAMAM- C14 | 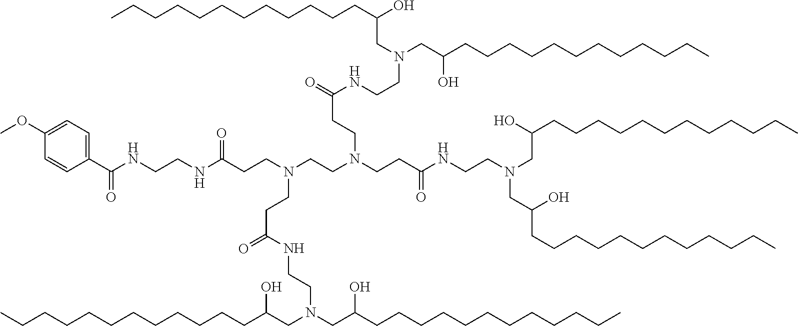 [SMILES: CCCCCCCCCCCCC(O)CN(CCNC(=O)CCN(CCC(=O)NCCNC(=O)c1ccc(OC)cc1)CCN(CCC(=O)NCCN(CC(O)CCCCCCCCCCCC)CC(O)CCCCCCCCCCCC)CCC(=O)NCCN(CC(O)CCCCCCCCCCCC)CC(O)CCCCCCCCCCCC)CC(O)CCCCCCCCCCCC] |
| | 3,3′-((2-((3-((2-(bis(2-hydroxytetradecyl)amino)ethyl)amino)-3-oxopropyl)(3-((2- |
| | (4-methoxybenzamido)ethyl)amino)-3-oxopropyl)amino)ethyl)azanediyl)bis(N- |
| | (2-(bis(2-hydroxytetradecyl)amino)ethyl)propanamide) |
| AA- PAMAM- C16 | 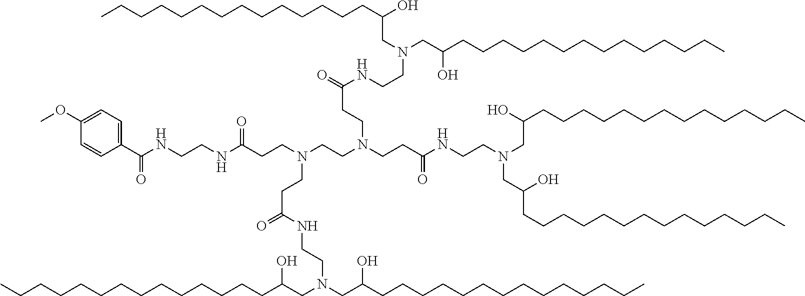 [SMILES: CCCCCCCCCCCCCCC(O)CN(CCNC(=O)CCN(CCC(=O)NCCNC(=O)c1ccc(OC)cc1)CCN(CCC(=O)NCCN(CC(O)CCCCCCCCCCCCCC)CC(O)CCCCCCCCCCCCCC)CCC(=O)NCCN(CC(O)CCCCCCCCCCCCCC)CC(O)CCCCCCCCCCCCCC)CC(O)CCCCCCCCCCCCCC] |
| | 3,3′-((2-((3-((2-(bis(2-hydroxyhexadecyl)amino)ethyl)amino)-3-oxopropyl)(3-((2- |
| | (4-methoxybenzamido)ethyl)amino)-3-oxopropyl)amino)ethyl)azanediyl)bis(N- |
| | (2-(bis(2-hydroxyhexadecyl)amino)ethyl)propanamide) |
| AA-DAB- C12 | 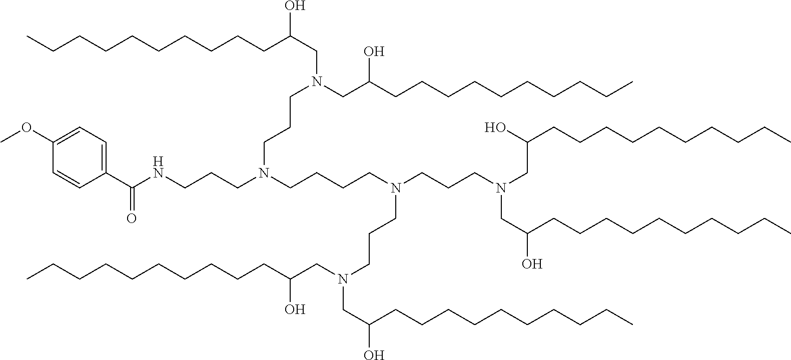 [SMILES: CCCCCCCCCCC(O)CN(CCCN(CCCCN(CCCN(CC(O)CCCCCCCCCC)CC(O)CCCCCCCCCC)CCCN(CC(O)CCCCCCCCCC)CC(O)CCCCCCCCCC)CCCNC(=O)c1ccc(OC)cc1)CC(O)CCCCCCCCCC] |
| | N-(3-((3-(bis(2-hydroxydodecyl)amino)propyl)(4-(bis(3-(bis(2- |
| | hydroxydodecyl)amino)propyl)amino)butyl)amino)propyl)-4-methoxybenzamide |
| AA-DAB- C14 | 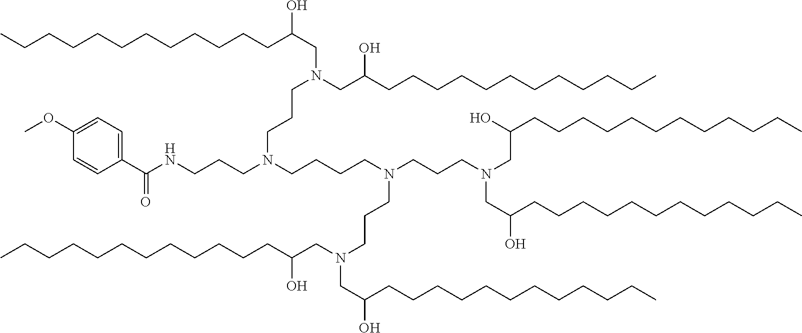 [SMILES: CCCCCCCCCCCCC(O)CN(CCCN(CCCCN(CCCN(CC(O)CCCCCCCCCCCC)CC(O)CCCCCCCCCCCC)CCCN(CC(O)CCCCCCCCCCCC)CC(O)CCCCCCCCCCCC)CCCNC(=O)c1ccc(OC)cc1)CC(O)CCCCCCCCCCCC] |
| | N-(3-((3-(bis(2-hydroxytetradecyl)amino)propyl)(4-(bis(3-(bis(2- |
| | hydroxytetradecyl)amino)propyl)amino)butyl)amino)propyl)-4- |
| | methoxybenzamide |
| AA-DAB- C16 | 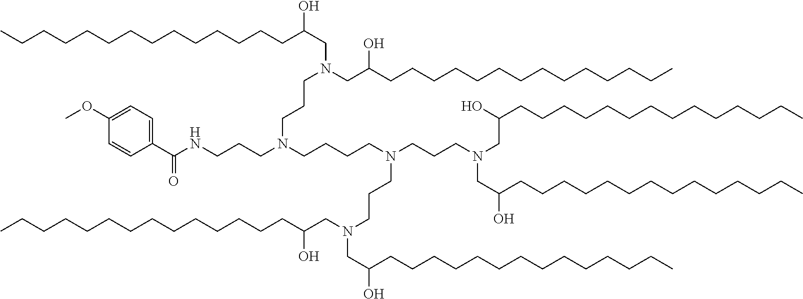 [SMILES: CCCCCCCCCCCCCCC(O)CN(CCCN(CCCCN(CCCN(CC(O)CCCCCCCCCCCCCC)CC(O)CCCCCCCCCCCCCC)CCCN(CC(O)CCCCCCCCCCCCCC)CC(O)CCCCCCCCCCCCCC)CCCNC(=O)c1ccc(OC)cc1)CC(O)CCCCCCCCCCCCCC] |
| | N-(3-((3-(bis(2-hydroxyhexadecyl)amino)propyl)(4-(bis(3-(bis(2- |
| | hydroxyhexadecyl)amino)propyl)amino)butyl)amino)propyl)-4- |
| | methoxybenzamide |

General Method for the Synthesis of Lipidoids without Anisamide

Lipidoids without anisamide were synthesized by reacting excessive alkyl epoxides (e.g., 7.2 equiv. for T3A core) with polyamines at 80° C. for 2 days as previously reported.

### LNP Formulation

An organic phase was prepared in ethanol by solubilizing ionizable lipid (lipidoid, AA-lipidoid, or MC3), DSPC, cholesterol and C14-PEG2000 at a molar ratio of 50:10:38.5:1.5. The aqueous phase was prepared in 10 mM citrate buffer (pH 3) with siRNA or luciferase mRNA. The aqueous phase and organic phase were mixed at an ionizable lipid:RNA weight ratio of 10:1 and at a flow rate of 1.8 mL/min and 0.6 mL/min (3:1) using Pump 33 DDS syringe pumps (Harvard Apparatus, MA) in a microfluidic device with a staggered herringbone micromixer design. The microfluidic devices were fabricated in polydimethylsiloxane according to standard soft lithographic procedures. A two-step exposure process was used to create the SU-8 master with positive channel features on a silicon wafer. Each mixing channel is 4 cm in length. LNPs were dialyzed against 1×PBS in a 20 kDa MWCO cassette for 2 h, filtered through a 0.22 μM filter, and stored at 4° C. For in vitro screening, LNPs were prepared by pipette mixing and directly used to treat cells without further dialysis.

### LNP Characterization

The hydrodynamic diameter, polydispersity index (PDI), and zeta potential of LNPs were measured using a Zetasizer Nano ZS90 (Malvern Instruments, Malvern, UK). The morphology of LNPs was characterized by transmission electron microscopy (JEOL 1010, Tokyo, Japan). The siRNA-LNP complex was analyzed by agarose gel electrophoresis. siRNA encapsulation efficiency was determined using a modified Quant-iT RiboGreen RNA assay (Invitrogen) as previously described. The pKa of LNP was determined by 6-(p-toluidinyl)naphthalene-2-sulfonic acid (TNS) assay as previously described.

### Cell Culture and Animal Studies

A murine NIH 3T3 fibroblast cell line was used and obtained from the University of Pennsylvania. The NIH 3T3-GFP cell line was purchased from Cell Biolabs (#AKR-214). The primary murine HSCs were obtained from Sciencell (cat. M5300-57). The immortalized primary murine H2.35 hepatocyte cell line was obtained from the University of Pennsylvania. The murine bEnd.3 endothelial cell line and the murine RAW264.7 macrophage cell line were obtained from the American Type Culture Collection (ATCC). Primary HSCs were maintained in Stellate Cell Medium (#5301, Sciencell) and used between passage 3 and 8. All other cells were maintained in Dulbecco's Modified Eagle Medium (DMEM) supplemented with 10% fetal bovine serum (FBS), 100 U/mL penicillin and 100 μg/mL streptomycin at 37° C. in a humidified incubator of 5% CO₂, and routinely tested for mycoplasma contamination. Activated 3T3 fibroblasts and HSCs were obtained by stimulation with 10 ng/ml of TGF-β for 24 h.

Balb/c male mice (6-8 weeks age, 22-25 g body weight) were purchased from Jackson Laboratory. Liver fibrosis was induced by intraperitoneal (i.p.) injection of 20% CCl₄ (0.7 μl/g) in corn oil twice a week for 4 weeks.

### High-Throughput Screening of LNPs In Vitro

High-throughput screening of LNPs was performed by evaluating GFP knockdown efficiency in 96-well plates using a plate reader assay. 3T3-GFP cells were re-suspended in DMEM containing 10 ng/ml of TGF-β, and seeded into a 96-well plate at a density of 5,000 cells per well. After 24 h, activated cells were treated with various LNPs loaded with siGFP (50 nM). At 48 h post-treatment, the GFP signal in each well was obtained using an Infinite M Plex plate reader (Tecan, Morrisville, NC, USA) with an excitation of 488 nm and an emission of 520 nm. Cell viability was measured using a CellTiter-Glo Luminescent Cell Viability Assay according to the manufacturer's instruction (G7572, Promega).

The top-performing LNPs were subjected to a second round of screening by evaluating their dependency on sigma receptor-mediated transfection. Activated 3T3-GFP cells were treated with or without haloperidol (30 μM) for 2 h before various LNPs loaded with siGFP (50 nM) were used to treat these cells for another 48 h. The GFP signal in each well was obtained as described above.

Cellular Uptake and mRNA Transfection

Activated 3T3-GFP cells in a 6-well plate (5×10⁵ per well) were pre-treated with or without haloperidol (30 μM) for 2 h before treatment with AA-T3A-C12 LNP loaded with Cy5-siRNA (50 nM). At 6 h post-treatment, cells were collected for analysis using flow cytometry (BD, LSR II).

3T3-GFP cells (2.5×10⁵ per well) and H2.35 cells (2.5×10⁵ per well) were co-cultured in a 6-well plate and stimulated with TGF-β for 24 h. Cells were treated with AA-T3A-C12 LNP, T3A-C12 LNP or MC3 LNP loaded with Cy5-siRNA (50 nM) for 6 h before collection for flow cytometry analysis.

Activated 3T3-GFP cells or H2.35 cells in a 96-well plate (5,000 per well) were treated with AA-T3A-C12 LNP or MC3 LNP loaded with luciferase mRNA (15 ng/well) for 24 h. Luciferase expression was evaluated by Luciferase Reporter 1000 Assay System according to the manufacturer's protocol (E4550, Promega), and cell viability was measured using a CellTiter-Glo Luminescent Cell Viability Assay.

### Dose- and Time-Independent GFP Knockdown In Vitro

Activated 3T3-GFP cells in a 96-well plate (5,000 per well) were treated with AA-T3A-C12/siGFP LNP at siRNA concentrations ranging from 25 to 100 nM. GFP expression was evaluated by a plate reader at 24 and 48 h post-transfection. Cell viability was measured as described elsewhere herein.

To evaluate GFP knockdown efficiency using flow cytometry, activated 3T3-GFP cells in a 6-well plate (5×10⁵ per well) were treated with AA-T3A-C12/siGFP LNP at siRNA concentrations ranging from 25 to 100 nM for 48 h. Cells were collected for analysis by flow cytometry.

### Western Blot Analysis

20 μg of cell protein was loaded for electrophoresis. Blots were incubated with HSP47 antibody (1:1,000, #NBP1-97491, Novus Biologicals) or Sigma Receptor antibody (1:200, #sc-137075, Santa Cruz) overnight at 4° C. After being washed 3 times, blots were incubated with IRDye 800CW donkey anti-mouse IgG secondary antibody (#925-32212, LiCor) at a 1:10,000 dilution for 1 h at RT. Blots were imaged using an Odyssey IR Imaging System. GAPDH was used an internal control.

### HSP47 Knockdown In Vitro

Activated 3T3 cells in 35-mm glass-bottom dishes were treated with AA-T3A-C12/siGFP LNP, AA-T3A-C12/siHSP47 LNP or MC3/siHSP47 LNP (50 nM). 48 h later, cells were subjected to immunofluorescence staining of HSP47 using an Immunofluorescence Application Solutions Kit (#12727, CST). Samples were incubated with HSP47 antibody at a 1:200 dilution overnight. After being washed 3 times, samples were incubated with Alexa Fluor® 488 conjugated goat anti-mouse IgG (H+L), F(ab′)₂ fragment antibody (#4408, CST) at a 1:1,000 dilution for 1 h. Nuclei were stained with Hoechst 33342 (10 μg/mL) before images were taken using a confocal laser scanning microscope (LSM 710, Zeiss).

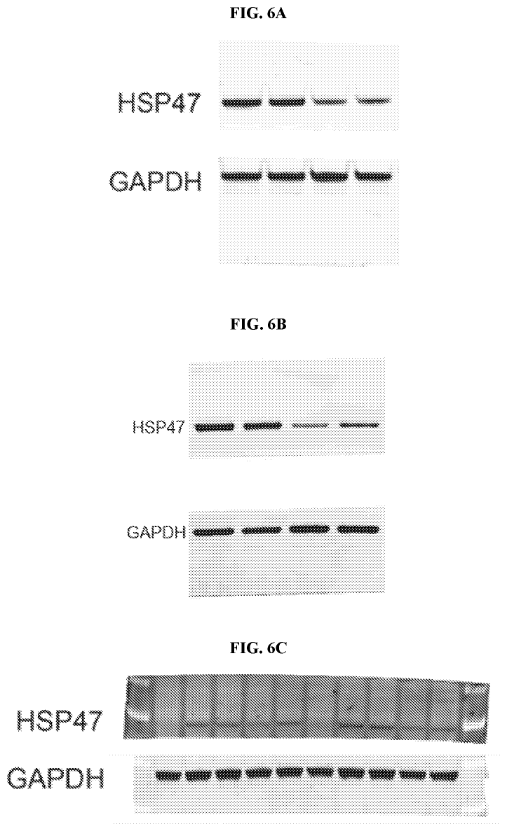

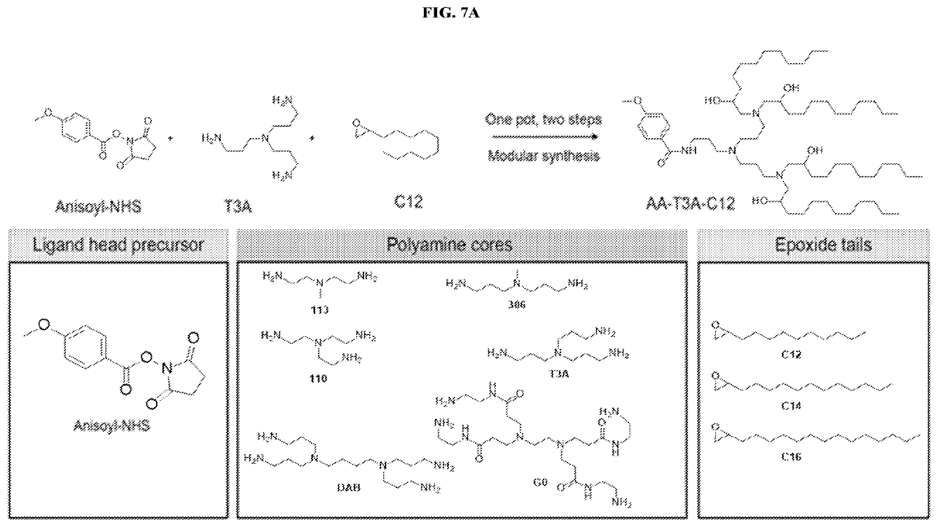

To evaluate HSP47 knockdown by Western blot, activated 3T3 cells or HSCs in a 6-well plate (5×10⁵ per well) were treated with AA-T3A-C12/siGFP LNP, AA-T3A-C12/siHSP47 LNP or MC3/siHSP47 LNP (50 nM) for 48 h. Cells were harvested for Western blot analysis. Uncropped blots were shown in FIG. 6.

### LNP Biodistribution

AA-T3A-C12 LNP or MC3 LNP loaded with Cy5-siRNA (5 μg/mouse) was i.v. injected into each fibrotic mouse (n=3). 1 h post-injection, mice were euthanized and major organs were harvested for ex vivo imaging using an IVIS imaging system (PerkinElmer). Livers were collected to prepare 10 μm cryosections. Samples were stained with FITC-conjugated α-smooth muscle actin antibody (#F3777, Sigma) at a 1:500 dilution overnight at 4° C. Nuclei were stained with Hoechst 33342 (10 μg/mL) before images were taken using a confocal laser scanning microscope.

### Therapeutic Studies

Mice were treated with CCl₄ twice weekly for 4 weeks. On week 3, mice (n=5) were treated with PBS, AA-T3A-C12/siGFP LNP, AA-T3A-C12/siHSP47 LNP or MC3/siHSP47 LNP (5 μg siRNA/mouse) twice weekly for 2 weeks. Untreated (healthy) mice (n=5) were used as a control group. Body weight was recorded twice a week during the experiment. Two days after the last treatment, mice were anesthetized and blood was collected through the retro-orbital route. Serum samples were prepared and stored at −80° C. until use. Major organs (heart, liver, spleen, lung and kidney) were collected. A part of liver was collected for cryosectioning and subjected to immunofluorescence staining of HSP47 as described above. A part of liver was homogenized in RIPA buffer (Thermo Scientific) and proteins were extracted for Western blot analysis of HSP47 expression as described above. Uncropped blots were shown in FIGS. 6A-6C.

### Histological and Blood Biochemical Analysis

Major organs were fixed at 4% paraformaldehyde, embedded in paraffin, cut into 5 μm sections and stained with hematoxylin and eosin (H&E) for pathological analysis. Liver sections were also stained with Picrosirius Red (ab150681, Abcam). Images were taken using a microscope (FL Auto 2 Imaging System, EVOS).

Liver toxicities were evaluated by measuring aspartate aminotransferase (#701640, Cayman), alanine aminotransferase (#700260, Cayman), total bilirubin (#701720, Cayman) in the serum. The immunotoxicity was evaluated by measuring serum IL-6 (#88-7064, Invitrogen) and TNF-α (#88-7324, Invitrogen) using enzyme linked immunosorbent assay.

### Statistical Analysis

Data are presented as mean±SD. Student's t-test or one-way analysis of variance (ANOVA) followed by Tukey test was applied for comparison between two groups or among multiple groups, respectively. p<0.05 was considered to be statistically significant.

### Example 1: Design and Synthesis of AA-Lipidoids

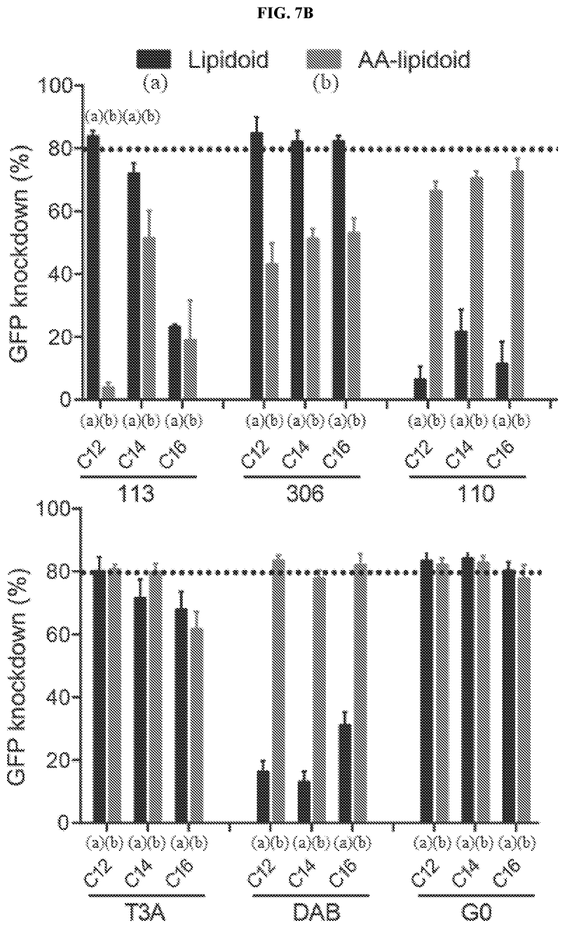

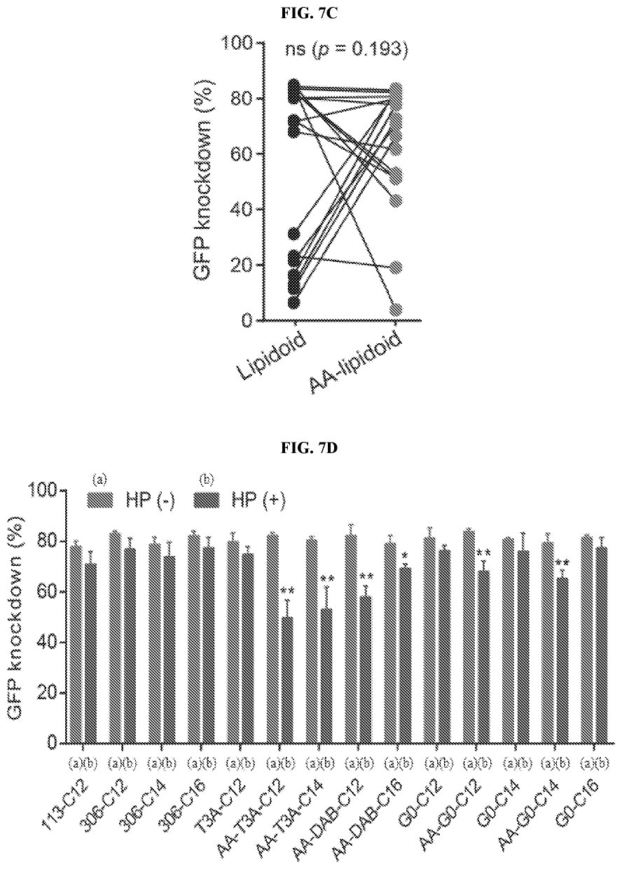

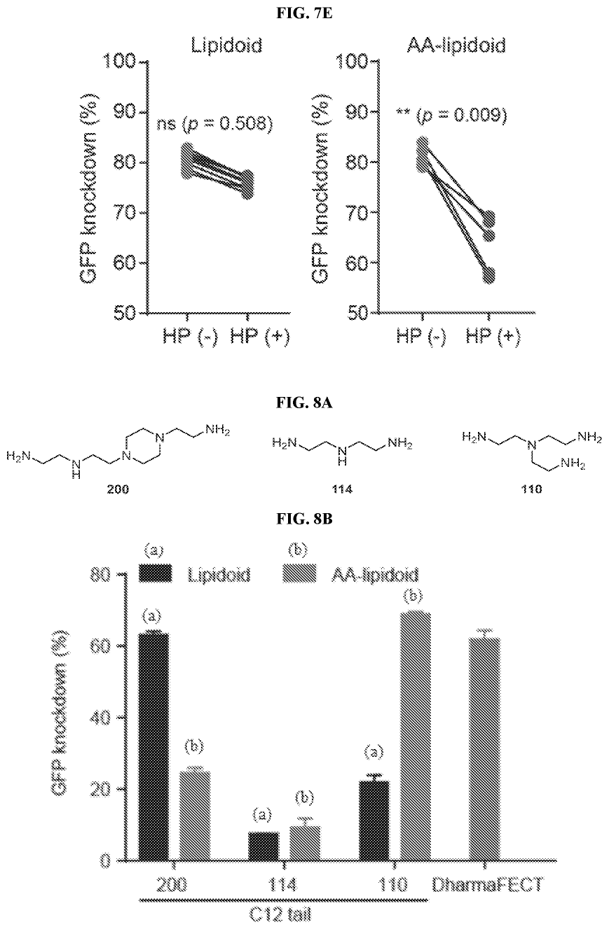

In order to incorporate anisamide into lipidoids and enable parallel synthesis of a series of AA-lipidoids, a one-pot, two-step synthetic method was developed through the combination of an amine-succinimide coupling reaction and a ring-opening reaction (i.e., S_{N}2) of an epoxide and an amine, due to the simplicity and compatibility of these two reactions. Briefly, the targeting ligand precursor anisoyl-N-hydroxysuccinimide (anisoyl-NHS, FIGS. 2-3) was coupled to a polyamine core via an amide bond, wherein the free amines were subsequently substituted with alkyl chains by addition to an alkyl-substituted epoxide tail (FIG. 7A). Such a tandem and modular synthetic strategy dramatically simplifies synthesis, and the resultant materials can be used directly for in vitro screening without purification.

### Example 2: In Vitro Screening of AA-Lipidoids

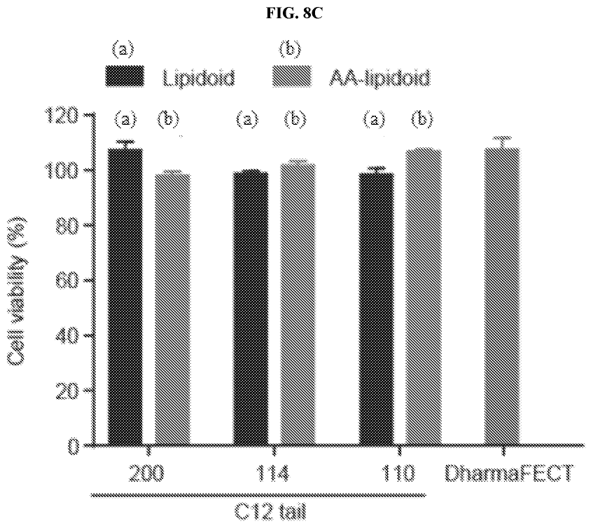

Initially, three representative polyamine (i.e., piperazine derivative 200, linear amine 114, and branched amine 110)-derived lipidoids, with or without anisamide incorporation, were evaluated for in vitro GFP silencing to assess which types of polyamines warranted further investigation (FIG. 8A-8C). Activated 3T3-GFP fibroblasts were obtained by pro-fibrotic transforming growth factor-β (TGF-β) stimulation, which spontaneously overexpressed sigma receptors. Pilot screening of epoxide Cl₂-tailed lipidoids and AA-lipidoids showed that the branched amine 110-derived AA-110-C12 mediated ˜70% GFP knockdown in activated 3T3-GFP fibroblasts, outperforming its counterpart lipidoid 110-C12 without anisamide as well as two benchmarks, 200-C12 (also known as C12-200) and the commercially available DharmaFECT transfection reagent.

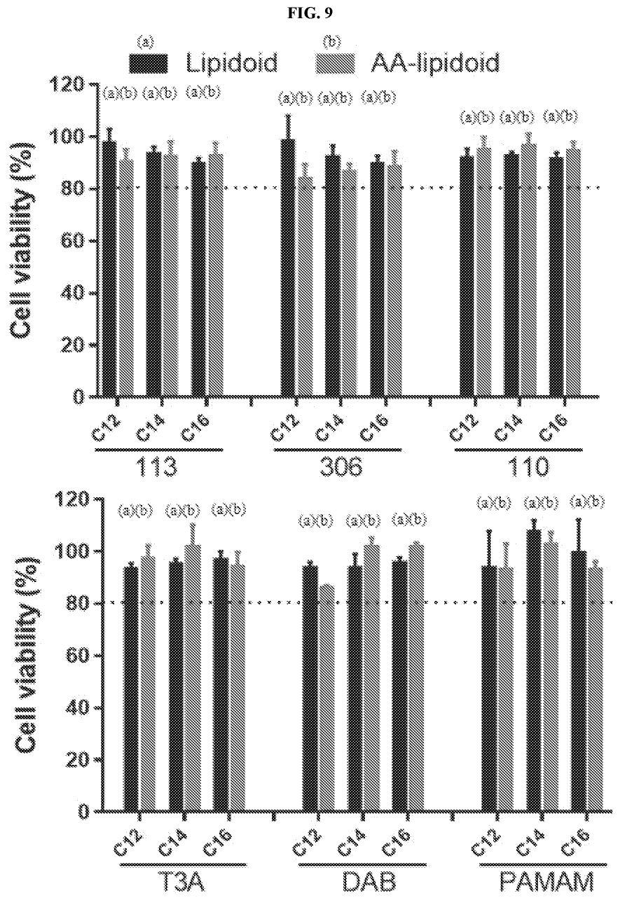

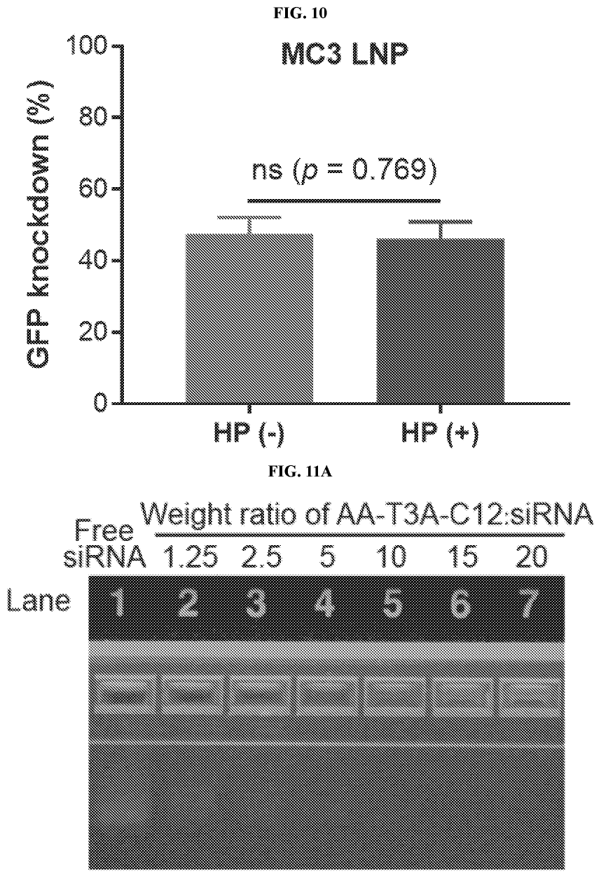

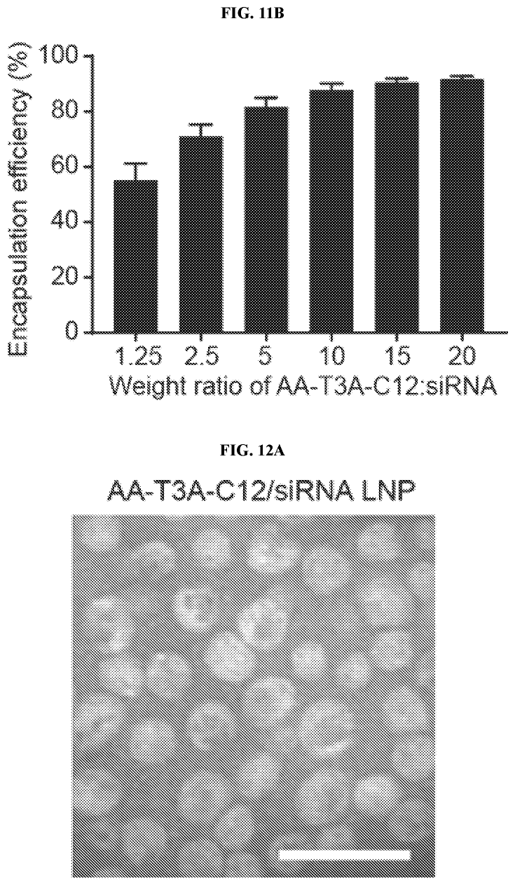

Therefore, additional branched polyamines and epoxide tails were incorporated to expand the AA-lipidoid library (FIG. 7A). In total, 18 AA-lipidoids and 18 counterpart lipidoids were synthesized, formulated into 36 LNPs along with excipients (e.g., cholesterol, DSPE, and C14-PEG) and GFP siRNA (i.e., siGFP), and subjected to high-throughput screening in activated 3T3-GFP fibroblasts. No significant cytotoxicity was observed for all LNPs after 48 h treatment (FIG. 9). Interestingly, less branched polyamine 113 and 306-derived AA-lipidoids showed reduced knockdown efficiency compared to their counterpart lipidoids regardless of epoxide tails, while the more branched polyamine 110, T3A, DAB and G0-derived AA-lipidoids either maintained or dramatically enhanced knockdown efficiency (FIG. 7B). Without wishing to be bound by theory, a possible explanation for this difference is that, since 113 and 306 cores comprise less amines, consumption of a primary amine by anisamide conjugation more profoundly affects the structure and ionization of the resultant LNP than other cores with more amines. Nevertheless, an analysis of structure-activity relationships demonstrated that incorporation of anisamide did not compromise the overall potency of lipidoids (FIG. 7C). In this round of screening, 6 AA-lipidoids and 8 lipidoids were identified as highly potent, with GFP knockdown efficiencies above 80%.

These top 14 lipidoid candidates achieving >80% GFP knockdown were then subjected to a second-round of screening to evaluate their dependency on sigma receptor-mediated transfection. Haloperidol (HP), a sigma receptor antagonist, was used to treat activated 3T3-GFP fibroblasts before LNPs treatment. For lipidoids without anisamide, no obvious loss of silencing activity was observed after HP treatment (p=0.508, FIGS. 7D-7E). Similar results were observed when the FDA-approved MC3 LNP formulation was tested (Table 3 and FIG. 10). However, all AA-lipidoids showed significantly decreased knockdown efficiency after HP treatment (p=0.009, FIGS. 7D-7E). Among the 6 AA-lipidoids, AA-T3A-C12 demonstrated the most significant loss of activity after sigma receptor blocking, which was chosen for subsequent studies due to its high dependency on sigma receptor-mediated transfection. These results suggest that after a first round of screening for efficiency and a second round of screening for selectivity, a potent AA-lipidoid (AA-T3A-C12) with active targeting ability was identified for further investigation.

TABLE 3
Characterization of Exemplary LNPs

| | Diameter | | Zeta potential | Encapsulation | |
|---|---|---|---|---|---|
| LNP | (nm) | PDI | (mV) | efficiency (%) | pKa |
| Empty AA-T3A-C12 | 59.3 ± 2.5 | 0.021 | −0.7 ± 0.9 | n/a | n/a |
| LNP | | | | | |
| AA-T3A-C12/siRNA | 65.6 ± 1.2 | 0.018 | −1.1 ± 1.5 | 87.4 ± 3.8 | 5.72 |
| LNP | | | | | |
| T3A-C12/siRNA LNP | 64.9 ± 1.8 | 0.034 | −2.2 ± 1.8 | 33.6 ± 8.5 | 5.81 |
| MC3/siRNA LNP | 60.9 ± 3.4 | 0.090 | −1.5 ± 2.2 | 90.1 ± 2.5 | 6.44 |

### Example 3: Characterization of AA_T3A-C12 and LNPs

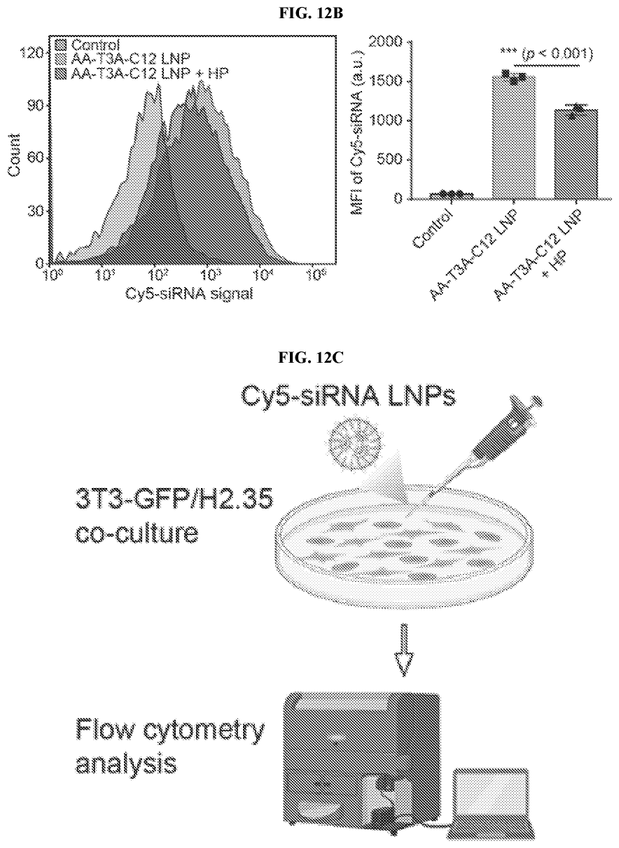

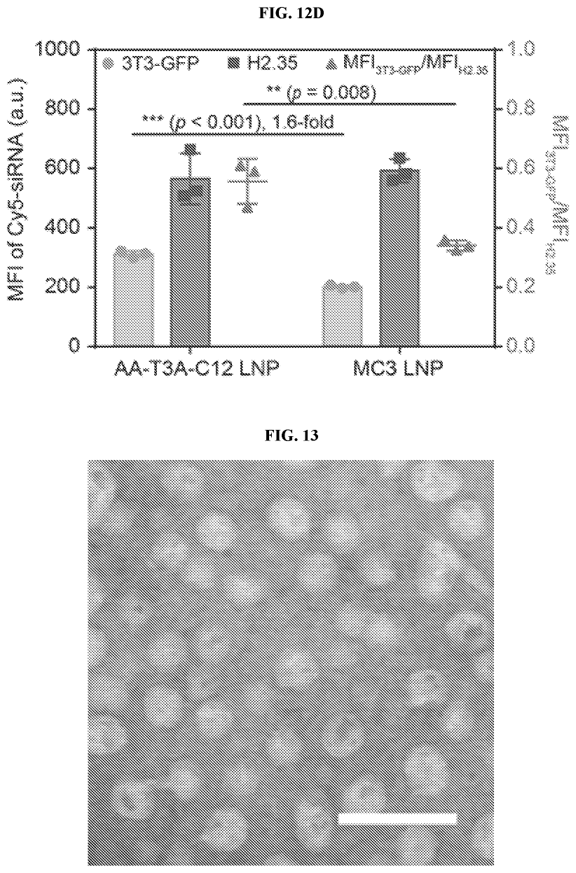

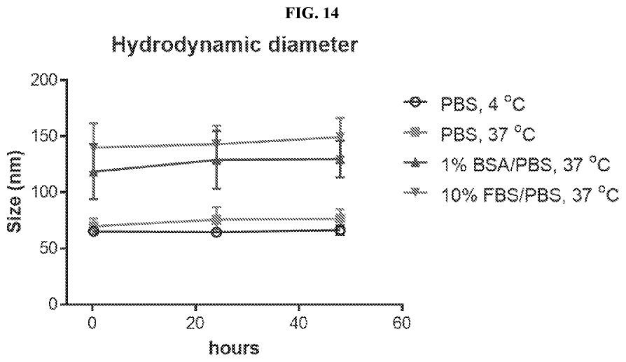

The modularly synthesized lead lipidoid AA-T3A-C12 comprises one anisamide head and four epoxide C12 tails, which are bound by the T3A core (FIG. 7A). The structure of purified AA-T3A-C12 was confirmed by mass spectrometry and proton nuclear magnetic resonance (FIGS. 4-5). Afterwards, the four-component AA-T3A-C12/siRNA LNP was formulated by microfluidic mixing at an ionizable lipid:siRNA weight ratio of 10:1. This weight ratio was chosen based on the results of both the gel retardation assay and the RiboGreen RNA assay, which resulted in high siRNA encapsulation efficiency (EE=87.4 f 3.8%, FIGS. 11A-11B and Table 3). The hydrodynamic diameter of these LNPs was approximately 65.6 nm with a narrow polydispersity index (PDI) of 0.018, which was slightly larger than empty LNPs (Table 3). Moreover, these LNPs had a neutral surface charge and a pKa of 5.72. Transmission electron microscopy (TEM) images showed that both empty LNPs and siRNA-loaded LNPs had a spherical morphology (FIG. 12A and FIG. 13), with some collapsed LNPs due to the dehydration process of sample preparation. Additionally, these LNPs had a high colloidal stability, as large LNP aggregates (>200 nm) were not observed in the protein- or serum-supplemented environment at 37° C. for 48 h (FIG. 14). Overall, the above results suggest the formation of homogeneous and stable LNPs with high siRNA EE that are promising for potent delivery in vitro and in vivo due to their small size (<100 nm), neutral charge, and suitable pKa (5.5-7.0).

### Example 4: Targeted RNA Delivery to Activated Fibroblasts Using AA-T3A-C12 LNP

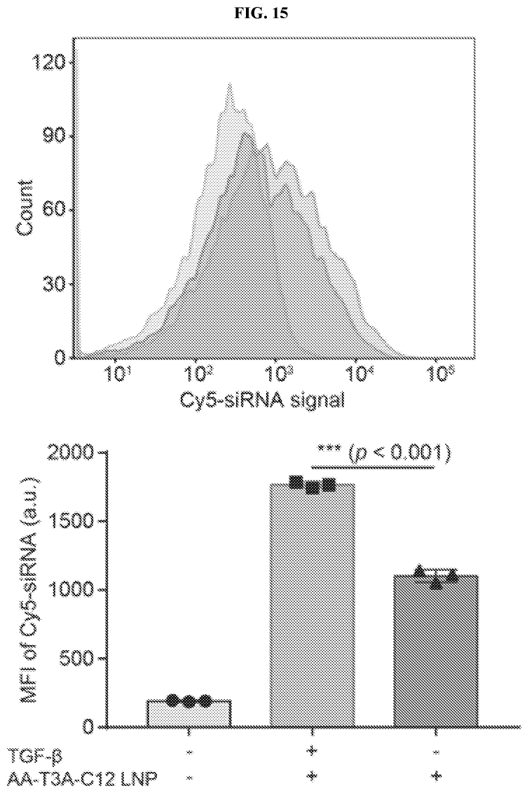

Next, the cellular uptake of AA-T3A-C12 LNPs was analyzed in activated fibroblasts using Cy5-siRNA as a cargo. Flow cytometry analysis showed that the cellular uptake of AA-T3A-C12 LNP was dramatically reduced after HP treatment (p<0.001, FIG. 12B), which explained its reduced gene knockdown efficacy after sigma receptor blockade in FIG. 7D. Moreover, the cellular uptake of AA-T3A-C12 LNP was greater in TGF-β-stimulated 3T3 fibroblasts with overexpressed sigma receptors than that in unstimulated 3T3 fibroblasts (p<0.001, FIG. 15), further confirming the enhanced uptake of AA-T3A-C12 LNP by activated fibroblasts.

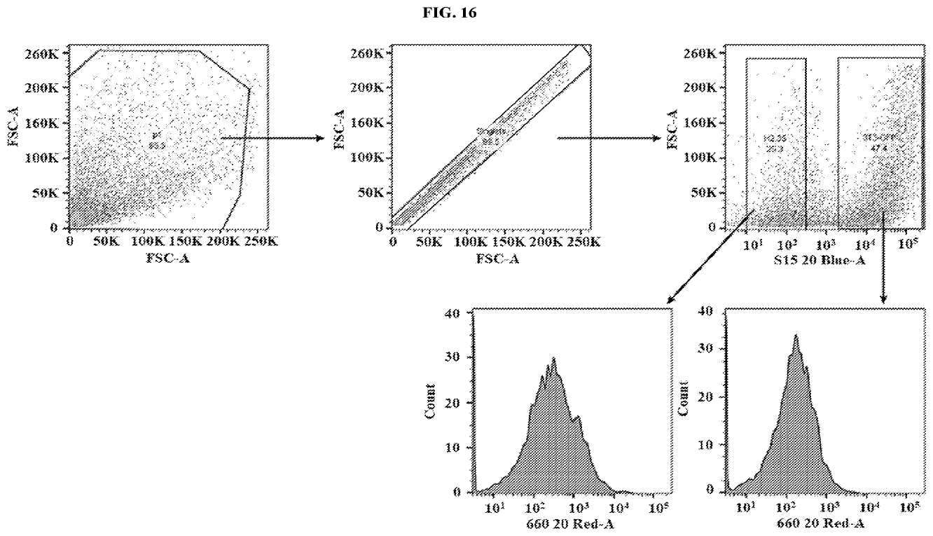

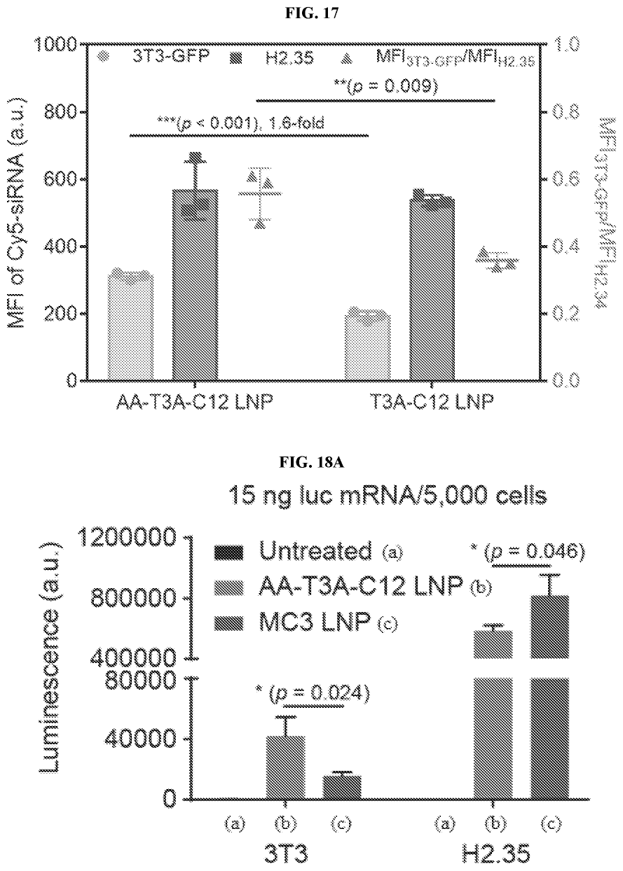

Targeted delivery of AA-T3A-C12 LNP to activated fibroblasts was further investigated in a fibroblast/hepatocyte (3T3-GFP/H2.35) co-culture environment (FIG. 12C and FIG. 16), which was intended to mimic the competitive cellular uptake of LNPs in fibrotic liver by these two cell populations. MC3 LNP was included as a benchmark control. Flow cytometry analysis of cellular uptake showed that AA-T3A-C12 LNP achieved 1.6-fold greater Cy5-siRNA delivery to activated fibroblasts compared to MC3 LNP (p<0.001), but both LNPs achieved similar delivery to hepatocytes (FIG. 12C). Additionally, AA-T3A-C12 LNP outperformed its non-targeted counterpart T3A-C12 LNP in delivering Cy5-siRNA to activated fibroblasts but not hepatocytes (FIG. 17). The calculated mean fluorescence intensity ratio between fibroblast and hepatocyte (MFI_{3T3-GFP}/MFI_{H2.35}, an indicator of fibroblast selectivity) was 0.34 for MC3 LNP, but a much higher ratio of 0.56 was observed for AA-T3A-C12 LNP (p=0.008, FIG. 12C).

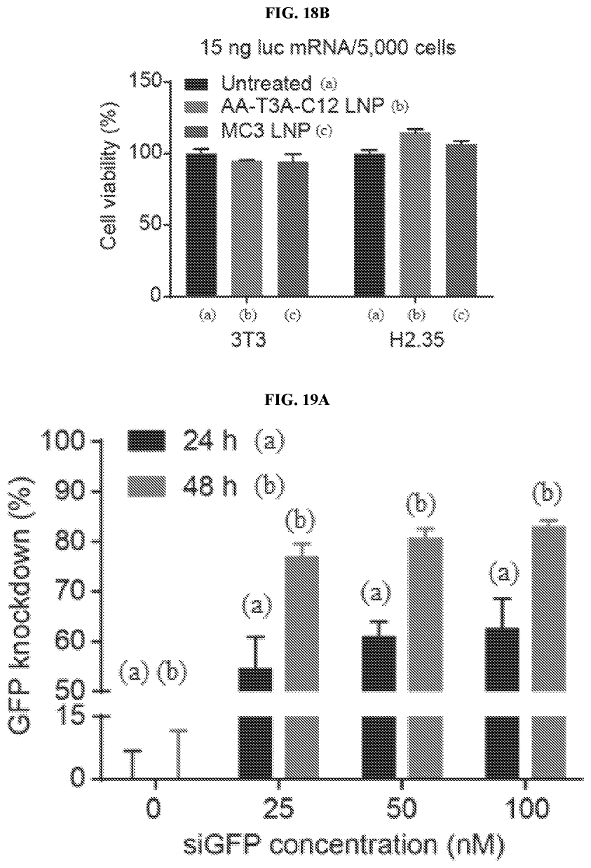

Given that fibroblasts are difficult to transfect and reluctant to engulf foreign substances in comparison to hepatocytes, a ratio of MFI_{3T3-GFP}/MFI_{H2.35} below 1 is expected. Nevertheless, the above results suggest AA-T3A-C12 LNP has better fibroblast selectivity and mediate greater siRNA delivery to activated fibroblasts than both the benchmark MC3 LNP and non-targeted T3A-C12 LNP. Moreover, AA-T3A-C12 LNP-mediated enhanced transfection of activated fibroblasts was further confirmed by luciferase mRNA delivery (FIGS. 18A-18B), as AA-T3A-C12 LNP achieved 2.6-fold higher luciferase expression in fibroblasts compared to MC3 LNP, but 1.4-fold lower in hepatocytes. This result indicates AA-T3A-C12 LNP hold the potential for targeted delivery of large genetic constructs to activated fibroblasts as well.

### Example 5: AA-T3A-C12 LNP-Mediated Robust Gene Knockdown in Activated Fibroblasts

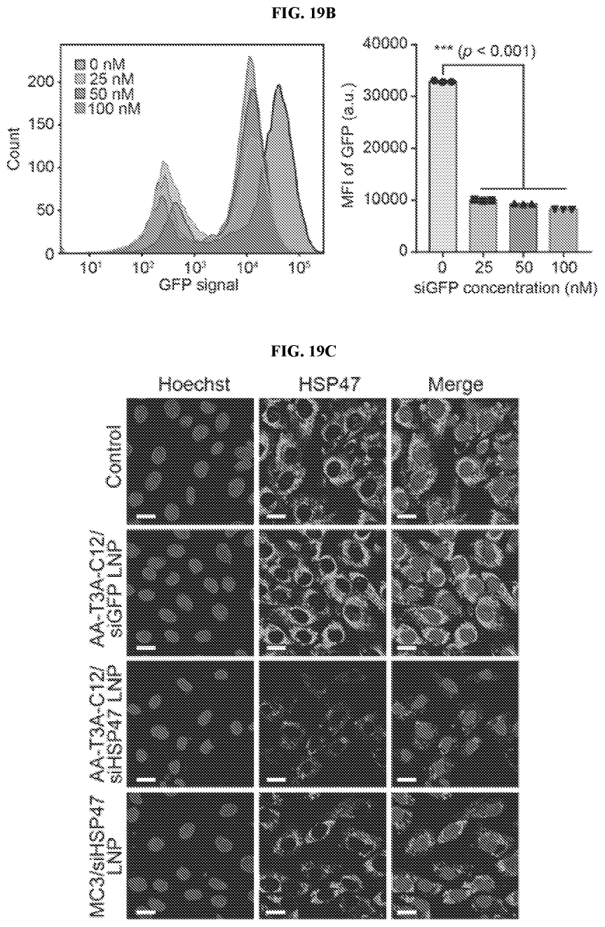

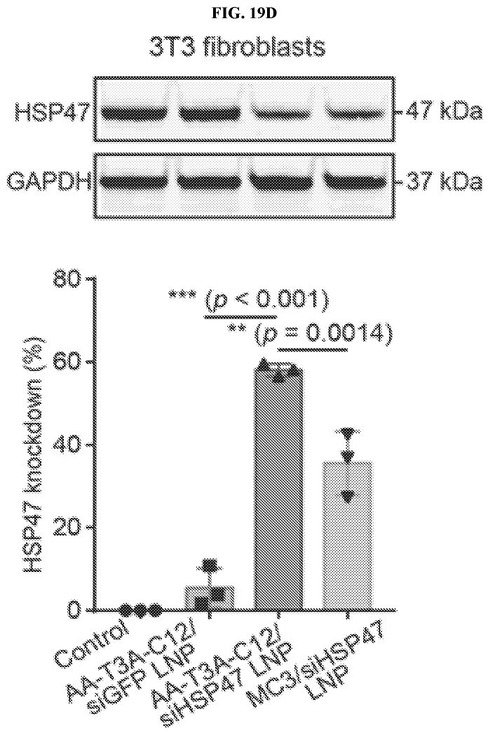

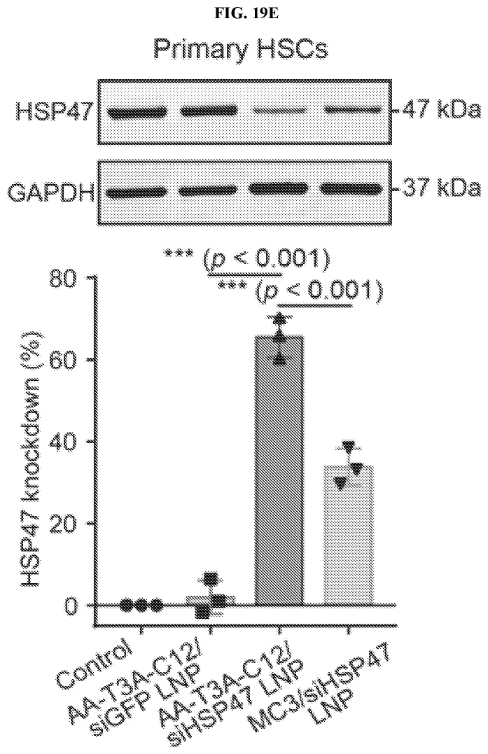

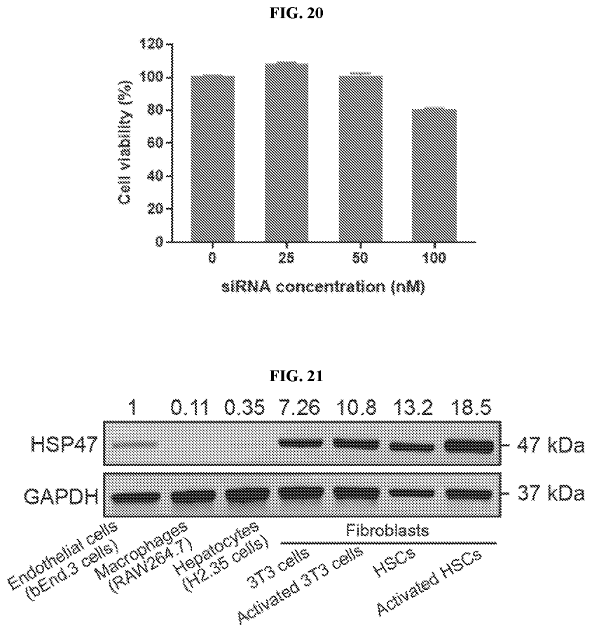

After confirming AA-T3A-C12 LNP-mediated targeted gene delivery to activated fibroblasts, its potency for gene silencing was then investigated. In activated 3T3-GFP fibroblasts, AA-T3A-C12/siGFP LNP achieved time- and dose-dependent GFP knockdown in a highly efficient manner (FIG. 19A). After treatment with 50 nM of AA-T3A-C12/siGFP LNP for 48 h, >80% GFP silencing was achieved without noticeable cytotoxicity (FIG. 20), which is in agreement with the high-throughput screening results described elsewhere herein (FIG. 7B and FIG. 9). The GFP silencing effect of this LNP was further confirmed by flow cytometry analysis of GFP expression, with potent GFP knockdown achieved at an siGFP dose as low as 25 nM (FIG. 19B).

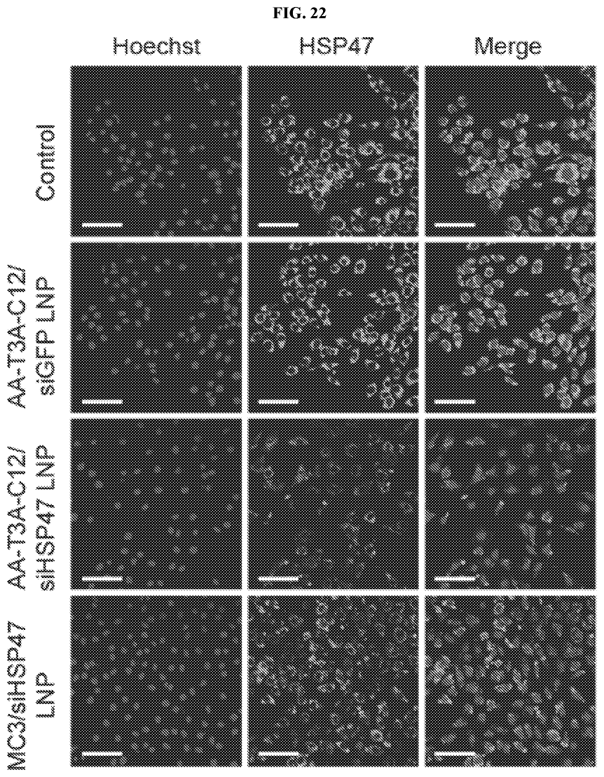

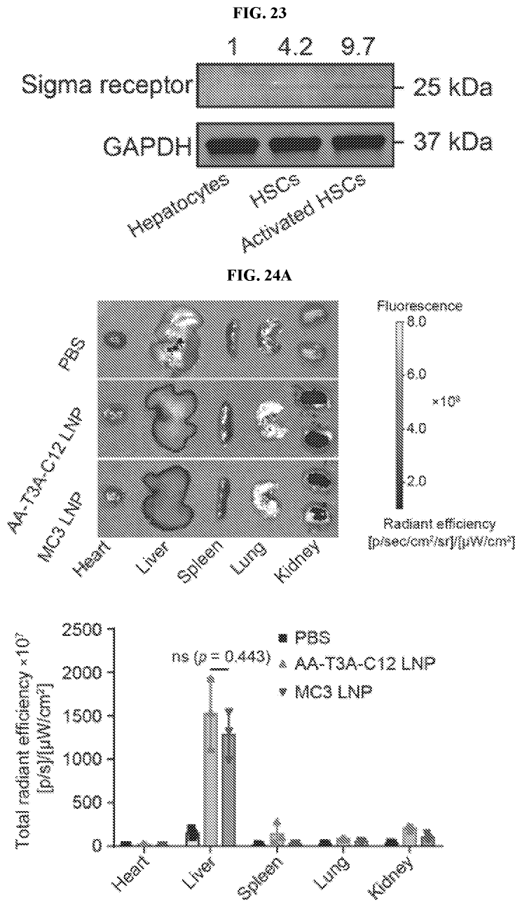

The potential of siHSP47-loaded AA-T3A-C12 to silence HSP47, a therapeutic target of liver fibrosis, was next evaluated, and subsequently compared with the benchmark MC3 LNP. Notably, activated fibroblasts/HSCs were identified as the primary source of HSP47 in the fibrotic liver, which was further confirmed (FIG. 21). Immunofluorescence (IF) staining results showed that HSP47 expression in activated 3T3 fibroblasts was largely inhibited after AA-T3A-Cl₂/siHSP47 LNP treatment (FIG. 19C and FIG. 22), and its effect was more potent than MC3/siHSP47 LNP. In contrast, AA-T3A-C12/siGFP LNP had no effect on the expression of HSP47. The superior silencing ability of AA-T3A-C12/siHSP47 LNP in activated 3T3 fibroblasts was further confirmed by Western blot analysis (FIG. 19D), as it mediated approximately 60% down-regulation of HSP47, which was much higher than the approximately 35% knockdown achieved by MC3/siHSP47 LNP. Afterwards, their silencing activities were evaluated in activated primary HSCs, which were confirmed to express a much higher level of sigma receptors than hepatocytes (FIG. 23). The results showed that AA-T3A-C12/siHSP47 LNP achieved significantly higher knockdown efficiency compared to MC3/siHSP47 LNP (65% vs 34%, FIG. 19E). Together, these results confirm that targeted delivery of siRNA into activated fibroblasts using AA-T3A-C12 LNP enables robust gene knockdown.

### Example 6: Potent In Vivo HSP47 Silencing Via AA-T3A-C12 LNP

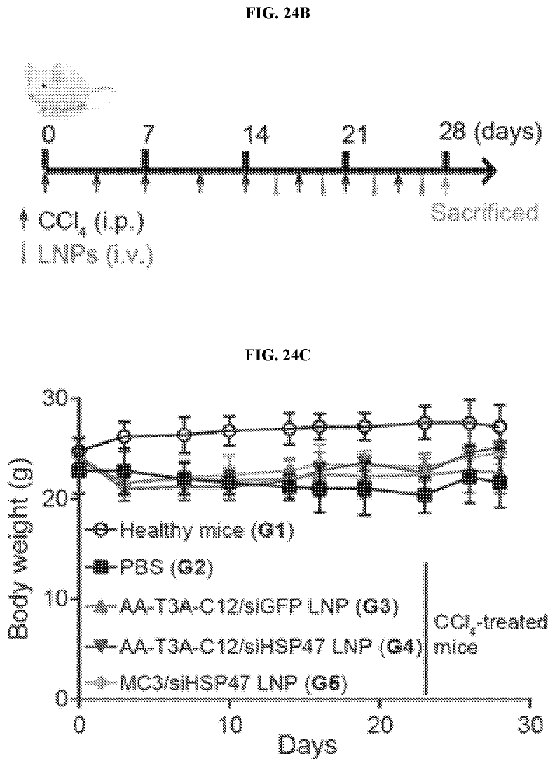

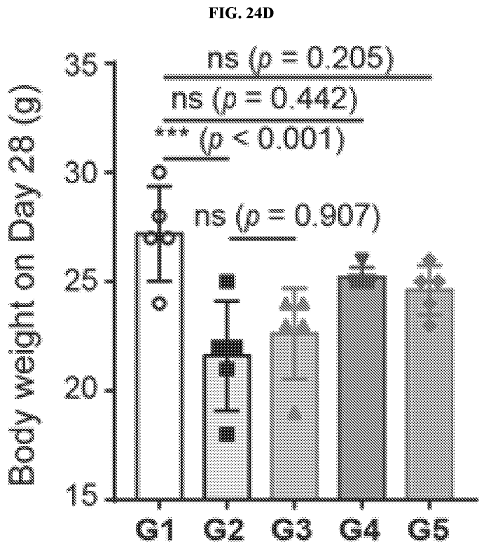

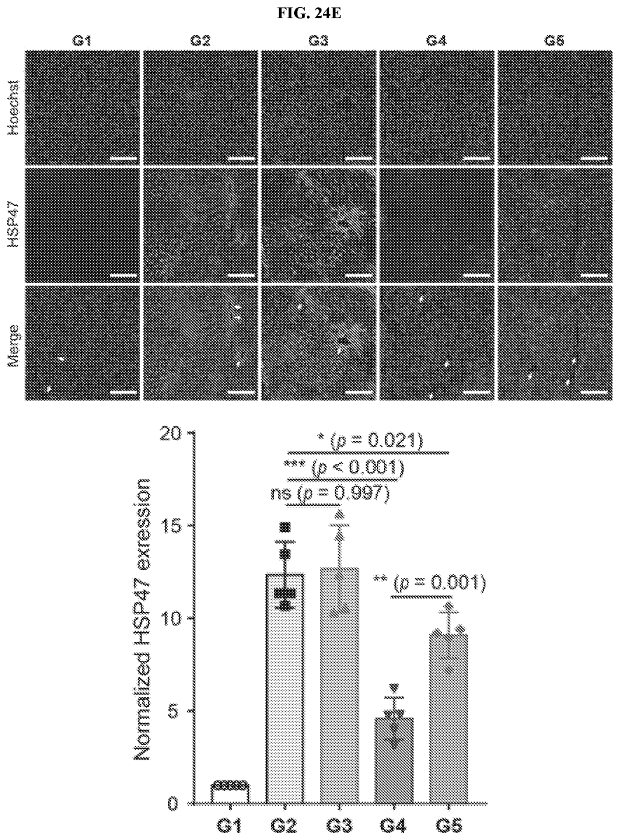

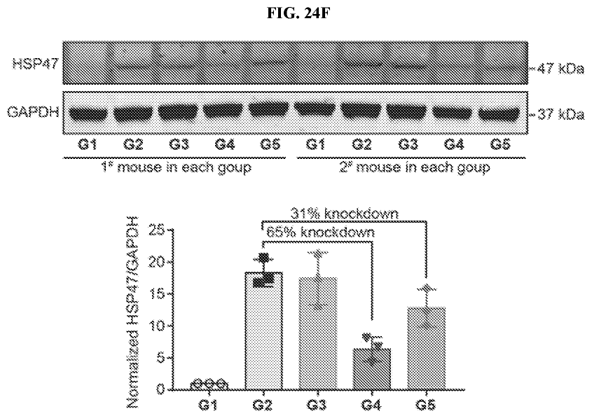

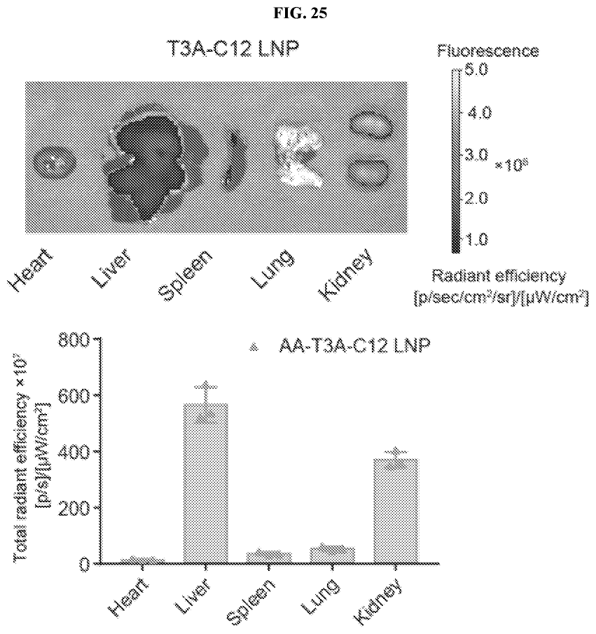

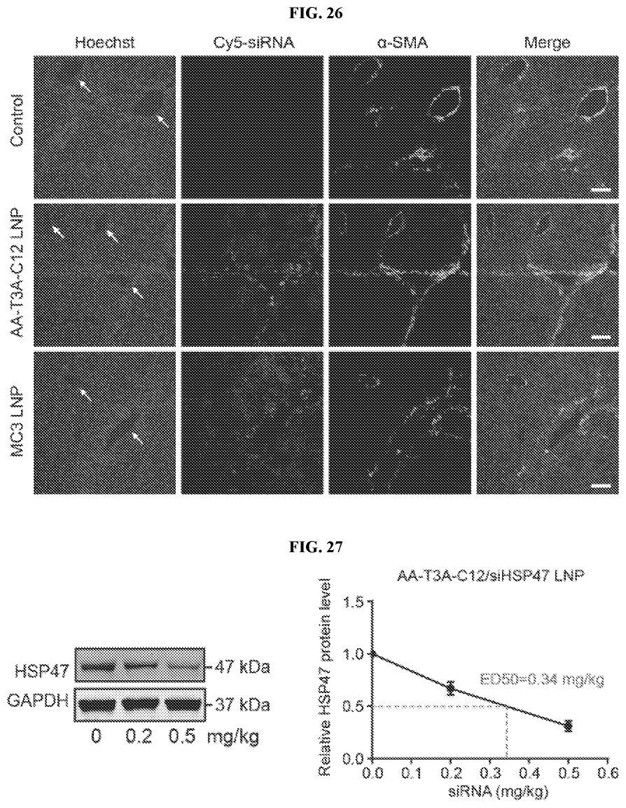

After confirming potent in vitro HSP47 silencing, the in vivo performance and therapeutic potential of AA-T3A-C12/siHSP47 LNP was evaluated. First, the biodistribution was investigated in fibrotic mice after tail vein injection of Cy5-siRNA-loaded LNPs. MC3 LNP was included as a positive control due to its well-known hepatic accumulation and transfection. As expected, MC3 LNP predominantly accumulated in the liver (FIG. 24A). AA-T3A-C12 LNP exhibited a similar organ distribution pattern as MC3 LNP, with primary liver localization. Since there were no observable differences in liver accumulation between AA-T3A-C12 LNP and MC3 LNP (p=0.443), they were compared for in vivo silencing activity and therapeutic efficacy. Non-targeted T3A-C12 LNP was not included for further studies due to its low siRNA EE and poor liver accumulation (Table 3 and FIG. 25). To further investigate the HSC-targeting ability of LNPs, livers were cryosectioned and α-smooth muscle actin (i.e., α-SMA, a marker of activated HSCs) was stained for immunofluorescence analysis. Confocal imaging results showed that more AA-T3A-C12 LNP co-localized with or were close to activated HSCs than was observed with MC3 LNP (FIG. 26), presumably due to the strong affinity between anisamide ligands and sigma receptors. Together, these results demonstrate the superiority of AA-T3A-C12 LNP to target the liver and activated HSCs.

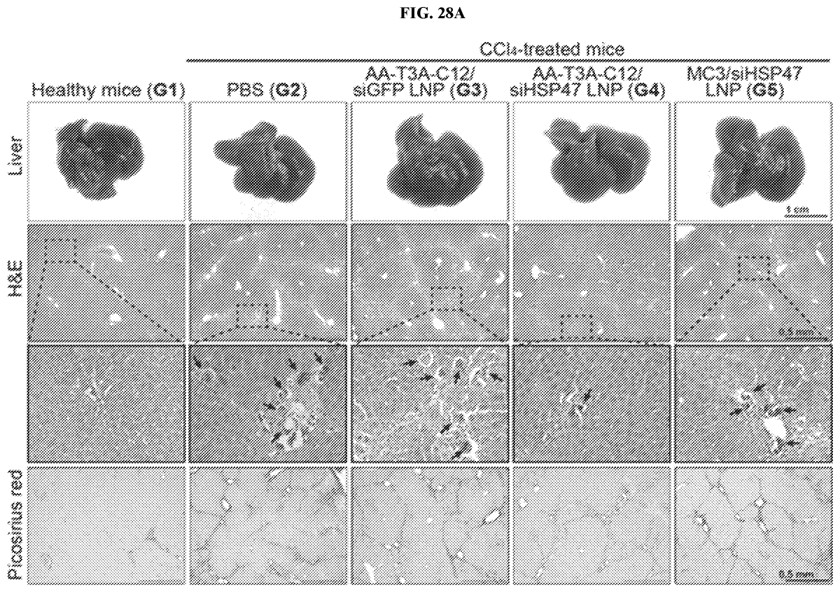

Next, therapeutic studies were conducted in fibrotic mice that were treated with CCl₄ twice weekly for 4 weeks to induce liver fibrosis (FIG. 24B). Healthy mice were used as an untreated control (i.e., Group 1 or G1). During the last two weeks of the experiment, fibrotic mice were treated twice weekly with PBS (G2), AA-T3A-C12/siGFP LNP (G3), AA-T3A-C12/siHSP47 LNP (G4) or MC3/siHSP47 LNP (G5). The siRNA dose was pre-determined to be μg (0.2 mg/kg) for each injection, which was lower than the median effective dose (ED₅₀=0.34 mg/kg) for AA-T3A-C12/siHSP47 LNP but still achieved moderate HSP47 knockdown (FIG. 27). Interestingly, although all CCl₄-treated mice suffered substantial weight loss compared to healthy mice at the beginning, fibrotic mice treated with AA-T3A-C12/siHSP47 LNP or MC3/siHSP47 LNP gradually gained body weight (FIG. 24C). At the end of experiment, the body weight of AA-T3A-C12/siHSP47 LNP- or MC3/siHSP47 LNP-treated fibrotic mice was comparable to healthy mice (FIG. 24D). Additionally, AA-T3A-C12/siGFP LNP treatment did not cause additional weight loss compared to PBS treatment in fibrotic mice (p=0.907). These results suggest that AA-T3A-C12 LNP is well-tolerated by mice and that silencing of HSP47 (FIGS. 24E-24F) could be helpful to overcome liver fibrosis-mediated weight loss.

To observe the changes of HSP47 expression, liver samples from each group were sectioned for IF staining. Confocal imaging of liver sections demonstrated that minimal HSP47 expression was observed in healthy mice, but CCl₄ treatment induced abundant expression of HSP47 especially around central veins (FIG. 24E). Encouragingly, AA-T3A-C12/siHSP47 LNP treatment significantly down-regulated HSP47 expression in fibrotic mice. In contrast, MC3/siHSP47 LNP treatment only moderately reduced HSP47 expression due to its predominant hepatocyte transfection. Western blot analysis of HSP47 in liver lysates showed that minimal HSP47 was detected in healthy mice (FIG. 24F), but a significant increase of HSP47 was observed in fibrotic mice. However, HSP47 expression in fibrotic mice was largely suppressed by AA-T3A-C12/siHSP47 LNP treatment. Quantitative analysis of Western blot results indicated that AA-T3A-C12/siHSP47 LNP treatment led to 65% knockdown of HSP47 compared to PBS treatment in fibrotic mice, which was >2-fold more potent than 31% knockdown achieved by MC3/siHSP47 LNP. Together, these results demonstrate that potent HSP47 silencing can be achieved in fibrotic mice by AA-T3A-C12/siHSP47 LNP.

### Example 7: Reduction of Liver Fibrosis after AA-T3A-C12 LNP-Mediated HSP47 Silencing

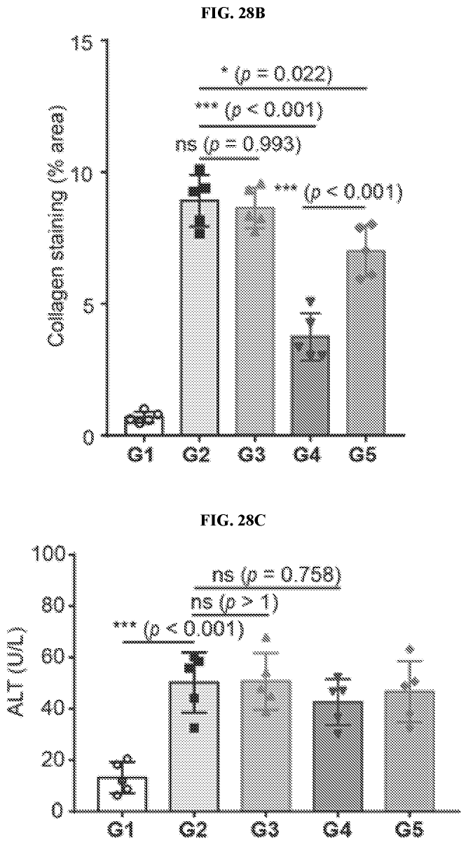

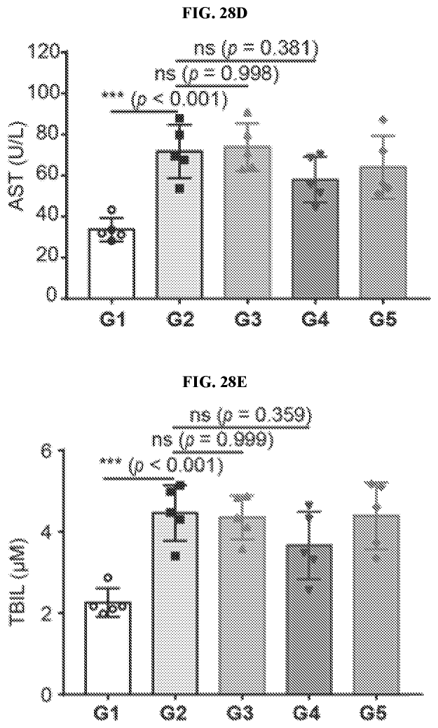

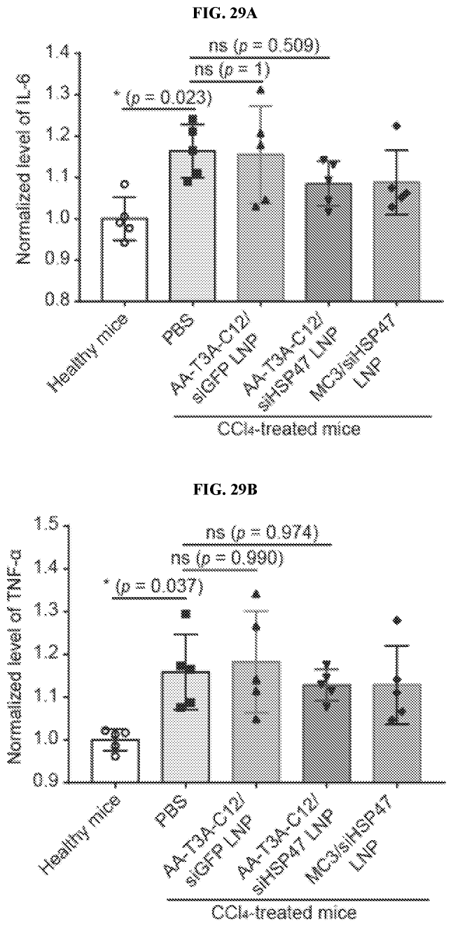

After confirming the superior knockdown properties of HSP47 by AA-T3A-C12/siHSP47 LNP, its anti-fibrotic efficacy was examined. Livers harvested from different treatment groups were subjected to macroscopic and histopathological analysis. Livers from healthy mice appeared to have a regular and smooth surface, while livers from fibrotic mice had a rough surface due to CCl₄-induced liver damage (FIG. 28A). Encouragingly, treatment with AA-T3A-C12/siHSP47 LNP enhanced the recovery of the damaged liver, as it had a more smooth and normal appearance than other treatment groups. Hematoxylin and eosin (H&E) staining of healthy livers revealed normal histological architecture. CCl₄ treatment induced extensive neutrophil infiltration and irregular regenerating pseudolobules with dense fibrotic septa in the liver. Additionally, extensive contraction and death of hepatocytes was observed in CCl₄-treated mice.

However, livers from AA-T3A-C12/siHSP47 LNP-treated fibrotic mice exhibited a more normal histological structure and had less fibrotic septa, as well as less apoptotic hepatocytes compared to other treatment groups. Liver sections were further stained with Picosirius red to evaluate collagen coverage and tissue fibrosis. Healthy livers revealed normal lobular architecture and minimal collagen fibers. As expected, CCl₄ treatment led to extensive collagen deposition and pseudolobular formation, but AA-T3A-C12/siHSP47 LNP treatment remarkably reduced collagen fibers and pseudolobules with minimal collagen staining around central veins. Morphometric quantification of Picosirius red stained areas further confirmed the significantly decreased collagen deposition in AA-T3A-C12/siHSP47 LNP-treated fibrotic mice, which was more significant than that of MC3/siHSP47 LNP-treated group (p<0.001, FIG. 28B). Together, these results suggest that targeted knockdown of HSP47 in activated HSCs enabled by AA-T3A-C12 LNP successfully reduce collagen deposition and alleviate liver fibrosis, with better anti-fibrotic efficacy than MC3 LNP.

### Example 8: Safety Evaluation

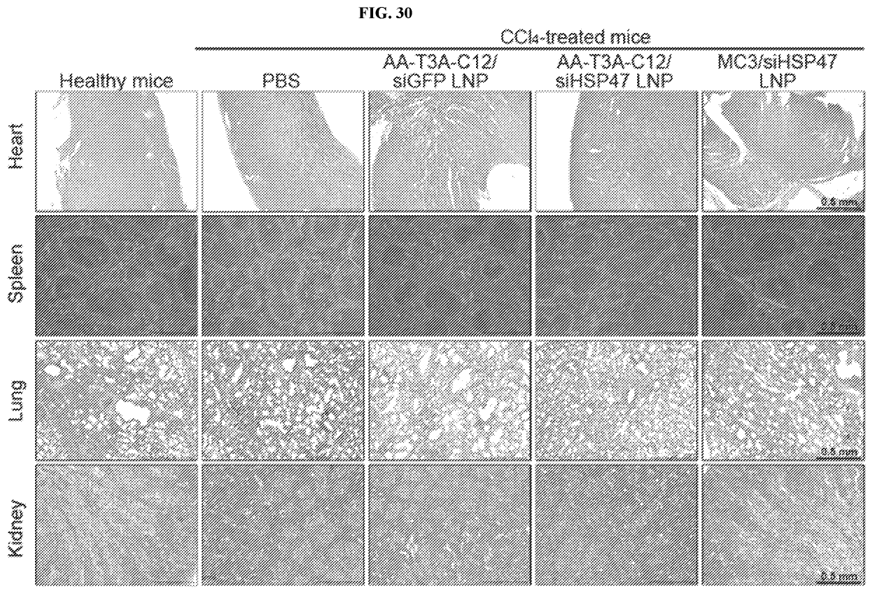

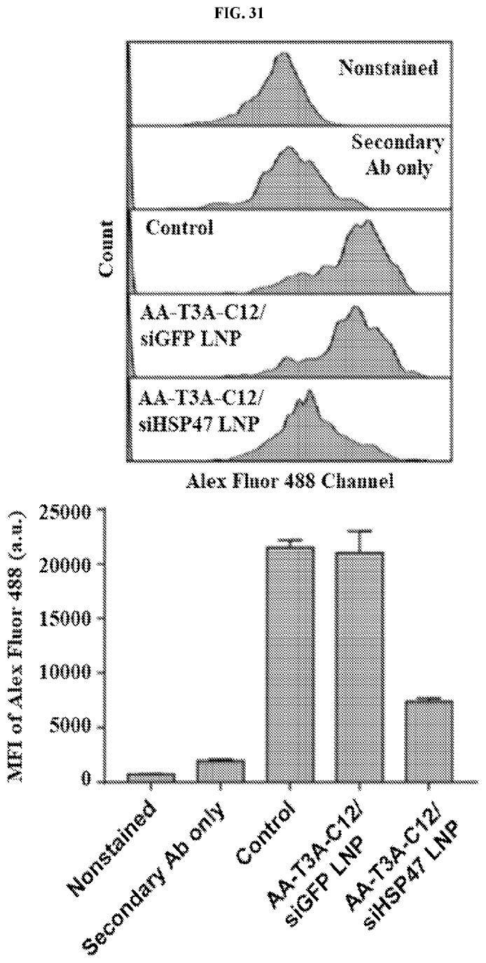

Safety evaluation of these treatment regimens were conducted. First, serum alanine aminotransferase (ALT), aspartate aminotransferase (AST) and total bilirubin (TBIL) were quantified to assess liver toxicity (FIG. 28C-28E). ALT, AST and TBIL markedly increased in fibrotic mice compared to healthy mice, as a result of CCl₄-induced liver injury. AA-T3A-C12/siGFP LNP treatment did not further increase ALT, AST and TBIL levels, indicating that AA-T3A-C12 LNP was well-tolerated without exacerbating liver injury. Moreover, AA-T3A-C12/siHSP47 LNP treatment slightly decreased ALT, AST and TBIL levels in fibrotic mice, but these decreases were not significant. Since fibrotic mice were continuously insulted with CCl₄ (FIG. 24B), it is reasonable to expect that liver functions were not significantly improved despite the obvious attenuation of the fibrotic process after AA-T3A-C12/siHSP47 LNP treatment. Additionally, although CCl₄ treatment resulted in the elevation of proinflammatory cytokines including tumor necrosis factor (TNF)-α and interleukin (IL)-6 (FIGS. 29A-29B), all LNP treatment regimens did not increase inflammation levels. Further, histopathological analysis of other organs including heart, spleen, lung and kidney were performed (FIG. 30). No histological differences were observed in these organs in all fibrotic mice groups compared to the healthy mice group, suggesting that neither CCl₄ nor LNPs induced noticeable damage to these organs. Together, these results demonstrate a good safety profile of AA-T3A-C12/siHSP47 LNP.

### ENUMERATED EMBODIMENTS

The following exemplary embodiments are provided, the numbering of which is not to be construed as designating levels of importance:

Embodiment 1 provides a compound of Formula (I), or a salt, solvate, stereoisomer, tautomer, or isotopologue thereof:

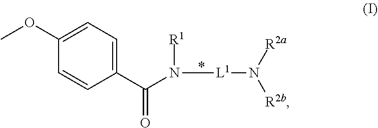

wherein:
L¹ is selected from the group consisting of -(optionally substituted C₂-C₁₂ heteroalkylenyl)- and -(optionally substituted C₂-C₁₂ heteroalkylenyl)-(optionally substituted C₂-C₁₂ heterocycloalkylenyl)-,
   wherein each occurrence of C₂-C₁₂ heteroalkylenyl and C₂-C₁₂ heterocycloalkylenyl is optionally substituted with at least one substituent selected from the group consisting of optionally substituted C₁-C₂₄ alkyl, optionally substituted C₁-C₂₄ heteroalkyl optionally substituted C₃-C₈ cycloalkyl, optionally substituted C₆-C₁₀ aryl, optionally substituted C₂-C₁₀ heteroaryl, R^{2c}, and

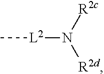

or two geminal substituents may combine to form ═O;
each occurrence of L² is independently selected from the group consisting of -(optionally substituted C₁-C₆ alkylenyl)-, -(optionally substituted C₂-C₆ heteroalkylenyl)-, -(optionally substituted C₁-C₆ alkylenyl)-C(═O)X-(optionally substituted C₁-C₆ alkylenyl)-, and -(optionally substituted C₁-C₆ alkylenyl)-XC(═O)-(optionally substituted C₁-C₆ alkylenyl)-;
R¹ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{2a}, R^{2b}, R^{2c}, and R^{2d}, if present, is independently selected from the group consisting of H, -(optionally substituted C₁-C₆ alkylenyl)-C(═O)OR³, -(optionally substituted C₁-C₆ alkylenyl)-C(═O)N(R³)(R⁴), -(optionally substituted C₁-C₆ alkylenyl)-C(═O)R³, -(optionally substituted C₁-C₆ alkylenyl)-(R³), —C(═O)OR³, —C(═O)N(R³)(R⁴), —C(═O)R³, and R³,
   wherein no more than one of each occurrence of R^{2a}, R^{2b}, R^{2c}, and R^{2d} is H;
R³ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁴ is selected from the group consisting of H and optionally substituted C₁-C₆ alkyl;
each occurrence of X is independently selected from the group consisting of a bond, 0, and NR⁵; and
each occurrence of R⁵ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

Embodiment 2 provides the compound of Embodiment 1, wherein R¹ is H.

Embodiment 3 provides the compound of Embodiment 1 or 2, wherein L¹ is selected from the group consisting of:

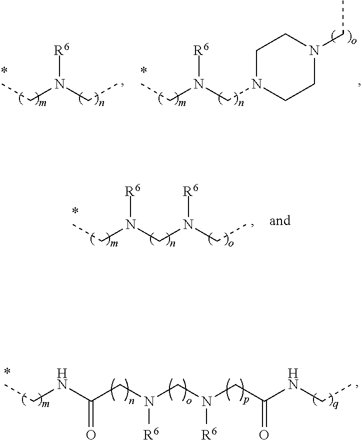

wherein:
each occurrence of R⁶ is independently selected from the group consisting of H, optionally substituted C₁-C₂₄ alkyl, and

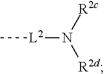

m, n, o, p, and q are each independently an integer selected from the group consisting of 1, 2, 3, and 4.

Embodiment 4 provides the compound of Embodiment 3, wherein R⁶ is selected from the group consisting of H, CH₃, CH₂CH(OH)(optionally substituted C₁-C₁₂ alkyl) and

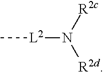

Embodiment 5 provides the compound of any one of Embodiments 1-4, wherein L² is selected from the group consisting of —CH₂CH₂—, —CH₂CH₂CH₂—, and —CH₂CH₂—C(═O)NH—CH₂CH₂—.

Embodiment 6 provides the compound of Embodiment 1 wherein L¹ is selected from the group consisting of

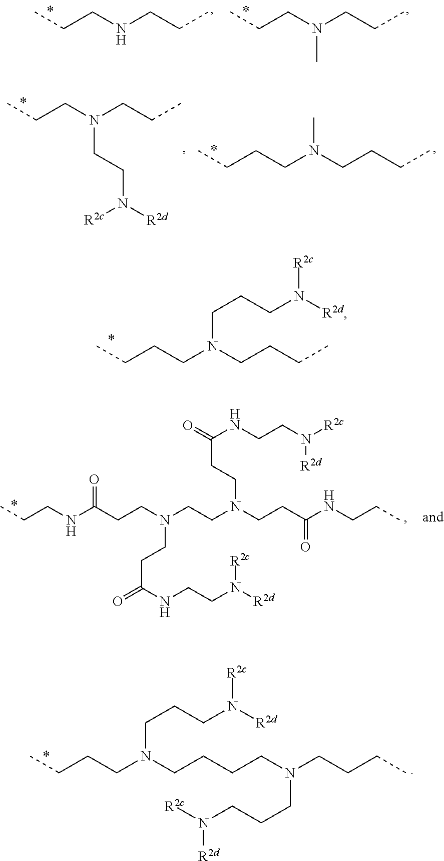

Embodiment 7 provides the compound of any one of Embodiments 1-6, wherein R^{2a}, R^{2b}, R^{2c}, and R^{2d}, if present, are each independently selected from the group consisting of H, —CH₂CH(OH)(optionally substituted C₁-C₂₈ alkyl), —CH₂CH(OH)(optionally substituted C₂-C₂₈ alkenyl), —CH₂CH₂C(═O)O(optionally substituted C₁-C₂₈ alkyl), and —CH₂CH₂C(═O)NH(optionally substituted C₁-C₂₈ alkyl).

Embodiment 8 provides the compound of any one of Embodiments 1-7, wherein R^{2a}, R^{2b}, R^{2c}, and R^{2d}, if present, are each independently selected from the group consisting of —CH₂CH(OH)(CH₂)₉CH₃, —CH₂CH(OH)(CH₂)₁₁CH₃, and —CH₂CH(OH)(CH₂)₁₃CH₃.

Embodiment 9 provides the compound of any one of Embodiments 1-8, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, and optionally substituted heteroalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 10 provides the compound of any one of Embodiments 1-9, which is selected from the group consisting of:

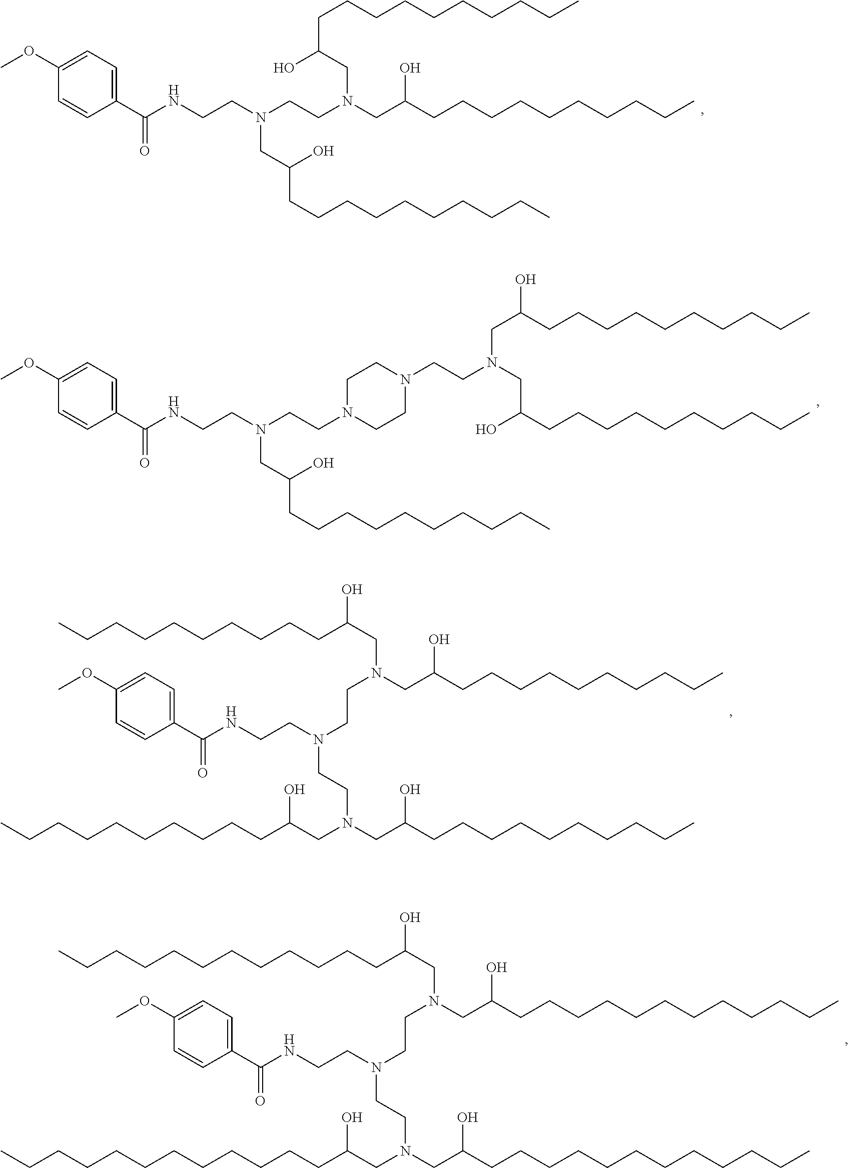

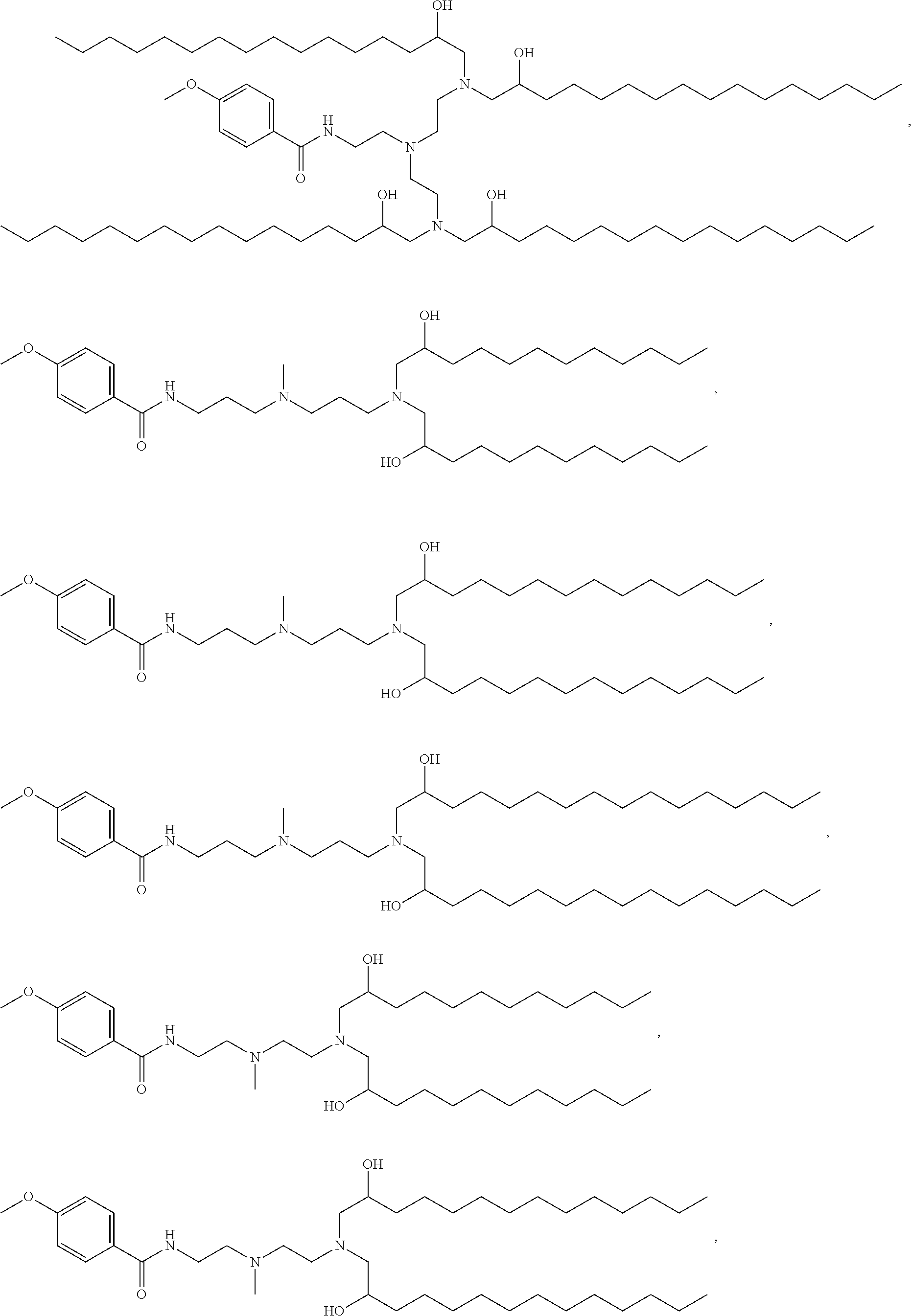

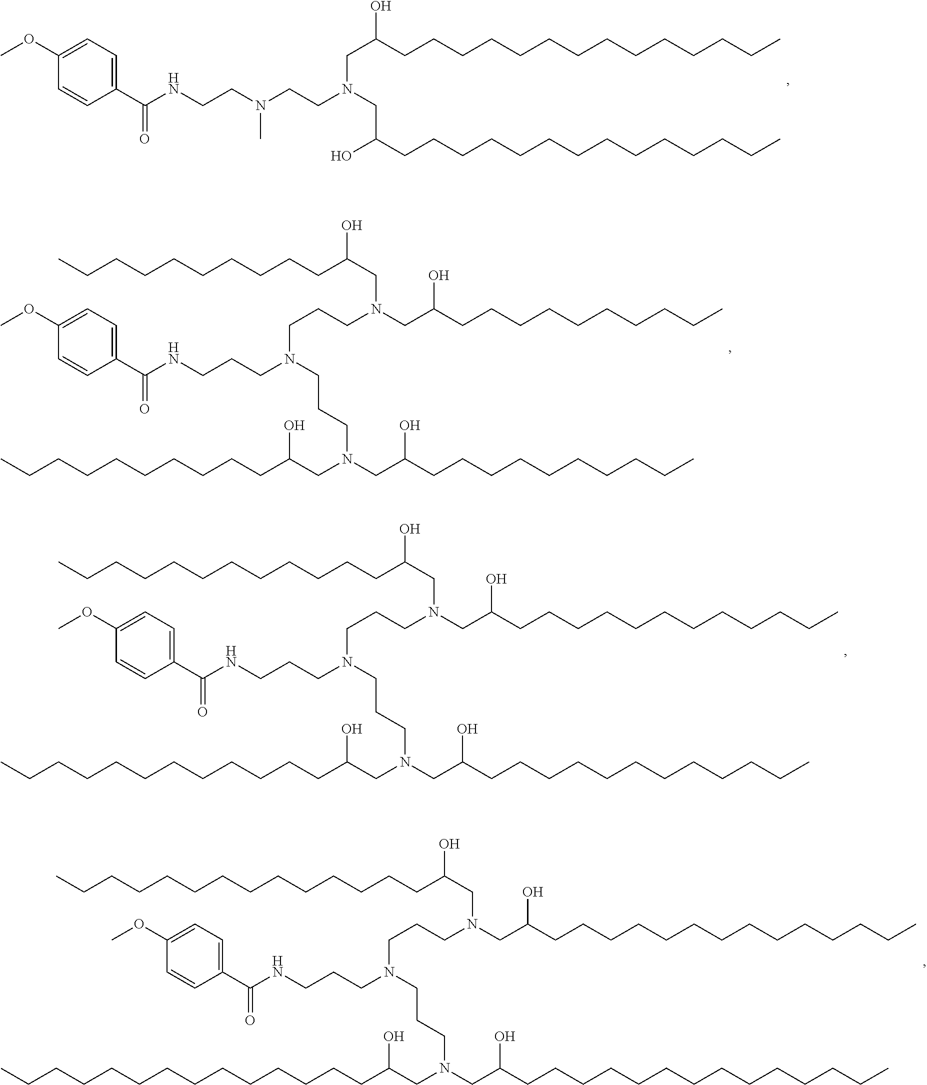

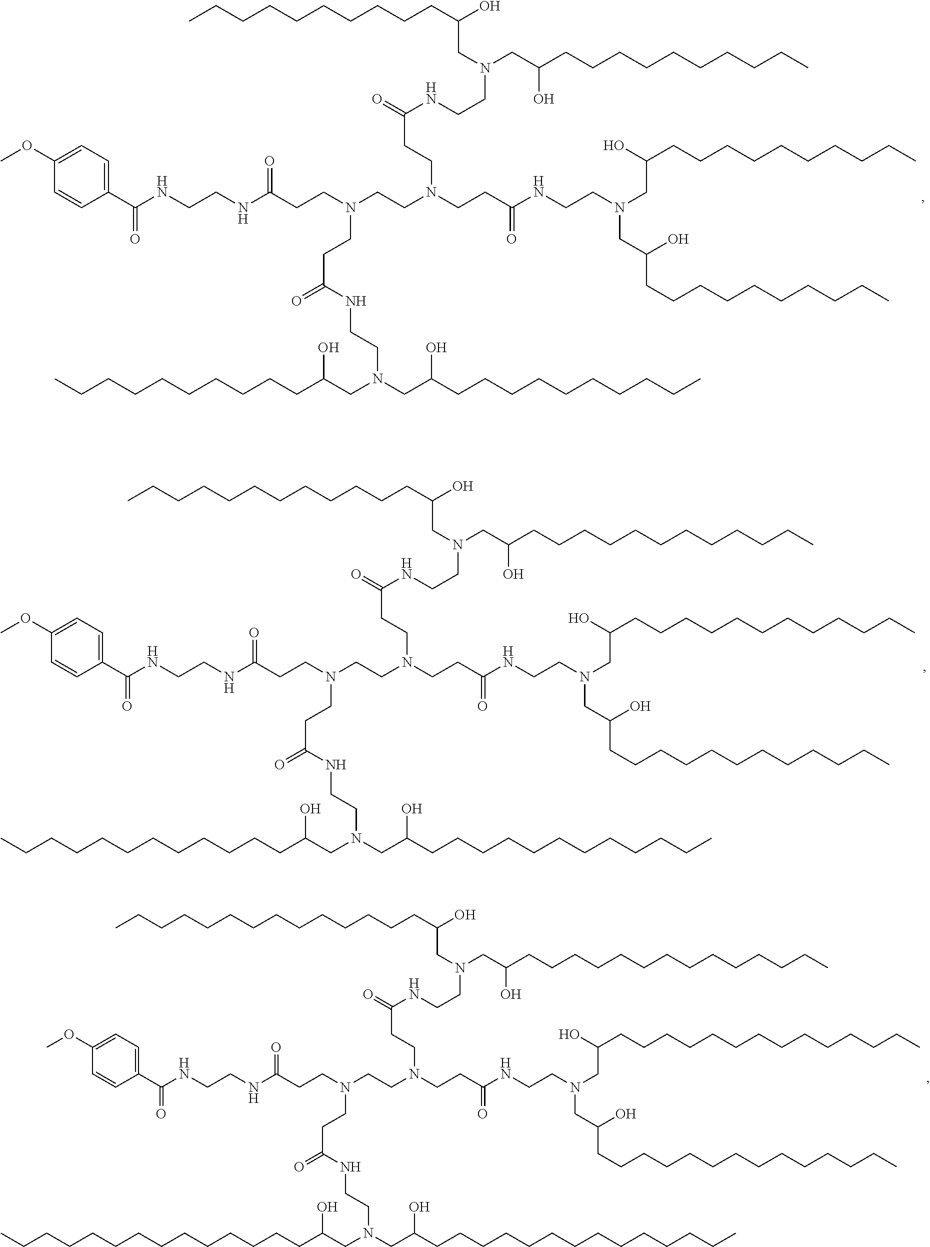

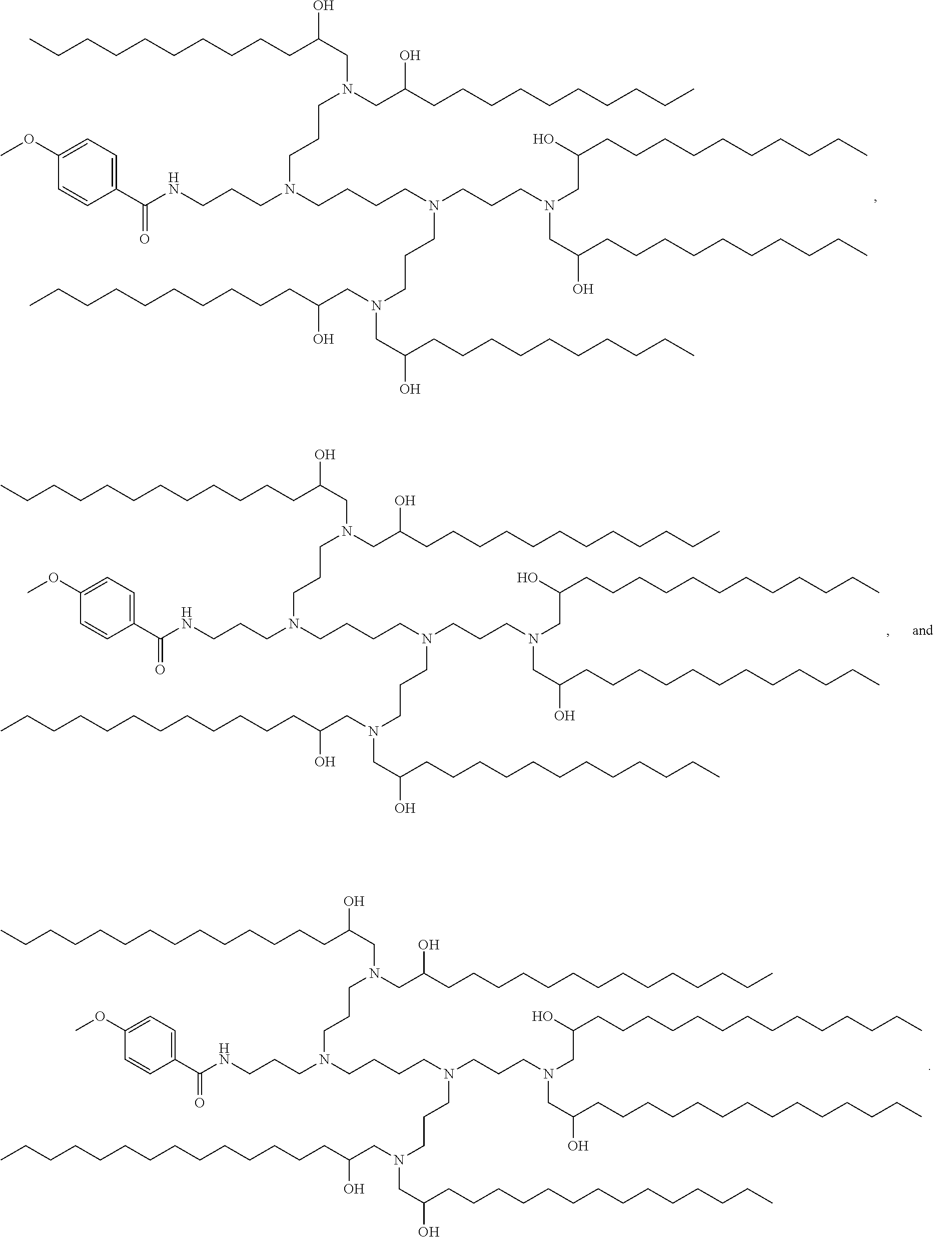

Embodiment 11 provides a lipid nanoparticle (LNP) comprising:
(a) at least one compound of any one of Embodiments 1-10;
(b) at least one helper lipid;
(c) at least one cholesterol lipid; and
(d) at least one conjugated lipid.

Embodiment 12 provides the LNP of Embodiment 11, wherein R¹ is H.

Embodiment 13 provides the LNP of Embodiment 11 or 12, wherein L¹ is selected from the group consisting of:

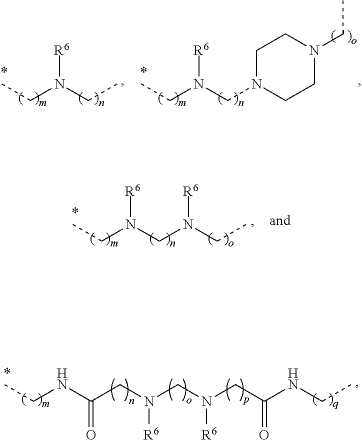

wherein:
each occurrence of R⁶ is independently selected from the group consisting of H, optionally substituted C₁-C₂₄ alkyl, and

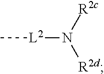

m, n, o, p, and q are each independently an integer selected from the group consisting of 1, 2, 3, and 4.

Embodiment 14 provides the LNP of Embodiment 13, wherein R⁶ is selected from the group consisting of H, CH₃, CH₂CH(OH)(optionally substituted C₁-C₁₂ alkyl) and

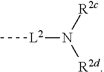

Embodiment 15 provides the LNP of any one of Embodiments 11-14, wherein L² is selected from the group consisting of —CH₂CH₂—, —CH₂CH₂CH₂—, and —CH₂CH₂—C(═O)NH—CH₂CH₂—.

Embodiment 16 provides the LNP of any one of Embodiments 11-15, wherein L¹ is selected from the group consisting of

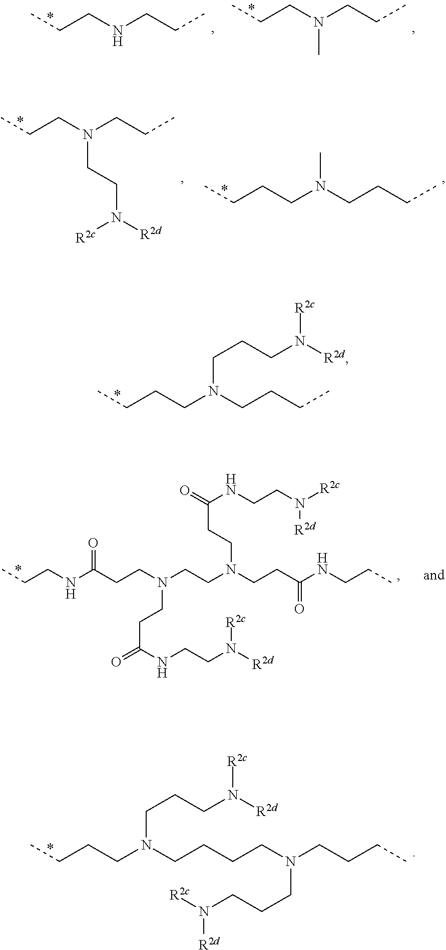

Embodiment 17 provides the LNP of any one of Embodiments 11-16, wherein R^{2a}, R^{2b}, R^{2c}, and R^{2d}, if present, are each independently selected from the group consisting of H, —CH₂CH(OH)(optionally substituted C₁-C₂₈ alkyl), —CH₂CH(OH)(optionally substituted C₂-C₂₈ alkenyl), —CH₂CH₂C(═O)O(optionally substituted C₁-C₂₈ alkyl), and —CH₂CH₂C(═O)NH(optionally substituted C₁-C₂₈ alkyl).

Embodiment 18 provides the LNP of any one of Embodiments 11-17, wherein R^{2a}, R^{2b}, R^{2c}, and R^{2d}, if present, are each independently selected from the group consisting of —CH₂CH(OH)(CH₂)₉CH₃, —CH₂CH(OH)(CH₂)₁₁CH₃, and —CH₂CH(OH)(CH₂)₁₃CH₃.

Embodiment 19 provides the LNP of any one of Embodiments 11-18, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, and optionally substituted heteroalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R¹, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 20 provides the LNP of any one of Embodiments 11-19, which is selected from the group consisting of:

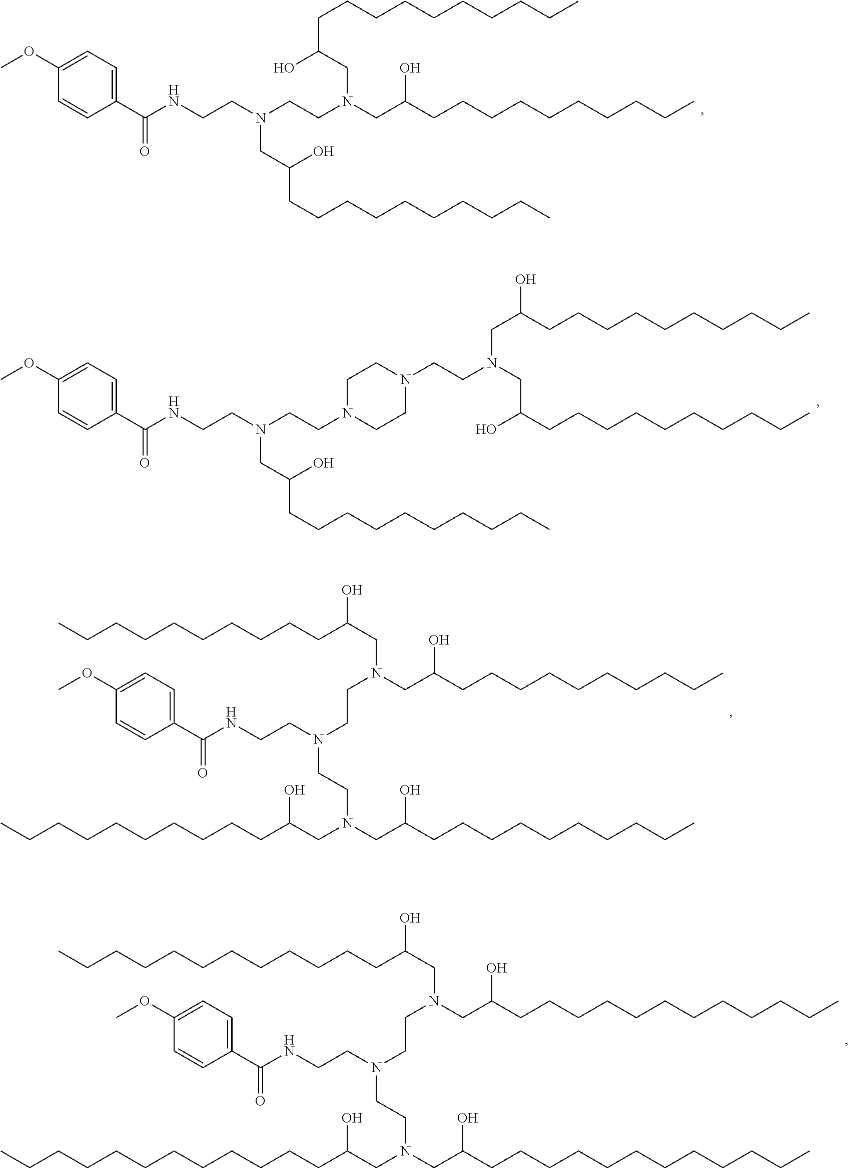

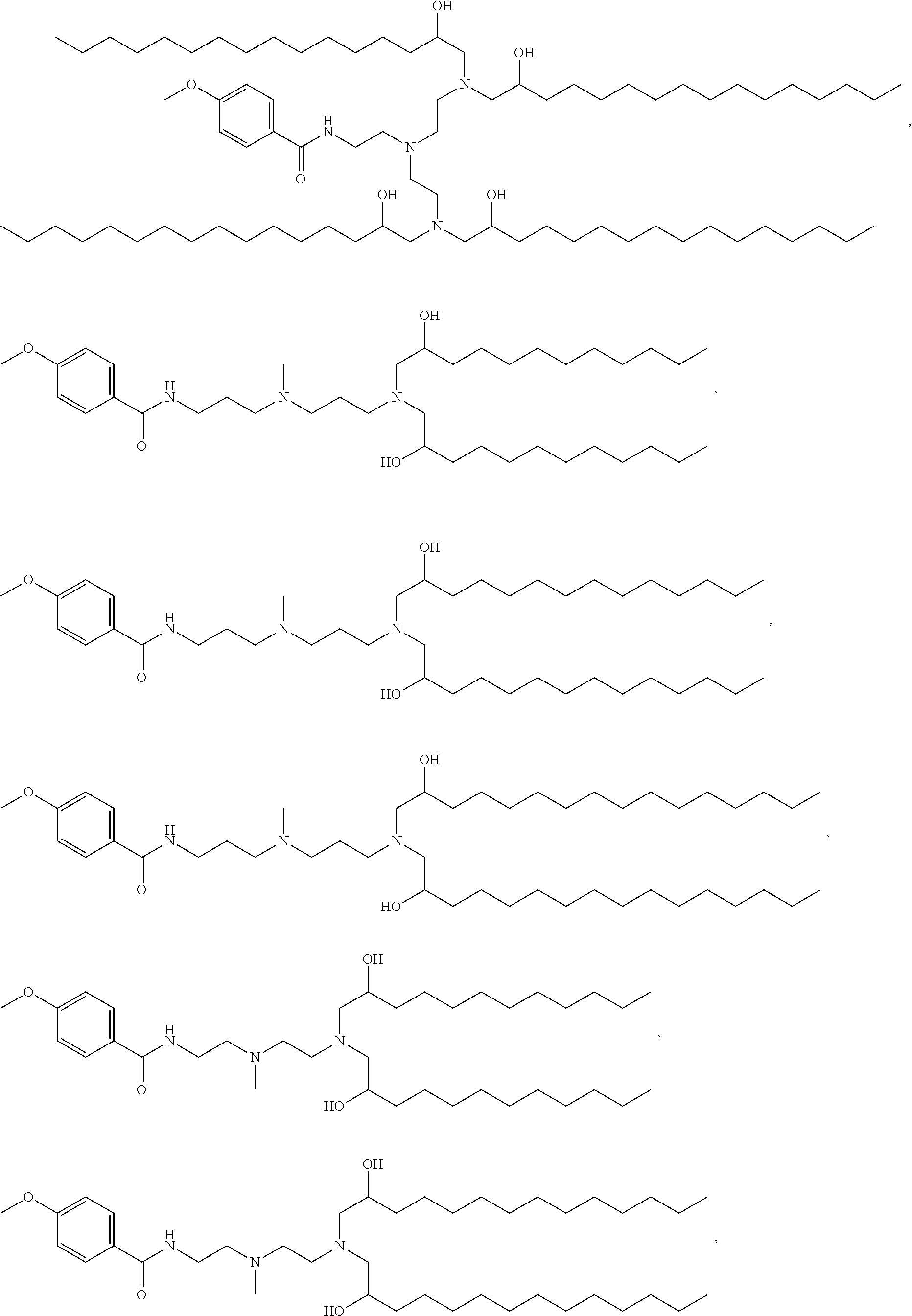

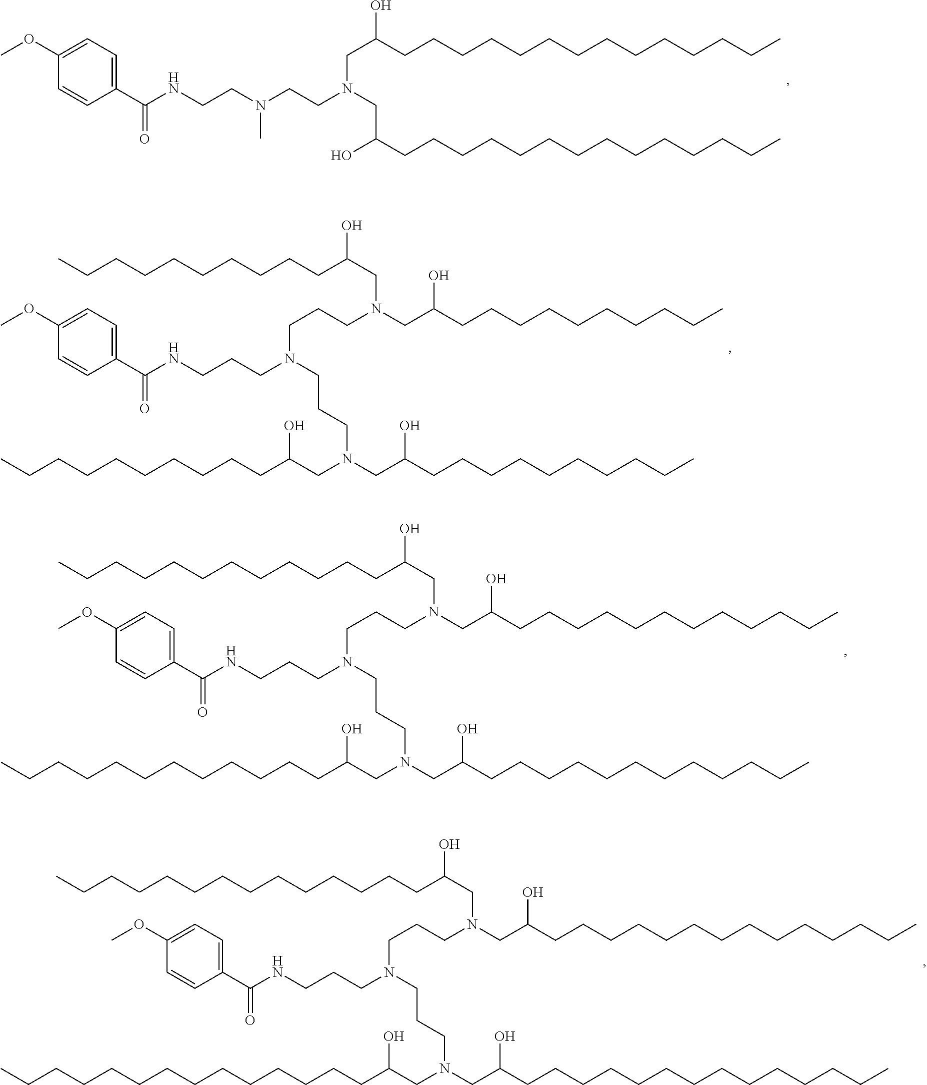

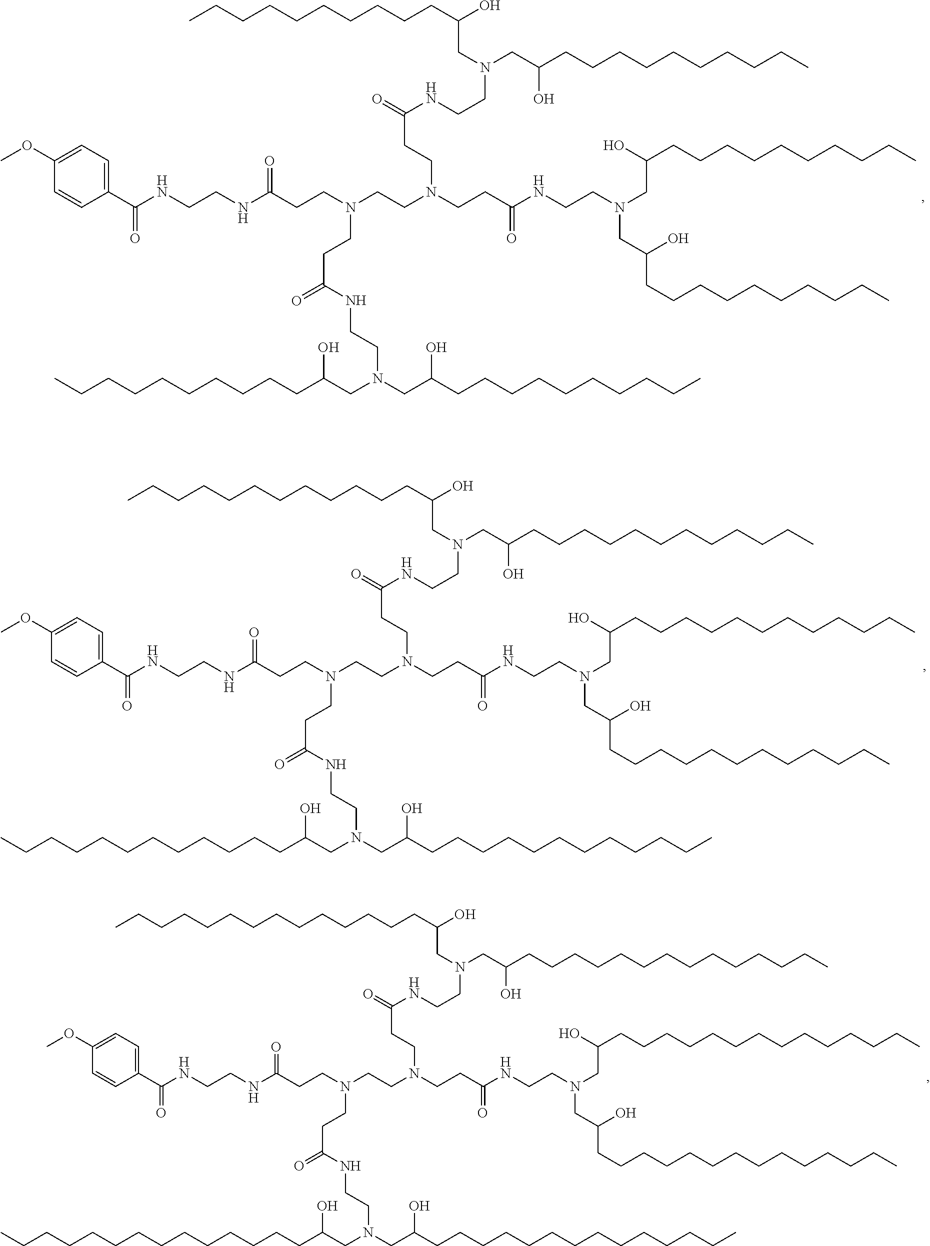

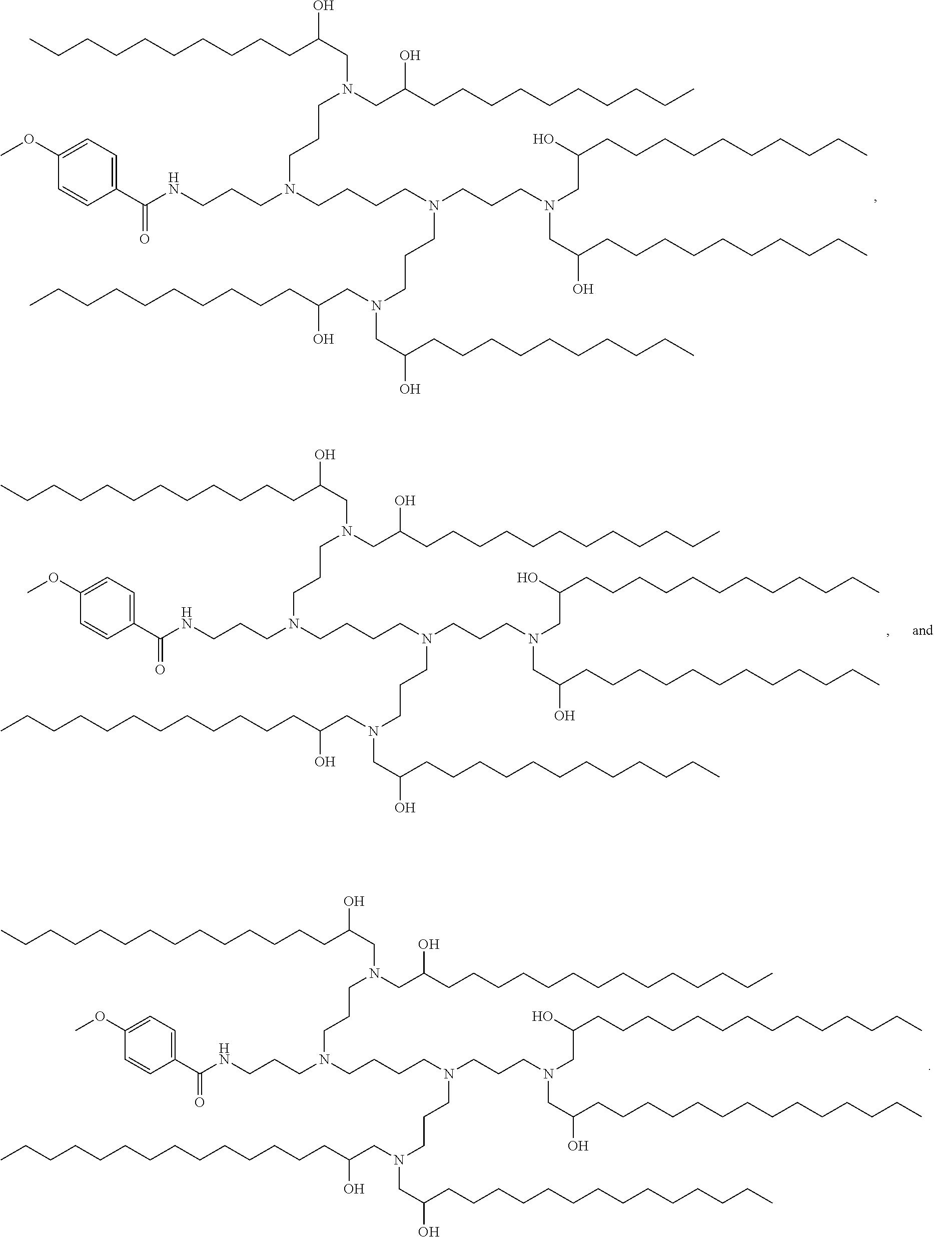

Embodiment 21 provides the LNP of any one of Embodiments 11-20, wherein the compound is:

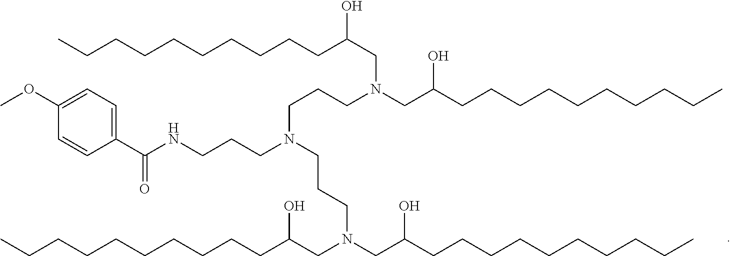 [SMILES: CCCCCCCCCCC(O)CN(CCCN(CCCNC(=O)c1ccc(OC)cc1)CCCN(CC(O)CCCCCCCCCC)CC(O)CCCCCCCCCC)CC(O)CCCCCCCCCC]

Embodiment 22 provides the LNP of any one of Embodiments 11-21, wherein the compound comprises about 1 mol % to about 99 mol % of the LNP.

Embodiment 23 provides the LNP of Embodiment 22, wherein the compound comprises about 50 mol % of the LNP.

Embodiment 24 provides the LNP of any one of Embodiments 11-23, wherein the at least one helper lipid comprises at least one selected from the group consisting of distearoylphosphatidylcholine (DSPC) and dioleoylphosphatidylethanolamine (DOPE).

Embodiment 25 provides the LNP of any one of Embodiments 11-24, wherein the at least one helper lipid comprises about 1 mol % to about 45 mol % of the LNP.

Embodiment 26 provides the LNP of Embodiment 25, wherein the at least one helper lipid comprises about 10 mol % of the LNP.

Embodiment 27 provides the LNP of any one of Embodiments 11-26, wherein the at least one cholesterol lipid comprises cholesterol.

Embodiment 28 provides the LNP of any one of Embodiments 11-27, wherein the at least one cholesterol lipid comprises about 5 mol % to about 50 mol % of the LNP.

Embodiment 29 provides the LNP of Embodiment 28, wherein the at least one cholesterol lipid comprises about 38.5 mol % of the LNP.

Embodiment 30 provides the LNP of any one of Embodiments 11-29, wherein the at least one conjugated lipid comprises C₁₄-PEG2000.

Embodiment 31 provides the LNP of any one of Embodiments 11-30, wherein the at least one conjugated lipid comprises about 0.1 mol % to about 12.5 mol % of the LNP.

Embodiment 32 provides the LNP of Embodiment 31, wherein the at least one conjugated lipid comprises about 1.5 mol % of the LNP.

Embodiment 33 provides the LNP of any one of Embodiments 11-32, wherein the ratio of (a):(b):(c):(d) is about 50:10:38.5:1.5.

Embodiment 34 provides the LNP of any one of Embodiments 11-33, wherein the LNP selectively binds to at least one sigma receptor.

Embodiment 35 provides the LNP of any one of Embodiments 11-34, wherein the LNP selectively binds to at least one target cell of interest.

Embodiment 36 provides the LNP of Embodiment 35, wherein the target cell of interest comprises cell expressing at least one sigma receptor.

Embodiment 37 provides the LNP of any one of Embodiments 11-36, wherein the LNP further comprises, or encapsulates, at least one additional agent.

Embodiment 38 provides the LNP of Embodiment 37, wherein the at least one additional agent is selected from the group consisting of a mRNA, a siRNA, a microRNA, a CRISPR-Cas9, a small molecule, a protein, an antibody, and any combination thereof.

Embodiment 39 provides the LNP of Embodiment 38, wherein the nucleic acid molecule is a DNA molecule or a RNA molecule.

Embodiment 40 provides the LNP of Embodiment 38 or 39, wherein the nucleic acid molecule is selected from the group consisting of cDNA, mRNA, miRNA, siRNA, modified RNA, antagomir, antisense molecule, targeted nucleic acid, and any combination thereof.

Embodiment 41 provides the LNP of any one of Embodiments 37-40, wherein the LNP further comprises mRNA.

Embodiment 42 provides the LNP of Embodiment 41, wherein the LNP has a ratio of lipids:mRNA of about 10:1.

Embodiment 43 provides a pharmaceutical composition comprising the LNP of any one of Embodiments 11-42 and at least one pharmaceutically acceptable carrier.

Embodiment 44 provides a method of delivering an agent to a subject in need thereof, the method comprising administering to the subject a therapeutically effective amount of at least one LNP of any one of Embodiments 11-42, wherein the agent is at least partially encapsulated in the LNP.

Embodiment 45 provides the method of Embodiment 44, wherein the LNP is administered as a pharmaceutical composition comprising at least one pharmaceutically acceptable carrier.

Embodiment 46 provides the method of Embodiment 44 or 45, wherein the agent is selected from the group consisting of a mRNA, a siRNA, a microRNA, a CRISPR-Cas9, a small molecule, a protein, an antibody, and any combination thereof.

Embodiment 47 provides the method of Embodiment 46, wherein the nucleic acid molecule is a DNA molecule or a RNA molecule.

Embodiment 48 provides the method of Embodiment 46 or 47, wherein the nucleic acid molecule is selected from the group consisting of cDNA, mRNA, miRNA, siRNA, modified RNA, antagomir, antisense molecule, targeted nucleic acid, and any combination thereof.

Embodiment 49 provides the method of any one of Embodiments 44-48, wherein the agent comprises mRNA.

Embodiment 50 provides the method of Embodiment 49, wherein the LNP has a ratio of lipids:mRNA of about 10:1.

Embodiment 51 provides the method of any one of Embodiments 44-50, wherein the LNP selectively binds at least one sigma receptor.

Embodiment 52 provides the method of any one of Embodiments 44-51, wherein the LNP selectively binds to at least one target cell of interest.

Embodiment 53 provides the method of Embodiment 52, wherein the target cell of interest comprises a cell expressing at least one sigma receptor.

Embodiment 54 provides a method of treating, preventing, and/or ameliorating a disease or disorder in a subject, the method comprising administering to the subject the LNP of any one of Embodiments 11-42 and/or the pharmaceutical composition of Embodiment 43.

Embodiment 55 provides the method of Embodiment 54, wherein the disease or disorder is fibrosis.

Embodiment 56 provides the method of Embodiment 54 or 55, wherein the disease or disorder is selected from the group consisting of a fibrotic disease or disorder, a disease or disorder associated with fibrosis, a neurological disease or disorder, a skin condition, a disease or disorder associated with the level of activity of at least one sigma receptor, a cancer or a disease or disorder associated therewith, and any combination thereof.

Embodiment 57 provides the method of Embodiment 56, wherein the fibrotic disease or disorder is at least one selected from the group consisting of pulmonary fibrosis, cystic fibrosis, fibrothorax, idiopathic pulmonary fibrosis, bridging fibrosis, cirrhosis, glial scar, liver fibrosis, myocardial fibrosis, interstitial fibrosis, replacement fibrosis, arterial stiffness, arthrofibrosis, chronic kidney disease, Crohn's disease, dupuytren's contracture, keloid, mediastinal fibrosis, myelofibrosis, peyronie's disease, nephrogenic systemic fibrosis, progressive massive fibrosis, retroperitoneal fibrosis, scleroderma, systemic sclerosis, adhesive capsulitis, and any combination thereof.

Embodiment 58 provides a modified cell produced by administering at least one LNP of any one of Embodiments 11-42 or a composition comprising the same to a cell.

Embodiment 59 provides the modified cell of Embodiment 58, wherein the cell expresses a chimeric antigen receptor (CAR).

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A compound of Formula (I), or a salt, solvate, stereoisomer, tautomer, or isotopologue thereof: 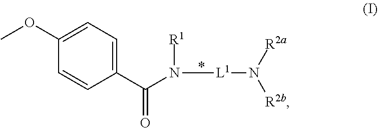
wherein:
L¹ is selected from the group consisting of -(optionally substituted C₂-C₁₂ heteroalkylenyl)- and -(optionally substituted C₂-C₁₂ heteroalkylenyl)-(optionally substituted C₂-C₁₂ heterocycloalkylenyl)-,
wherein each occurrence of C₂-C₁₂ heteroalkylenyl and C₂-C₁₂ heterocycloalkylenyl is optionally substituted with at least one substituent selected from the group consisting of optionally substituted C₁-C₂₄ alkyl, optionally substituted C₁-C₂₄ heteroalkyl optionally substituted C₃-C₈ cycloalkyl, optionally substituted C₆-C₁₀ aryl, optionally substituted C₂-C₁₀ heteroaryl, R^{2c}, and 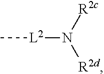
or two geminal substituents may combine to form ═O;
each occurrence of L² is independently selected from the group consisting of -(optionally substituted C₁-C₆ alkylenyl)-, -(optionally substituted C₂-C₆ heteroalkylenyl)-, -(optionally substituted C₁-C₆ alkylenyl)-C(═O)X-(optionally substituted C₁-C₆ alkylenyl)-, and -(optionally substituted C₁-C₆ alkylenyl)-XC(═O)-(optionally substituted C₁-C₆ alkylenyl)-;
R¹ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl;
each occurrence of R^{2a}, R^{2b}, R^{2c}, and R^{2d}, if present, is independently selected from the group consisting of H, -(optionally substituted C₁-C₆ alkylenyl)-C(═O)OR³, -(optionally substituted C₁-C₆ alkylenyl)-C(═O)N(R³)(R⁴), -(optionally substituted C₁-C₆ alkylenyl)-C(═O)R³, -(optionally substituted C₁-C₆ alkylenyl)-(R³), —C(═O)OR³, —C(═O)N(R³)(R⁴), —C(═O)R³, and R³,
wherein no more than one of each occurrence of R^{2a}, R^{2b}, R^{2c}, and R^{2d} is H;
R³ is selected from the group consisting of optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
R⁴ is selected from the group consisting of H and optionally substituted C₁-C₆ alkyl;
each occurrence of X is independently selected from the group consisting of a bond, O, and NR⁵; and
each occurrence of R⁵ is selected from the group consisting of H, optionally substituted C₁-C₆ alkyl, optionally substituted C₃-C₈ cycloalkyl, optionally substituted C₂-C₈ heterocycloalkyl, optionally substituted C₆-C₁₀ aryl, and optionally substituted C₂-C₁₀ heteroaryl.

2. The compound of claim 1, wherein R¹ is H.

3. The compound of claim 1, wherein L¹ is selected from the group consisting of: 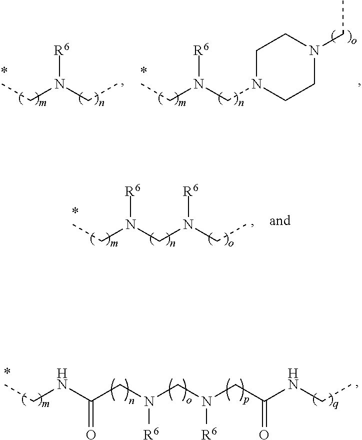
wherein:
each occurrence of R⁶ is independently selected from the group consisting of H, optionally substituted C₁-C₂₄ alkyl, and 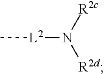
m, n, o, p, and q are each independently 1, 2, 3, or 4.

4. The compound of claim 3, wherein R⁶ is selected from the group consisting of H, CH₃, CH₂CH(OH)(optionally substituted C₁-C₁₂ alkyl) and

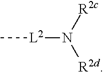

5. The compound of claim 1, wherein L² is selected from the group consisting of —CH₂CH₂—, —CH₂CH₂CH₂—, and —CH₂CH₂—C(═O)NH—CH₂CH₂—.

6. The compound of claim 1, wherein L¹ is selected from the group consisting of:

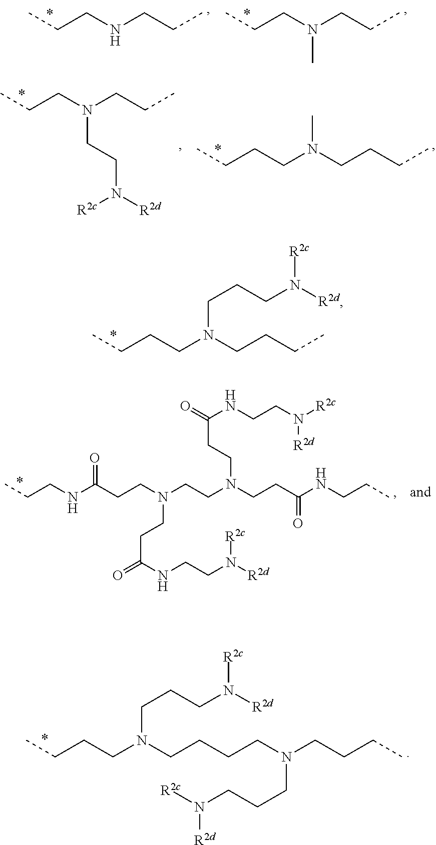

7. The compound of claim 1, wherein R^{2a}, R^{2b}, R^{2c}, and R^{2d}, if present, are each independently selected from the group consisting of H, —CH₂CH(OH)(optionally substituted C₁-C₂₈ alkyl), —CH₂CH(OH)(optionally substituted C₂-C₂₈ alkenyl), —CH₂CH₂C(═O)O(optionally substituted C₁-C₂₈ alkyl), and —CH₂CH₂C(═O)NH(optionally substituted C₁-C₂₈ alkyl),
optionally wherein R^{2a}, R^{2b}, R^{2c}, and R^{2d}, if present, are each independently selected from the group consisting of —CH₂CH(OH)(CH₂)₉CH₃, —CH₂CH(OH)CH₂)₁₁CH₃, and —CH₂CH(OH)CH₂)₁₃CH₃.

8. (canceled)

9. The compound of claim 1, wherein each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, and optionally substituted heteroalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R¹, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

10. The compound of claim 1, which is selected from the group consisting of:

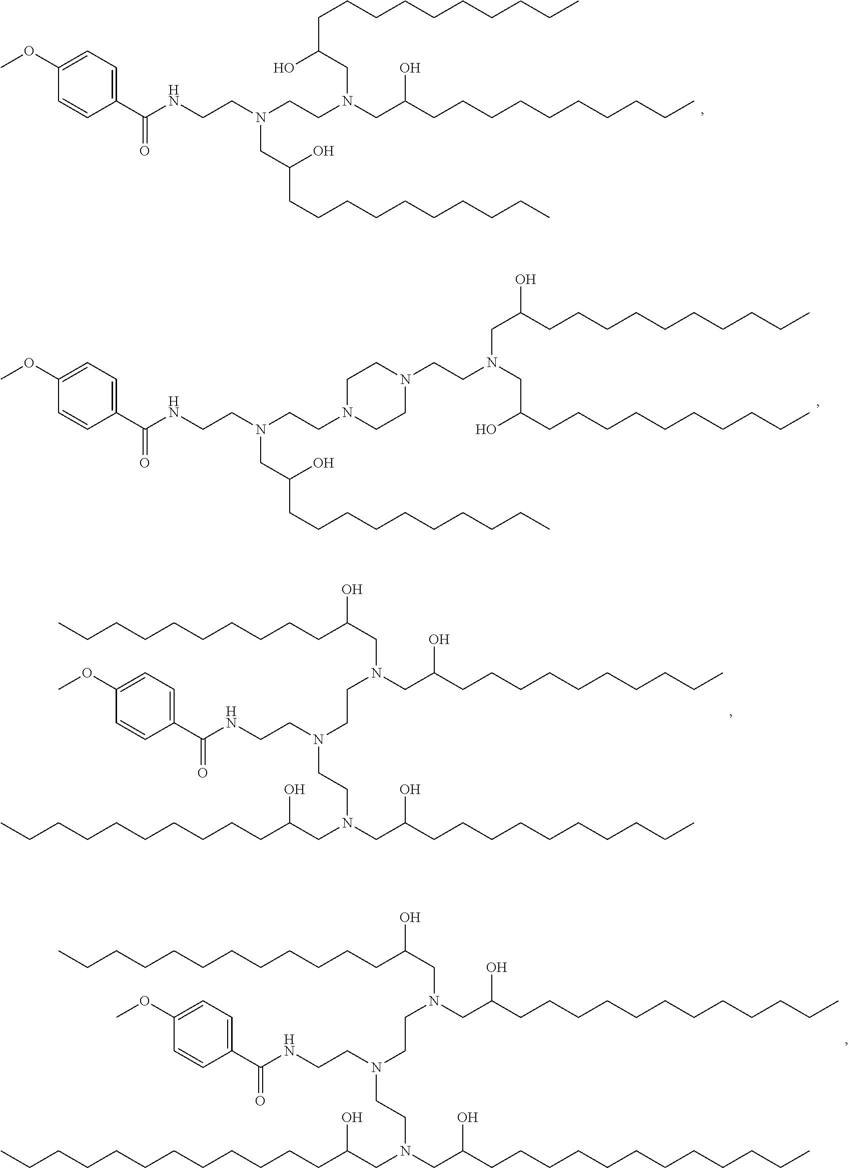

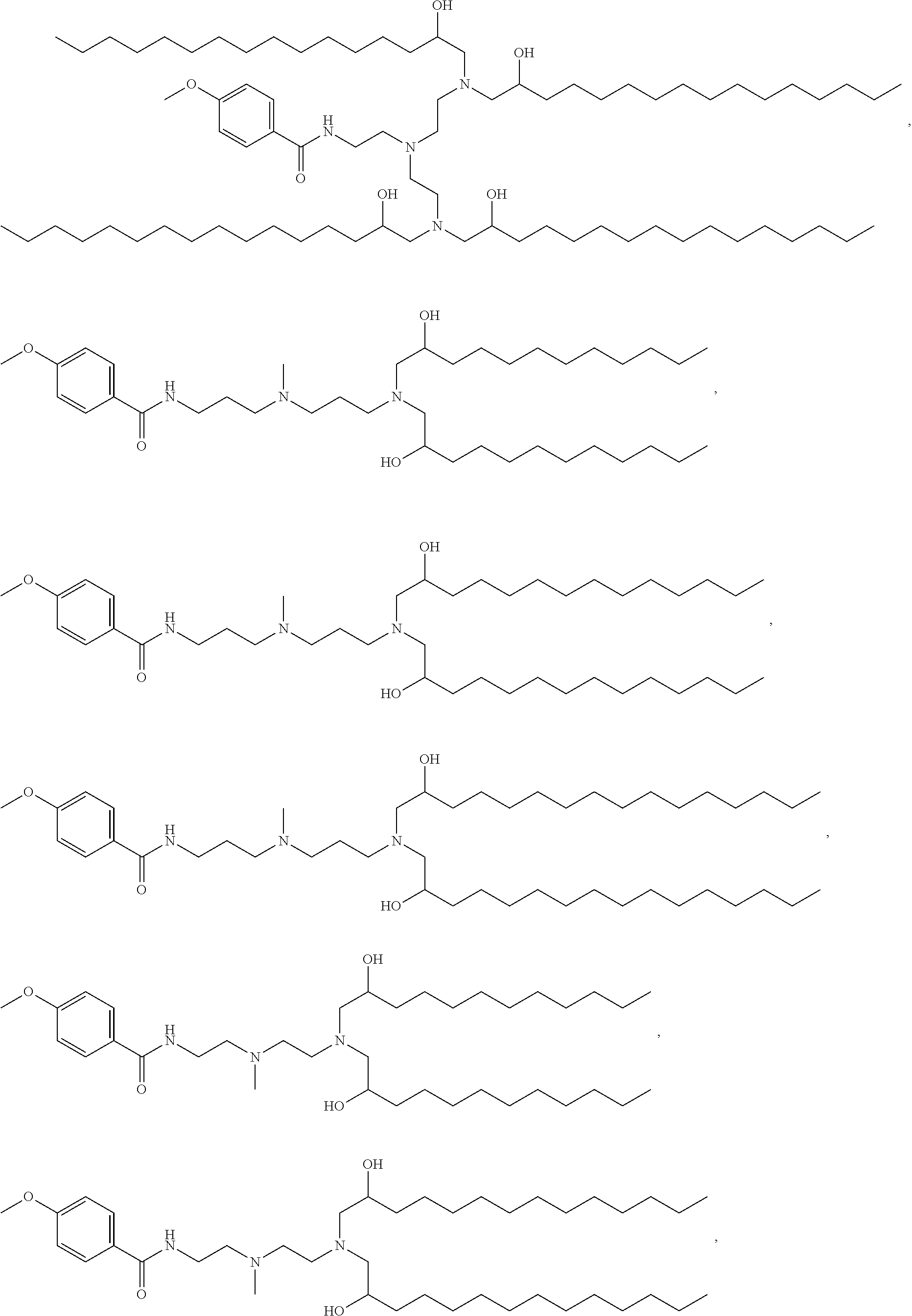

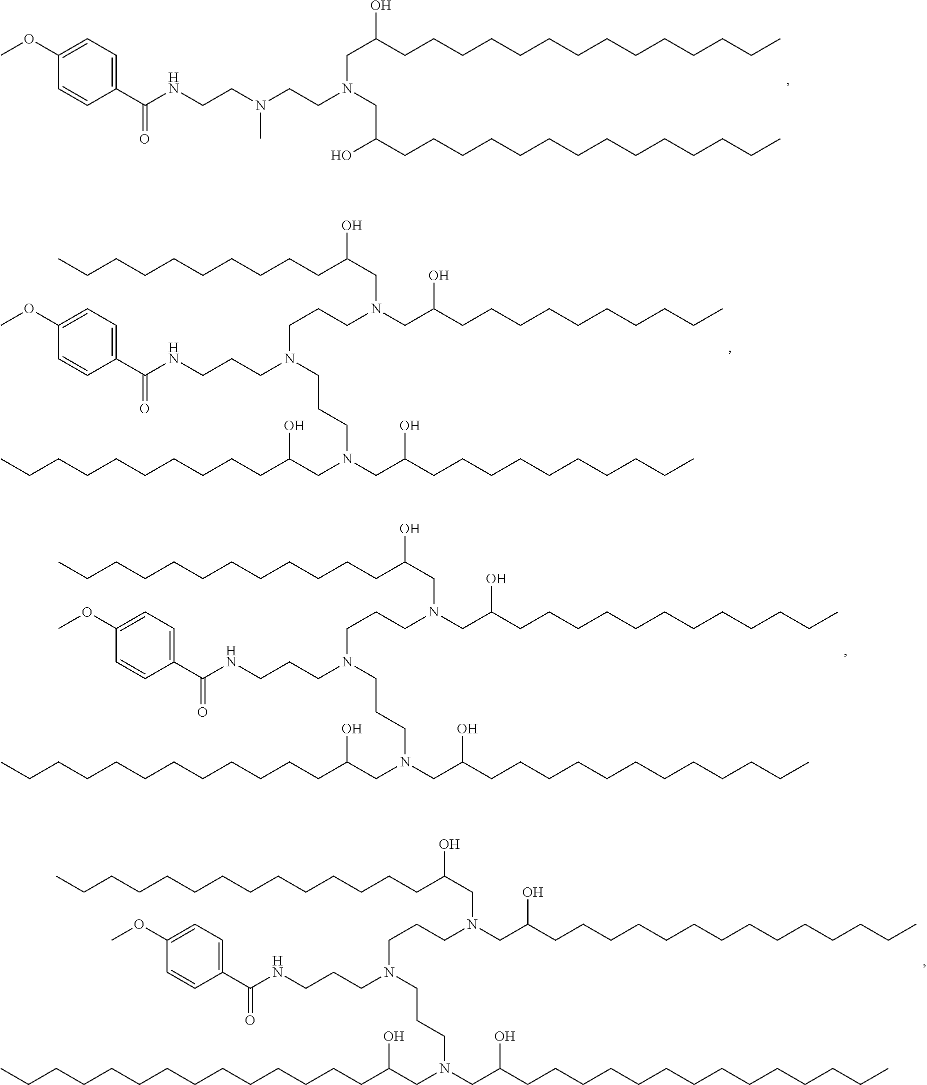

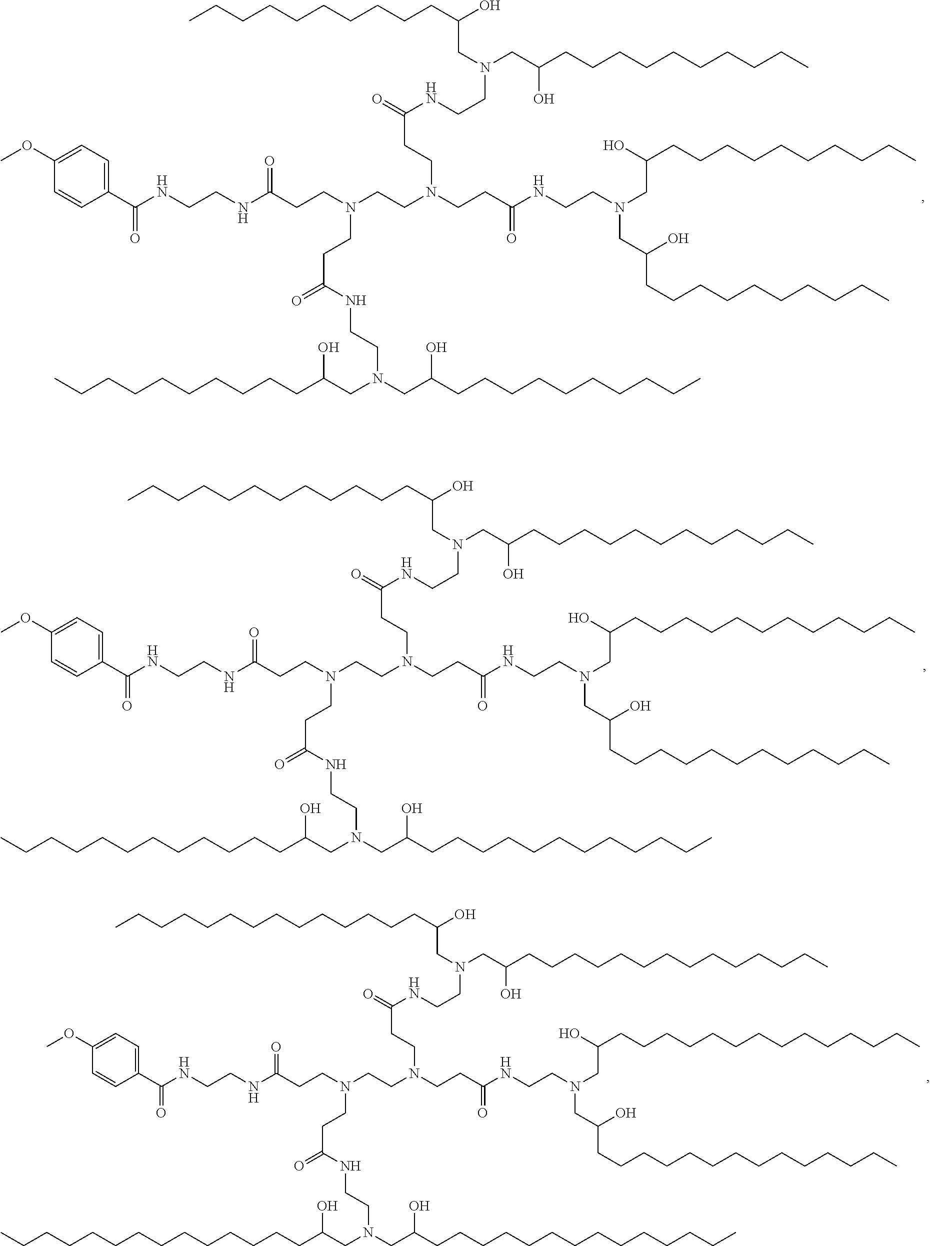

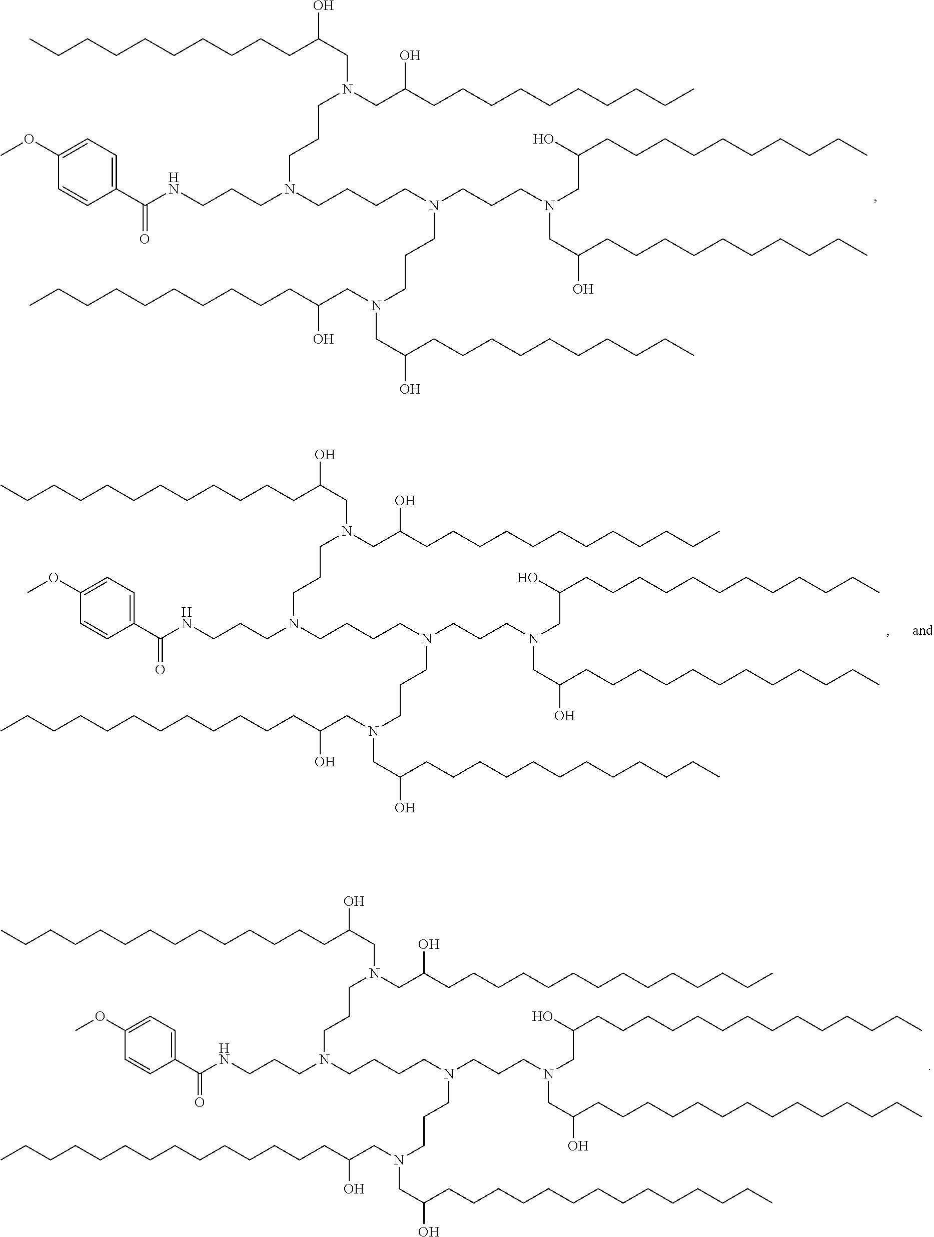

11. A lipid nanoparticle (LNP) comprising:
(a) at least one compound of claim 1;
(b) at least one helper lipid;
(c) at least one cholesterol lipid; and
(d) at least one conjugated lipid.

12. The LNP of claim 11, wherein at least one of the following applies:
(a) R¹ is H;
(b) L¹ is selected from the group consisting of 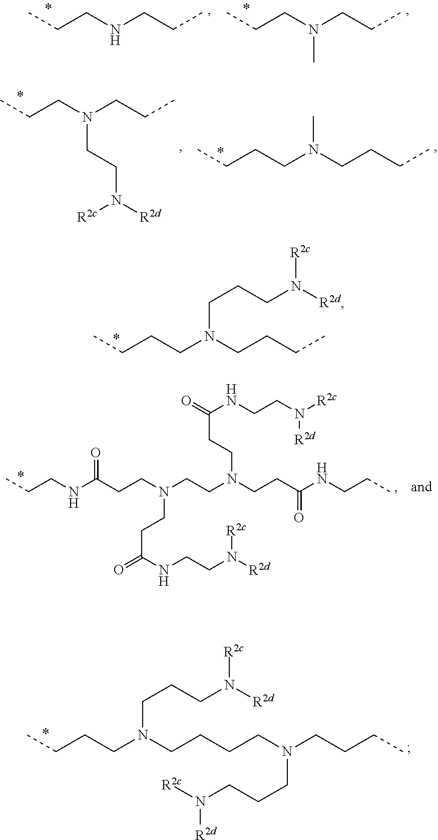
(c) L² is selected from the group consisting of —CH₂CH₂—, —CH₂CH₂CH₂—, and —CH₂CH₂—C(═O)NH—CH₂CH₂—;
(c) R^{2a}, R^{2b}, R^{2c}, and R^{2d}, if present, are each independently selected from the group consisting of H, —CH₂CH(OH)(optionally substituted C₁-C₂₈ alkyl), —CH₂CH(OH)(optionally substituted C₂-C₂₈ alkenyl), —CH₂CH₂C(═O)O(optionally substituted C₁-C₂₈ alkyl), and —CH₂CH₂C(═O)NH(optionally substituted C₁-C₂₈ alkyl), optionally wherein R^{2a}, R^{2b}, R^{2c}, and R^{2d}, if present, are each independently selected from the group consisting of —CH₂CH(OH)(CH₂)₉CH₃, —CH₂CH(OH)CH₂)₁₁CH₃, and —CH₂CH(OH)(CH₂)₁₃CH₃; and
(d) each occurrence of optionally substituted alkyl, optionally substituted heteroalkyl, optionally substituted cycloalkyl, optionally substituted heterocycloalkyl, optionally substituted alkenyl, optionally substituted alkynyl, optionally substituted aryl, optionally substituted heteroaryl, optionally substituted aralkyl, optionally substituted alkylenyl, and optionally substituted heteroalkylenyl, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

13-19. (canceled)

20. The LNP of claim 1, which is selected from the group consisting of:

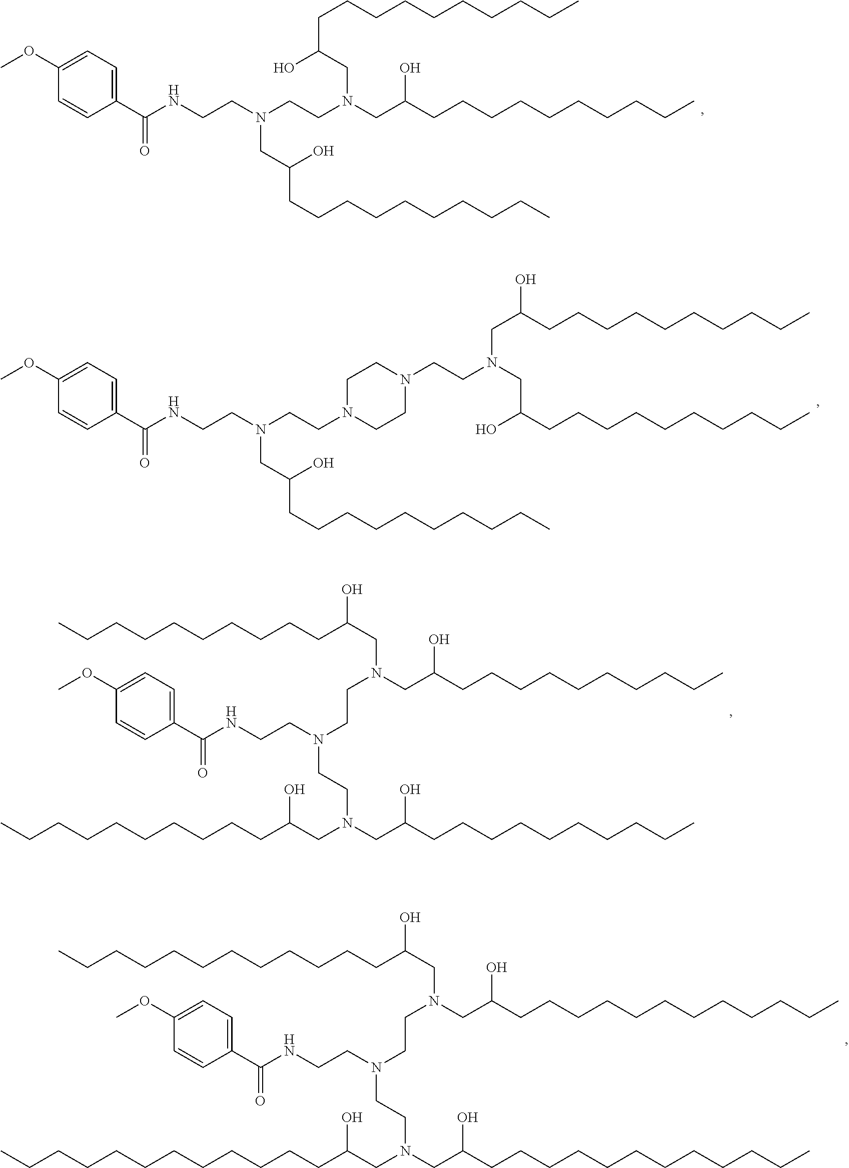

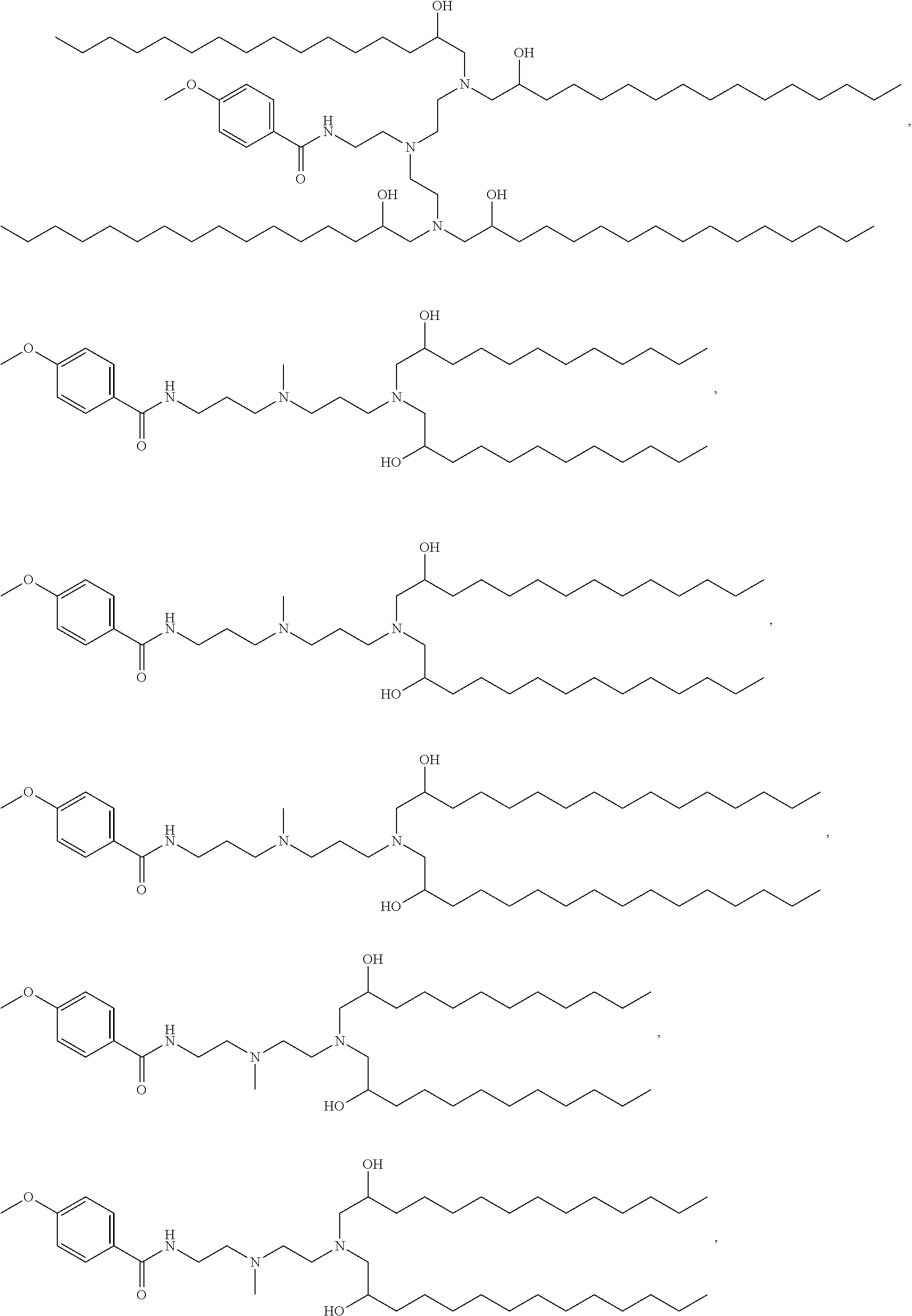

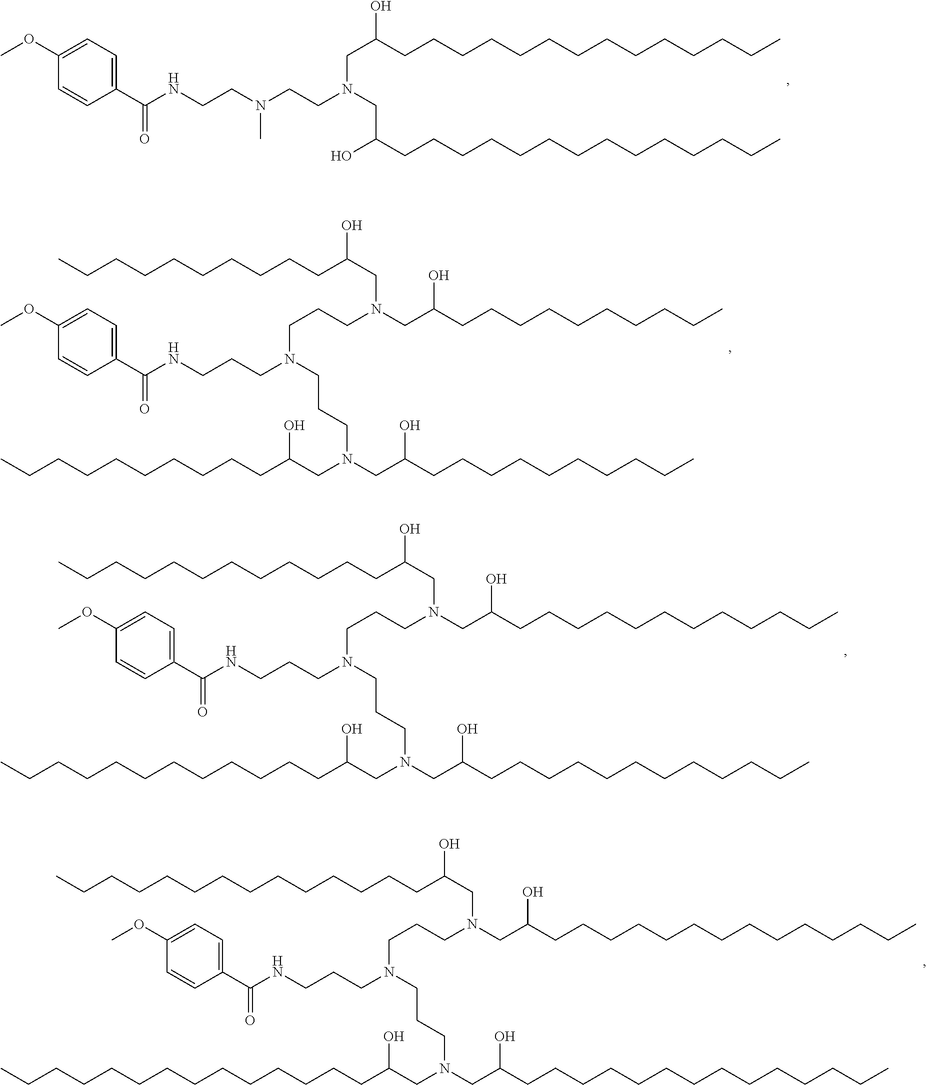

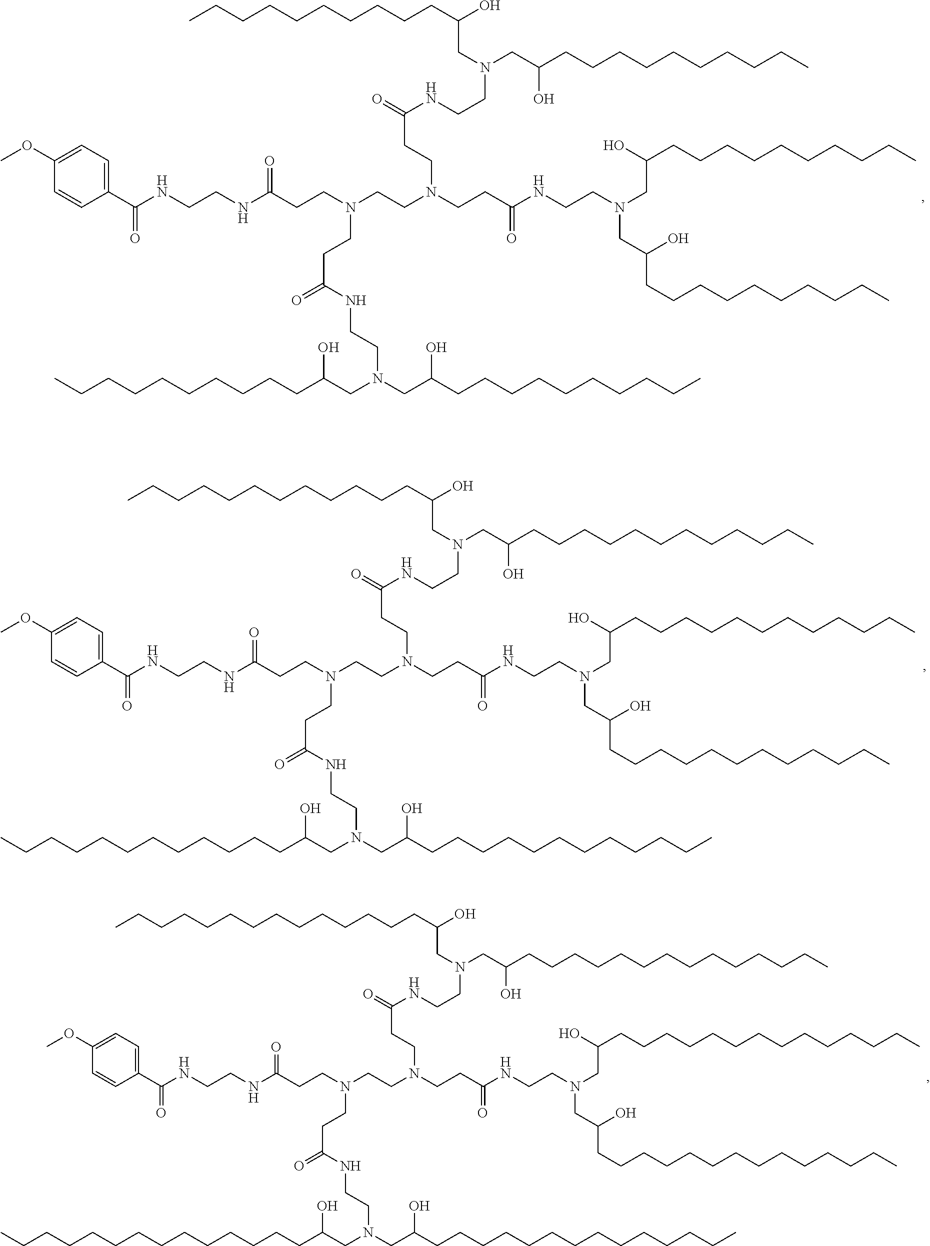

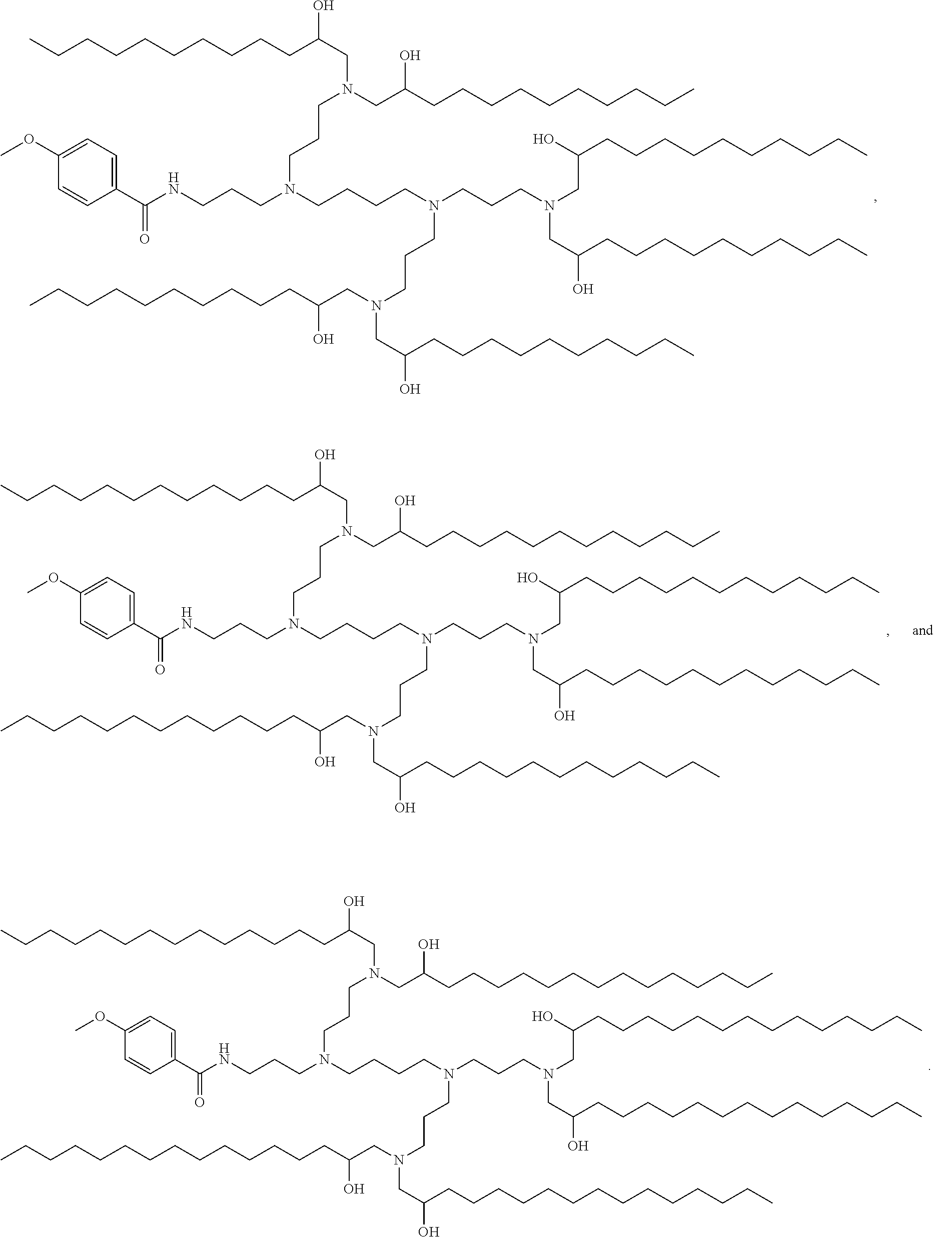

21. (canceled)

22. The LNP of claim 11, wherein:
(a) the compound comprises about 1 mol % to about 99 mol % of the LNP, optionally wherein the compound comprises about 50 mol % of the LNP;
(b) the at least one helper lipid comprises about 1 mol % to about 45 mol % of the LNP, optionally wherein the at least one helper lipid comprises about 10 mol % of the LNP;
(c) the at least one helper lipid comprises at least one selected from the r NR consisting of distearoylphosphatidylcholine (DSPC) and dioleoylphosphatidylethanolamine (DOPE);
(d) the at least one cholesterol lipid comprises about 5 mol % to about 50 mol % of the LNP, optionally wherein the at least one cholesterol lipid comprises about 38.5 mol % of the LNP;
(e) the at least one cholesterol lipid comprises cholesterol;
(f) the at least one conjugated lipid comprises about 0.1 mol % to about 12.5 mol % of the LNP, optionally wherein the at least one conjugated lipid comprises about 1.5 mol % of the LNP;
(g) the at least one conjugated lipid comprises C14-PEG2000; and
(d) the ratio of (a):(b):(c):(d) is about 50:10:38.5:1.5.

23-33. (canceled)

34. The LNP of claim 11, wherein at least one of the following applies:
(a) the LNP selectively binds to at least one sigma receptor;
(b) the LNP selectively binds to at least one target cell of interest, optionally wherein the target cell of interest comprises cell expressing at least one sigma receptor;
(c) the LNP further comprises, or encapsulates, at least one additional agent, optionally wherein the at least one additional agent is selected from the group consisting of a DNA molecule, RNA molecule, a CRISPR-Cas9, a small molecule, a protein, an antibody, and any combination thereof, optionally wherein the DNA or RNA is selected from the group consisting of cDNA, mRNA, miRNA, siRNA, modified RNA, antagomir, antisense molecule, and a targeted nucleic acid, or any combination thereof, and optionally wherein the LNP has a ratio of lipids:mRNA of about 10:1.

35-42. (canceled)

43. A pharmaceutical composition comprising the LNP of claim 11 and at least one pharmaceutically acceptable carrier.

44. A method of delivering an agent to a subject in need thereof, the method comprising administering to the subject a therapeutically effective amount of at least one LNP of claim 11, wherein the agent is at least partially encapsulated in the LNP.

45. The method of claim 44, wherein at least one of the following applies:
(a) the LNP is administered as a pharmaceutical composition comprising at least one pharmaceutically acceptable carrier;
(b) the agent is selected from the group consisting of a DNA molecule, RNA molecule, a CRISPR-Cas9, a small molecule, a protein, an antibody, and any combination thereof, optionally wherein the DNA or RNA is selected from the group consisting of cDNA, mRNA, miRNA, siRNA, modified RNA, antagomir, antisense molecule, and a targeted nucleic acid, or any combination thereof, and optionally wherein the LNP has a ratio of lipids:mRNA of about 10:1;
(c) the LNP selectively binds at least one sigma receptor; and
(d) the LNP selectively binds to at least one target cell of interest, optionally wherein the target cell of interest comprises a cell expressing at least one sigma receptor.

46-53. (canceled)

54. A method of treating, preventing, and/or ameliorating a disease or disorder in a subject, the method comprising administering to the subject the LNP of claim 11 and/or a pharmaceutical composition thereof.

55. (canceled)

56. The method of claim 54, wherein the disease or disorder is selected from the group consisting of a fibrotic disease or disorder, a disease or disorder associated with fibrosis, a neurological disease or disorder, a skin condition, a disease or disorder associated with the level of activity of at least one sigma receptor, a cancer or a disease or disorder associated therewith, and any combination thereof,
optionally wherein the fibrotic disease or disorder is at least one selected from the group consisting of pulmonary fibrosis, cystic fibrosis, fibrothorax, idiopathic pulmonary fibrosis, bridging fibrosis, cirrhosis, glial scar, liver fibrosis, myocardial fibrosis, interstitial fibrosis, replacement fibrosis, arterial stiffness, arthrofibrosis, chronic kidney disease, Crohn's disease, dupuytren's contracture, keloid, mediastinal fibrosis, myelofibrosis, peyronie's disease, nephrogenic systemic fibrosis, progressive massive fibrosis, retroperitoneal fibrosis, scleroderma, systemic sclerosis, adhesive capsulitis, and any combination thereof.

57. (canceled)

58. A modified cell produced by administering at least one LNP of claim 11 or a composition comprising the same to a cell,
optionally wherein the cell expresses a chimeric antigen receptor (CAR).

59. (canceled)
